SEQUENCE LISTING (30 sequences)

SEQ ID NO: 1 - protein, 17 aa, synthetic construct
  source 1..17: note = insulin B9-25 epitope
SHLVEALYLVCGERGFF

SEQ ID NO: 2 - protein, 15 aa, synthetic construct
  source 1..15: note = T cell (Beta2GPI) epitope
KVSFFCKNKEKKCSY

SEQ ID NO: 3 - protein, 11 aa, synthetic construct
  source 1..11: note = B cell epitope
VSRGGMRKFIC

SEQ ID NO: 4 - DNA, 282 bp, synthetic construct
  source 1..282: mol_type = other DNA; note = primase ori from the PICI of the Escherichia coli strain CFT073
tttgttgcaatggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagccgcgccattactggcatggtggccagtaaggtagataaggtagacaaggggaggcacaactcaaaactttttaaacgagggggtaaaa

SEQ ID NO: 5 - skipped

SEQ ID NO: 6 - DNA, 282 bp, synthetic construct
  source 1..282: mol_type = other DNA; note = Primase ori deltaGAAABCC
tttgttgcaatggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagcagcgccattactggcatggtggccagtaaggtagataaggtagacaaggggaggcacaactcaaaactttttaaacgagggggtaaaa

SEQ ID NO: 7 - DNA, 282 bp, synthetic construct
  source 1..282: mol_type = other DNA; note = Primase ori devoid of restriction sites
tttgttgcaatggctgtctaccctgtctacctgagtaaagaaaaatacatttaattcagtatattaacttgggtagacagcctttttttactgtctaccttctgtctaccctctctacctgattttacctgaatcagacagggaggtagacacggggtagacagtggataaaagcactctaccccactgaaagcagtgccattactggcatggttgccagtaaggttgataaggtagacaaggggagggacaactcaaaactttttaaacgagggggtaaaa

SEQ ID NO: 8 - protein, 584 aa, synthetic construct
  source 1..584: note = PICI primase-helicase
MKLAPNVKQQSRGIKHKETEVIIFAGSDAWSHAKQWQEHDARMAGDNEPPVWLGEQQLSELDKLQIVPEGRKSVRIFRAGYLAPVMIKAIGQKLAAAGVQDANFYPDGMHGQKVENWREYLARERQNLSDGLVIELPVKQKAQLSQMADSERAQLLADRFDGVCVHPESEIVHVWCGGVWCPVSTMELSREMVAIYSEHRATFSKRVINNAVEALKVIAEPMGEPSGDLLPFANGALDLKTGEFSPHTPENWITTHNGIEYTPPAPGENIRDNAPNFHKWLEHAAGKDPRKMMRICAALYMIMANRYDWQMFIEATGDGGSGKSTFTHIASLLAGKQNTVSAEMTSLDDAGGRAQVVGSRLIVLADQPKYTGEGTGIKKITGGDPVEINPKYEKRFTAVIRAVVLATNNNPMIFTERAGGVARRRVIFRFDNIVSEAEKDRELPEKIAAEIPVIIRRLLANFADPEKARALLIEQRDGDEALAIKQQTDPVIEFCQFLNFLEEARGLMMGGGGDSVKYTTRNSLYRVYLAFMAYAGRSKPLNVNDFGKAMKPAAKVYGHEYITRKVKGVTQTNAITTDDCDAFL

SEQ ID NO: 9 - DNA, 1752 bp, synthetic construct
  source 1..1752: mol_type = other DNA; note = PICI primase-helicase
atgaaactggcaccgaacgtaaaacagcagtcacgcggcataaaacacaaagaaacagaagtcattatttttgcgggtagtgatgcctggtcacacgcaaaacaatggcaggaacatgacgcgcgtatggccggagataatgagcctcctgtgtggcttggggagcagcagttatccgaactggataagctgcaaattgtgccggaaggcagaaaatccgtgcgcatattcagggccggatatcttgcgccagtaatgataaaggcgattggtcagaagctggcggcggcaggcgtacaggatgcaaatttttaccctgatggtatgcacggtcagaaggtggagaactggcgcgaatatctggcccgtgagcgccagaatctttctgatggtctggtcattgagcttccggtaaagcaaaaggcgcaactttcgcagatggcggacagtgagcgcgcgcagctgcttgccgatcgctttgatggcgtttgcgtacatcctgaaagtgaaatcgttcacgtatggtgcggcggggtatggtgtccggtcagcacaatggagctgagccgcgaaatggtggcgatctattcagagcacagggccactttcagcaagcgcgtaatcaataacgccgtggaagcgttaaaagttattgccgaaccaatgggcgagccgtccggcgatttgctgccgttcgccaatggtgcgcttgacctgaaaacgggggaattttccccgcacacgccggagaactggatcaccacgcacaacggcattgagtacacgccaccagcacccggggagaacatccgcgataacgcgccaaactttcataaatggcttgagcacgcagccggaaaagacccgcgcaagatgatgcgtatatgtgccgcgctgtacatgattatggcgaaccggtacgactggcagatgtttattgaggccaccggagacggcgggagcggtaaaagtacattcacacacatagccagccttctggcagggaaacaaaacacggtaagcgctgaaatgacatcgcttgatgatgctggtgggcgtgcgcaggttgtcgggagtcgtcttatcgtcctggcagaccagccgaaatatacaggcgaaggaacgggcatcaagaaaatcacgggcggcgaccccgtggaaattaacccgaaatatgaaaagcgttttacggcggtaatcagggcggtggtgctggcaaccaataacaatccgatgatattcaccgaacgggccggaggtgtggcacgtcgtcgggtgatattccggttcgataacatcgtaagcgaggcagaaaaagacagggagctaccggaaaagatcgcggctgaaatccctgtcattatccgccgcttgctggcgaactttgccgaccctgaaaaggcacgggctttactcattgaacagcgtgacggtgatgaagcactggcaataaagcaacagacggatccggttattgagttttgccagttcctgaattttctggaggaagcacgcggcctgatgatgggcggcggtggcgattcagtgaagtacacgaccagaaacagcctttaccgcgtctatctggcgtttatggcgtacgcaggcaggagcaaaccgctaaacgtaaatgactttggcaaggctatgaagccagccgcgaaagtttacggacatgaatatattacgcggaaagttaaaggagtaacgcagactaacgcaataacaacagacgattgcgacgcgttttta

SEQ ID NO: 10 - DNA, 11615 bp, synthetic construct
  source 1..11615: mol_type = other DNA; note = payload p1392 plasmid
gtttgcaataagggacaagttacgagtgtagacacgcagaattatccagcctttagtctttaggaaggcaaagctattgtacgcggtagccgtcgtagcaatttaccaactgtagaattattggacacacgtaacaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgatcggaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttccacggtcaagcacaatcagctagcaagatcttggaatagagtcgttgcaccgctttgatttacatgctctccattgcacaacattccggaaggactggcttctctgccatgatcggataatgaaaaacatcagtatgccctgtcatttttctttgggtgtcctcaaataattgccctcacgttatcgtatgtgacgcgctcatctatgctcgaagtattccttgttctcccatcttttaatagaaagtctttaatgaacgtgtcgttacgcagtgtatgaactcttgttttatagggcagactttggcgtggcctaagtgtgttcgataagaaggcaaggacaactagctgacgcgctgtaatacggatattatggcacggttgatacaaacgctgatatcctgatttgctaatgtgcccaacactttagttgagtgccacgttccgactacaagttgcttcaagaggggaatttggatttggcaatagccccccgtttctacctcaagaggcgacgagtattaaccgcgccagctttcggcacaagggccaaagaagattccaatttcttattcccgaataacctccgaatccctgcgggaaaatcaccgaccgaatagcctagaagcaagggggaacagataggtataattagcttaagagagtaccagccgtgacaacaccgtagtaaccacaaacttacgctggggcttctttggcggatttttacagatactaacaaggtgatttgaagtaccttagttgaggatttaaacgcgctatccggtagtctacaaattgggaaataccgttcaaagagggctagaattacttaaaagccttcacaccgcctgcgctatacgcgcccactctcccgtttatccgtccaagcggaagcagggcgaacttccgctaagatattcttacgtgtaacgtagctaagtatcccaaatagctggcgtacgcgttgaacaccgcctagaggatcgggagtcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagttctaatctcttgcccccgcccgtaatagcctccaagtgattcaagatagtaaagggcaagagcttattcggcgttgaaggatagcggactttcggtcaaccacaattccccactcgacaaaaccagccgtgcgaagaactctgaaagtacaagcaacccaagagggctgagcctaaactcagctaattcctaagtgagctaaagactcgaagtgacagctattaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtggatagccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccagaaatcggcatggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagcttagagcttgcgtagcacttagccacaagttaattaacagttgtctggtagtttggcggtattagcgagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagggtaagtagttcaccctatagtacgaagcagaactatttcgaggggcgtgcaataatcgaatcttctgcggttgacttaacacgctagggacgtgccctcgattcagtcgcaggtactcctactcagactgcctcacacccagctagtcactgagcgataaaattgacccgccctctaaggtagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgagggcgattgtttctgcgaaaggtgttgtcctaatagtcgcgacatttggcccttgtaggtgtgaaaccacttagcttcgcgccgtagtcctaaaggcccacctattgactttgtttcgggtagcactaggaatcttaacaatttgaatttggacgtggaacgcgtacaccttgatcttcgaataattctagggatttggaagtcctctacgttgacacacctacaatgctccaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtccggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcactaccaaagaaaaataaaaagacgctgaaaagcgtctttttatttttcggtccagtgtaactcaggcaaaagcacgtaatattcgtactcaccaaacgaaactcatccggcgcatcgcgcttcttcctccgtaagcgtcacccccattacttaaagagtgcatgtgcatattttgttatcaataaaaaaggccgcgatttgcggccttattgttcgtcttgccggattagatagctaccggtgctttaatacccggatgcggatcatagccttcgatttcgaagtcctcaaaacgataatcgaagatgctttccggtttgcgtttgataatcagtttcgggagcgggcgtggctcacggcttaattgtaaatgcgtctgatccatgtgatttgagtacaggtgagtatccccaccagtccaaacaaagtcaccaacttccagatcacactgctgtgccatcatatgaactaataaggcgtaggaggcaatgttaaacggtaagcccagaaacacgtcgcaagaacgctggtacagttggcacgataacttaccatccgcaacatagaattgaaagaaggcatgacacggtgctaaagccattttgtctaattcccccacgttccatgcggacacgataatccggcgagagtccggatcatttttcagttggttaagaacggtagtgatctgatcaatatgccgaccatccggcgtaggccatgcacgccattgcttaccatacactggccctaagtcaccgttttcatctgcccactcatcccagatggtaacgttattctcgtgcaggtacgcaatgttcgtatcgccttgcagaaaccataataactcgtgaataatagaacggaggtggcaacgcttggtagtgaccagcgggaaaccgtcttgcaggttgaaacgcatctgatgaccaaagatagacagcgtaccagtgccagtacgatcattcttctgagtgccttcgtccagcactttttgcatcagttccagatactgtttcattttagcttccttagcttgcgaaatctcgataactcaaaaaatagtagtgatcttatttcattatggtgaaagttgtcttacgtgcaacattttcgcaaaaagttggcgctttatcaacactgtccgaatgacaaatggttacaattattgaacacccttcggggtgtttttttgtttctggtttcccgaggccgaacttttgttgcaatggctgtctaccctgtctacctgagtaaagaaaaatacatttaattcagtatattaacttgggtagacagcctttttttactgtctaccttctgtctaccctctctacctgattttacctgaatcagacagggaggtagacacggggtagacagtggataaaagcactctaccccactgaaagcagtgccattactggcatggttgccagtaaggttgataaggtagacaaggggagggacaactcaaaactttttaaacgagggggtaaaacgcagatcaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtttcccgaatagagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaagaacctgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcctcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctctatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtcgaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtcaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtacctctctacaataaaatccgcaactacatcacgcagaagccttacagtgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaacaaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaaattcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgcccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaaagtgtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagacggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagcttaccgctccgttcgaaacgtttaagaaaatgggtaaacaaacggggattatctattatgtgccagccggtttcacctccaagatttgtccagttacgggcttcgttaaccagctttacccgaaatacgagagcgttagcaaatctcaagaatttttcagcaaattcgacaagatctgctataatctggataaaggctatttcgagttcagctttgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctccaaatgcgtaattctaaaacgggtacggagcttgactacctgatttctccggtagccgacgttaacggcaacttcttcgattctcgtcaagcaccgaaaaatatgccacaagacgcggatgccaacggtgcataccatatcggccttaaaggcttaatgttattaggccgtatcaagaataatcaggagggcaagaaattaaatctggttatcaaaaacgaagaatacttcgagttcgttcagaatcgtaacaattaatgtatgcttaagcagatcggtaataaagacgaacaataagacgctgaaaagcgtcttttttcgttttggtcctgttccggcgcgatagtgtgaacatgctatagacttctggtgctacccgactgacaattaatcatccggctcgtataatgctagcaatttctactgttgtagatcattccggaacgttccagcgctgcaatttctactgttgtagatctgatttttcacatgttacctttcaatttctactgttgtagatccgaaaacgtaaagcttcagctgtaatttctactgttgtagatatcatatctggcgttaatggagtttcgtgacgaacaataagtcctccctaacggggggcaatttttattgataacaaaagtaacttcgagcttgtctacctcctagctcgtaaattgcacgctgatagtctcccaattgcgaaggaccaaaacgaaaaaacaccctttcgggtgtcttttctggaatttggtacgcagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaatactaagttgggataactccgtaactgactacgcctttctctagactttacttgaccagatacactgtctttgacacgttgaaggattagagcaatcaaatccaagactggctaagcacgaagcaactcttgagtgttaaaaagttacttcctgtattcgggacgagggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaagtgaagccgacacctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcg

SEQ ID NO: 11 - DNA, 11609 bp, synthetic construct
  source 1..11609: mol_type = other DNA; note = payload p1900 plasmid
tcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagggtaagtagttcaccctatagtacgaagcagaactatttcgaggggcgtgcaataatcgaatcttctgcggttgacttaacacgctagggacgtgccctcgattcagtcgcaggtactcctactcagactgcctcacacccagctagtcactgagcgataaaattgacccgccctctaaggtagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgagggcgattgtttctgcgaaaggtgttgtcctaatagtcgcgacatttggcccttgtaggtgtgaaaccacttagcttcgcgccgtagtcctaaaggcccacctattgactttgtttcgggtagcactaggaatcttaacaatttgaatttggacgtggaacgcgtacaccttgatcttcgaataattctagggatttggaagtcctctacgttgacacacctacaatgctccaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtccggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcactaccaaagaaaaataaaaagacgctgaaaagcgtctttttatttttcggtccagtgtaactcaggcaaaagcacgtaatattcgtactcaccaaacgaaactcatccggcgcatcgcgcttcttcctccgtaagcgtcacccccattacttaaagagtgcatgtgcatattttgttatcaataaaaaaggccgcgatttgcggccttattgttcgtcttgccggattagatagctaccggtgctttaatacccggatgcggatcatagccttcgatttcgaagtcctcaaaacgataatcgaagatgctttccggtttgcgtttgataatcagtttcgggagcgggcgtggctcacggcttaattgtaaatgcgtctgatccatgtgatttgagtacaggtgagtatccccaccagtccaaacaaagtcaccaacttccagatcacactgctgtgccatcatatgaactaataaggcgtaggaggcaatgttaaacggtaagcccagaaacacgtcgcaagaacgctggtacagttggcacgataacttaccatccgcaacatagaattgaaagaaggcatgacacggtgctaaagccattttgtctaattcccccacgttccatgcggacacgataatccggcgagagtccggatcatttttcagttggttaagaacggtagtgatctgatcaatatgccgaccatccggcgtaggccatgcacgccattgcttaccatacactggccctaagtcaccgttttcatctgcccactcatcccagatggtaacgttattctcgtgcaggtacgcaatgttcgtatcgccttgcagaaaccataataactcgtgaataatagaacggaggtggcaacgcttggtagtgaccagcgggaaaccgtcttgcaggttgaaacgcatctgatgaccaaagatagacagcgtaccagtgccagtacgatcattcttctgagtgccttcgtccagcactttttgcatcagttccagatactgtttcattttagcttccttagcttgcgaaatctcgataactcaaaaaatagtagtgatcttatttcattatggtgaaagttgtcttacgtgcaacattttcgcaaaaagttggcgctttatcaacactgtccgaatgacaaatggttacaattattgaacacccttcggggtgtttttttgtttctggtttcccgaggccgaacttttgttgcaatggctgtctaccctgtctacctgagtaaagaaaaatacatttaattcagtatattaacttgggtagacagcctttttttactgtctaccttctgtctaccctctctacctgattttacctgaatcagacagggaggtagacacggggtagacagtggataaaagcactctaccccactgaaagcagtgccattactggcatggttgccagtaaggttgataaggtagacaaggggagggacaactcaaaactttttaaacgagggggtaaaacgcagatcaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtttcccgaatagagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaagaacctgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgaccaaaacgtttgatagcgagttttttaacctgtacagcctgcaaaaaaccgtgcgctttgaattaaaaccagtgggcgaaaccgcgagctttgtggaagattttaaaaacgaaggcctgaaacgtgtggttagcgaagatgaacgccgtgcggtggattatcagaaagtgaaagaaattattgatgattatcatcgcgattttattgaagaaagtctgaactattttccggaacaggtgagcaaagatgcgctggaacaggcgtttcatctgtatcagaaattaaaggccgcgaaagttgaagaaagagaaaaagcgctgaaagaatgggaagcactgcaaaaaaaactgcgtgaaaaagtggtgaaatgctttagcgatagcaataaagcgcgtttctcccgcattgataaaaaggaactgattaaagaagatctgattaactggctggtcgcgcagaatcgcgaagatgatatcccgaccgtggaaacctttaacaactttaccacgtattttacgggcttccatgaaaaccgtaaaaacatttatagcaaagatgatcatgcgaccgcgattagctttcgcctgattcatgaaaacctgccgaaattttttgataacgtgattagctttaacaaactgaaagaaggttttccggaactgaaatttgataaagtgaaagaagatttagaggtggattatgatctgaaacatgcgtttgagattgaatattttgttaactttgtgacccaggcgggcatagatcagtataactatctgttaggcggtaaaaccctggaagatggcaccaaaaagcagggcatgaatgaacagattaacctgtttaaacagcaacaaacgcgcgataaagcgcgtcagattccgaaactgatcccgctgtttaaacagattttaagcgaaaggaccgaaagtcagagctttattccgaaacagtttgaaagcgatcaggaattgtttgatagcttgcagaaattacataacaactgccaggataaatttaccgtgttgcaacaagcgattctgggcctggcggaggcggatctgaaaaaagtgtttattaaaacctctgatctgaacgcgctgtctaacaccatttttggcaattatagcgtgtttagcgatgcgctgaatctgtataaagaaagtctgaaaaccaaaaaagcgcaggaagcgtttgaaaaactgccagcgcatagcattcatgatctgattcagtatctggaacagtttaactccagcttggatgcggaaaaacagcaaagcaccgataccgtgctgaactattttatcaaaacggatgaactgtattctcgctttattaaaagcaccagcgaagcctttacccaggtgcaaccgttgtttgaactggaagcgctgtccagcaaacgtcgcccgccggaaagcgaagatgagggcgcgaaaggccaggaaggcttcgaacaaatcaaacgtattaaagcgtatctggataccctgatggaagcggtgcactttgcgaaaccgctgtatctggtgaaaggtcgtaaaatgatcgaaggcctcgataaagatcagagcttttacgaagcgtttgaaatggcgtatcaggaattagaaagcttaatcattccgatctataacaaagcgcgtagctatttgtcgcgcaaaccgtttaaagcggataaatttaaaattaactttgataacaacaccctgttaagcggttgggacgcgaacaaagaaaccgccaacgcgtccattctgtttaaaaaagatggcctgtattatctgggtattatgccgaagggtaaaacctttctctttgattattttgtgtcgagcgaagatagcgaaaaactgaaacagcgtcgccagaaaaccgccgaagaagcgctggcgcaggatggcgaaagctattttgaaaaaattcgttataaactgttaccgggcgcgagcaaaatgttaccgaaagtgttttttagcaacaaaaacattggcttttataacccgagcgacgatattctgcgcatccgcaacaccgccagccataccaaaaacggcaccccgcagaaaggccatagcaaagtggaatttaacctgaacgattgccataagatgattgatttttttaaatccagcattcagaaacatccggaatggggatcttttggctttacctttagcgataccagcgattttgaagatatgagcgcgttttatcgcgaagtggaaaatcagggttacgtgattagctttgataaaatcaaagaaacctatatccagagtcaggtggaacagggtaatctgtatctgtttcagatttataacaaagattttagcccgtatagcaaaggcaaaccaaacctgcacaccctgtattggaaagcgttatttgaagaagccaacctgaataacgtggtggcgaaactgaacggtgaagcggaaatcttttttcgtcgtcatagcattaaagcgagcgataaagtggtgcatccggcaaaccaggcgattgataacaaaaatccgcataccgaaaaaacgcagagcacctttgaatatgatctggtgaaagataaacgctatacccaagataaatttttttttcacgtgccgatcagcctcaactttaaagcgcagggcgtgagcaaatttaacgataaagtgaacggcttcctgaaaggcaacccggatgtcaacattattggtattgatcggggcgagcgccatctgctttattttaccgtggtgaatcagaaaggtgaaattctcgttcaggaaagcttaaacaccctgatgagcgataaaggccatgtgaacgattatcagcaaaaactggataaaaaagaacaggagcgtgatgcggcacgtaaatcttggaccacggtggaaaacattaaagaattgaaagaaggctatttaagccatgtggtgcataaactggcgcacctgatcattaaatataacgcgattgtgtgcctggaggacctgaattttggctttaaacgcggtcgctttaaagtggaaaaacaggtttatcagaaatttgaaaaagcgctgattgataaactgaactatctggtgtttaaagaaaaagaattaggtgaagtggggcattatctgaccgcgtatcaactgaccgcgccgttcgaaagctttaaaaaactgggtaaacagtctggcattctgttttacgtcccggcggattatacctccaaaatcgatccgaccacgggcttcgttaactttctggatctgcgctatcagagcgtggaaaaagcgaaacagcttctgtccgattttaacgcgattcgttttaacagcgtgcagaactattttgaatttgaaattgattataaaaaactgaccccgaaacgtaaagtcggcacccaaagtaaatgggttatttgcacctatggcgatgtgcgctatcagaatcgtcgcaatcagaaaggtcattgggaaaccgaagaagtgaacgtgaccgaaaagctgaaagcgttatttgcgagcgatagcaaaacgaccacggttatcgattatgccaacgacgacaacctgattgatgtgattttagaacaggataaagcgagcttttttaaagaattattgtggttactgaaactgaccatgaccctgcgccatagcaaaattaaaagcgaagatgattttattctgtccccggtgaaaaatgaacagggtgaattttatgatagccgtaaagcgggcgaagtttggcctaaagatgcggatgccaacggcgcgtatcatatcgcgctgaaaggcctttggaatttacagcaaattaaccagtgggaaaaaggtaaaaccctgaatttagcgatcaaaaaccaggattggtttagctttatccaggaaaaaccgtatcaggaatgatgaaagcttatgcagatcggtaataaagacgaacaataagacgctgaaaagcgtcttttttcgttttggtcctgttccggcgcgatagtgtgaacatgctatagacttctggtgctacccgactgacaattaatcatccggctcgtataatgctagcaatttctactgttgtagatcattccggaacgttccagcgctgcaatttctactgttgtagatctgatttttcacatgttacctttcaatttctactgttgtagatccgaaaacgtaaagcttcagctgtaatttctactgttgtagatatcatatctggcgttaatggagtttcgtgacgaacaataagtcctccctaacggggggcaatttttattgataacaaaagtaacttcgagcttgtctacctcctagctcgtaaattgcacgctgatagtctcccaattgcgaaggaccaaaacgaaaaaacaccctttcgggtgtcttttctggaatttggtacgcagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaatactaagttgggataactccgtaactgactacgcctttctctagactttacttgaccagatacactgtctttgacacgttgaaggattagagcaatcaaatccaagactggctaagcacgaagcaactcttgagtgttaaaaagttacttcctgtattcgggacgagggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaagtgaagccgacacctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaacaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgatcggaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttccacggtcaagcacaatcagctagcaagatcttggaatagagtcgttgcaccgctttgatttacatgctctccattgcacaacattccggaaggactggcttctctgccatgatcggataatgaaaaacatcagtatgccctgtcatttttctttgggtgtcctcaaataattgccctcacgttatcgtatgtgacgcgctcatctatgctcgaagtattccttgttctcccatcttttaatagaaagtctttaatgaacgtgtcgttacgcagtgtatgaactcttgttttatagggcagactttggcgtggcctaagtgtgttcgataagaaggcaaggacaactagctgacgcgctgtaatacggatattatggcacggttgatacaaacgctgatatcctgatttgctaatgtgcccaacactttagttgagtgccacgttccgactacaagttgcttcaagaggggaatttggatttggcaatagccccccgtttctacctcaagaggcgacgagtattaaccgcgccagctttcggcacaagggccaaagaagattccaatttcttattcccgaataacctccgaatccctgcgggaaaatcaccgaccgaatagcctagaagcaagggggaacagataggtataattagcttaagagagtaccagccgtgacaacaccgtagtaaccacaaacttacgctggggcttctttggcggatttttacagatactaacaaggtgatttgaagtaccttagttgaggatttaaacgcgctatccggtagtctacaaattgggaaataccgttcaaagagggctagaattacttaaaagccttcacaccgcctgcgctatacgcgcccactctcccgtttatccgtccaagcggaagcagggcgaacttccgctaagatattcttacgtgtaacgtagctaagtatcccaaatagctggcgtacgcgttgaacaccgcctagaggatcgggagtcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagttctaatctcttgcccccgcccgtaatagcctccaagtgattcaagatagtaaagggcaagagcttattcggcgttgaaggatagcggactttcggtcaaccacaattccccactcgacaaaaccagccgtgcgaagaactctgaaagtacaagcaacccaagagggctgagcctaaactcagctaattcctaagtgagctaaagactcgaagtgacagctattaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtggatagccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccagaaatcggcatggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagcttagagcttgcgtagcacttagccacaagttaattaacagttgtctggtagtttggcggtattagcgagatcctagaagcaaggcagagttagttctaacctaaagccacaaataagacaggttgccaaagcccgccggaaattaaatcttgctcagttcggtaacggagtttccc

SEQ ID NO: 12 - protein, 1196 aa, synthetic construct
  source 1..1196: note = chimeric STF (STF-V10-[Helix])
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLDSPALTGTPTAPTALRGTNNTQIANTAFVLAAIADVIDASPDALNTLNELAAALGNDPDFATTMTNALAGKQPKNATLTALAGLSTAKNKLPYFAENDAASLTELTQVGRDILAKNSVADVLEYLGAGENSGSATDVMIQLAANDGFKFIGQCPDILTLRTIEPEKNGQRITLRQHTIGTGLGGGVFRAVLDGTGYTDDDGVVIKTAGGSVWLRVNADKVNPFMFGATGVADDTAALQKMLECGRAAELGTNVWKASNLELNNKSCSLSGSGLHVSRIEQISGATGALLTITQDCSLIYLSDCGLYGDGITAGTSGVTMETGNPGGAPSYPFNTAPDVRRDLYISNVHITGFDELGFDYPETNFSVSTHGLFIRNIKKTGAKIGTTDFTWTNLQIDTCGQECLVLDGAGNCRIIGAKLIWAGSENETPYSGLRISNSQNVNMTGVELQDCAYDGLYIKNSTVAISGLNTNRNSASSNLSYHNMVFENSIVTVDGYVCRNYAATSLYDLNSQAGNVRCIGSDSTVLINGIYESEVNSERLMGDNNLIQPYSGDLIINGLKNYYTYTGSVKNNIPTFDGVVTTATYVSAPSILGQGNMLKLTQSNKDKLLFSDKVSRHGCTIGLVLIPSFTGATTMTAFTLGSGYSPSGNSAVMQFIVNSSGVQTIAILLSGDGITQTLTSDLTTEQALASGGVYHFAMGFAPGRLWWSIIDINTGRRIRRAYRQPDLHAAFNSIFNSGTSSITAFSGPLAGDIACEGAGSHVYVGGFSSESDYAASRMYGLFTPVDLDKQYSFRTLNGNI

SEQ ID NO: 13 - DNA, 3588 bp, synthetic construct
  source 1..3588: mol_type = other DNA; note = chimeric STF (STF-V10-[Helix])
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaggcacctctggacagtccggcactgaccggaacgccaacagcaccaaccgcgctcaggggaacaaacaatacccagattgcgaacaccgcttttgtactggccgcgattgcagatgttatcgacgcgtcacctgacgcactgaatacgctgaatgaactggccgcagcgctcgggaatgatccagattttgctaccaccatgactaacgcgcttgcgggtaaacaaccgaagaatgcgacactgacggcgctggcagggctttccacggcgaaaaataaattaccgtattttgcggaaaatgatgccgccagcctgactgaactgactcaggttggcagggatattctggcaaaaaattccgttgcagatgttcttgaataccttggggccggtgagaattcggggagcgctacagacgttatgattcagctggcggcaaatgatggcttcaaattcatcggtcagtgcccagacatcttgaccctgcgtactatcgagccggaaaaaaacggtcagcgtatcaccttacgtcaacatacgattggcactggcttaggcggtggcgttttccgtgcagttctggacggcactggctataccgatgacgacggtgtggtgatcaaaaccgctgggggcagcgtttggctgcgtgtcaacgctgacaaagttaacccgttcatgttcggtgcaaccggagtagcggacgacaccgccgccctgcaaaaaatgctggaatgcggtcgtgcggcggaactggggactaacgtatggaaagcaagcaatctggaactgaacaacaaatcttgctctctgtccggcagtggcctgcacgtttctcgtattgaacagatttccggtgcaaccggagcattgttaaccatcacccaagactgttcgctgatttacctgtccgattgtggcctgtacggcgatggcatcaccgcaggcacgagcggtgttactatggaaacgggtaatccgggtggcgctccgtcttaccctttcaataccgctccggacgttcgtcgtgacctgtacatctctaacgtgcacatcacgggcttcgacgagctgggttttgattatccggaaaccaatttctctgtttcgacgcatggcctcttcatccgtaacatcaaaaaaacgggtgcaaagattggtactacggacttcacttggactaacctgcaaattgatacttgcggtcaggaatgtctggtgctggacggtgcgggtaactgccgtattattggtgcaaaactgatttgggcaggtagcgaaaacgaaacgccatactctggcctgcgtattagcaactctcaaaatgtaaatatgactggcgtagagttacaagactgcgcgtatgatggtttatacatcaagaactctacggttgcaatttcaggcttaaacaccaatcgcaatagcgcatcctctaatctgtcctaccataacatggtattcgaaaattctattgtaactgttgatggttatgtgtgtcgtaactacgcggcgacttcgctgtacgacctgaacagccaagcaggcaacgtccgttgcatcggtagcgacagcaccgttttaatcaacggcatctacgaaagcgaagtcaatagcgagcgcctgatgggtgataacaacctgatccagccgtatagtggtgatctgatcattaacggcctgaaaaattactacacctatactggtagcgtaaaaaacaacattccgaccttcgacggcgttgttactacggcaacctatgtgagcgcaccgtctattctgggtcagggcaatatgctcaaactgacccagtctaataaagacaaactgttatttagcgataaagttagccgtcatggctgtaccatcggcttagttctgattccgtcctttacgggcgcgaccactatgacggcgttcacgctgggtagcggttactctccatccggtaactccgccgtgatgcagttcattgttaacagttccggtgtacaaaccattgcgattttattatccggcgacggtattacccaaaccctgaccagcgatctgaccacggaacaagcactggcgagcggtggcgtgtatcattttgcaatgggttttgcgccgggtcgtttatggtggagcattatcgatattaacacgggcaggcgtattcgtcgcgcctaccgtcagccggatctgcacgcggcgttcaactctatcttcaactccggcacgtcgtctattaccgcatttagcgggccactggcgggcgacattgcttgcgaaggtgcaggtagccatgtatacgttggcggtttttcgtcggaatctgattacgcggctagccgtatgtatggcctgttcactccggtcgatctggacaagcagtatagcttccgtaccctgaacggtaacatt

SEQ ID NO: 14 - protein, 1131 aa, synthetic construct
  source 1..1131: note = chimeric gpJ (1A2)
MGKGSSKGHTPREAKDNLKSTQLLSVIDAISEGPIEGPVDGLKSVLLNSTPVLDTEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVMGNLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNMAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKTWTYNRSNVVMPDDGAPFRYSFSALKDRHNAVEVNWIDPNNGWETATELVEDTQAIARYGRNVTKMDAFGCTSRGQAHRAGLWLIKTELLETQTVDFSVGAEGLRHVPGDVIEICDDDYAGISTGGRVLAVNSQTRTLTLDREITLPSSGTALISLVDGSGNPVSVEVQSVTDGVKVKVSRVPDGVAEYSVWELKLPTLRQRLFRCVSIRENDDGTYAITAVQHVPEKEAIVDNGAHFDGEQSGTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKQIADIRQVETSTRYLGTALYWIAASINIKPGHDYYFYIRSVNTVGKSAFVEAVGRASDDAEGYLDFFKGKITESHLGKELLEKVELTEDNASRLEEFSKEWKDASDKWNAMWAVKIEQTKDGKHYVAGIGLSMEDTEEGKLSQFLVAANRIAFIDPANGNETPMFVAQGNQIFMNDVFLKRLTAPTITSGGNPPAFSLTPDGKLTAKNADISGNVNANSGTLNNVTINENCRVLGKLSANQIEGDLVKTVGKAFPRDSRAPERWPSGTITVRVYDDQPFDRQIVIPAVAFSGAKHEKEHTDIYSSCRLIVRKNGAEIYNRTALDNTLIYSGVIDMPAGHGHMTLEFSVSAWLVNNWYPTASISDLLVVVMKKATAGITIS

SEQ ID NO: 15 - DNA, 3393 bp, synthetic construct
  source 1..3393: mol_type = other DNA; note = chimeric gpJ (1A2)
atgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacataccgcttcacgcaactggcgctggggaactacaggctgacagtccgggcggtaaatgcgtgggggcagcagggcgatccggcgtcggtatcgttccggattgccgcaccggcagcaccgtcgaggattgagctgacgccgggctattttcagataaccgccacgccgcatcttgccgtttatgacccgacggtacagtttgagttctggttctcggaaaagcagattgcggatatcagacaggttgaaaccagcacgcgttatcttggtacggcgctgtactggatagccgccagtatcaatatcaaaccgggccatgattattacttttatatccgcagtgtgaacaccgttggcaaatcggcattcgtggaggccgtcggtcgggcgagcgatgatgcggaaggttacctggattttttcaaaggcaagataaccgaatcccatctcggcaaggagctgctggaaaaagtcgagctgacggaggataacgccagcagactggaggagttttcgaaagagtggaaggatgccagtgataagtggaatgccatgtgggctgtcaaaattgagcagaccaaagacggcaaacattatgtcgcgggtattggcctcagcatggaggacacggaggaaggcaaactgagccagtttctggttgccgccaatcgtatcgcatttattgacccggcaaacgggaatgaaacgccgatgtttgtggcgcagggcaaccagatattcatgaacgacgtgttcctgaagcgcctgacggcccccaccattaccagcggcggcaatcctccggccttttccctgacaccggacggaaagctgaccgctaaaaatgcggatatcagcggtaacgtgaatgcgaactccgggacgctcaacaacgtcacgattaacgagaactgtcgggttctgggaaaattgtccgcgaaccagattgaaggcgatctcgttaaaacagtgggcaaagctttcccccgggactcccgtgcaccggagcggtggccatcaggaaccattaccgtcagggtttatgacgatcagccgtttgaccggcagattgttattccggcggtggcattcagcggcgctaaacatgagaaagagcatactgatatttactcctcatgccgtctgatagtgcggaaaaacggtgctgaaatttataaccgtaccgcgctggataatacgctgatttacagtggcgttattgatatgcctgccggtcacggtcacatgacactggagttttcggtgtcagcatggctggtaaataactggtatcccacagcaagtatcagcgatttgctggttgtggtgatgaagaaagccactgcaggcatcacgattagc

SEQ ID NO: 16 - DNA, 2352 bp, synthetic construct
  source 1..2352: mol_type = other DNA; note = p2.3 pri-ori p1319
gttgtccatatttgctacgtttaaatcaaaactggtgaaactcacccacggatttgcactgacgaaaaacatattttcgataaaccctttagggaaatatgctaagttttcaccgtaacacgccacatcttgactatatatgtgtagaaactgccggaaatcgtcatggtattctgaccagagcgatgagaacgtttcagtttgctcatggaaaacggtgtaacaagggtgaacactatcccatatcaccagttcaccgtctttcattgccatacgaaactccggatgtgcattcatcaggcgggcaagaatgtgaataaaggccggataaaacttgtgcttatttttctttacggtttttaaaaaggccgtaatatccagttgaacggtttggttataggtgcactgagcaactgactggaatgcctcaaaatgttctttacgatgccattgacttatatcaactgtagtatatccagtgatttttttctccattttagattccttagcttgcgaaatctcgataactcaaaaaatagtagtgatcttatttcattatggtgaaagttgtcttacgtgcaacattttcgcaaaaagttggcgctttatcaacactgtccctcctgttcagctactgacggtactgcggaactgactaaagtagtgcgtaacggcaaaagcaccgccggacatcttttgttgcaatggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagccgcgccattactggcatggtggccagtaaggtagataaggtagacaaggggaggcacaactcaaaactttttaaacgagggggtaaaacgcagaccaaaacgatctcaagaagatcatcttattaatcagataaaatatttctagatttcagtgcaatttatctcttcaaatgtagcacgtttagccgaacgccccaaaaagcctcgctttcagcacctgccgtttcctttcttttcagagggtattttaaataaaaacattaagttatgacgaagaagaacggaaacgccttaaaccggaaaattttcataaatagcgaaaacccgcgaggtcgccgccccgtaaccagtcggatcaccggaaagtacctgtaaagtgataatgattatcatcttcatatcacaacgtgcgtaaagggactagtggatactagtattatacctagcactgagcacgggtagcaccagaagtctatagcatgtgcatacctttggtcgaaaaaaaaagcccgcactgtcaggtgcgggcttttttcagtgtttccttgccggattatttgtagagttcatccataccgtgcgtgataccactagcggtaacgaactctaataacaccatgtggtcgcgcttttcgttcggatccttagacagtttagactgggtggacaggtagtggttatccggcagtaaaacaggagcgtctccaatcggagtgttttgttggtaatgatccgcaagctggacgctaccatcttcaacgttatgtcgaattttaaagttagctttgataccgttcttttgtttgtctgcggtgatgtaaacgttatgggagttgaagttatattccagtttgtggcctaaaatattgccgtcctctttgaaatcaatgcctttcagttcaatacgattcaccagagtgtcaccttcaaatttaacctctgcacgggttttatacgtgccatcgtctttgaaagaaatggtgcgctcctgtacataaccttccggcattgcagatttgaagaaatcatgttgcttcatgtggtcagggtaacgagaaaaacactgaacaccataggtcagggtagtcaccagagtaggccacggtactggtaattttccagtagtgcagatgaatttcagggtcagcttaccgttggttgcatcgccttcaccttcaccacgaacactgaatttatgaccgttaacatcgccgtccagttcaactaagatcggaacaacaccagtaaataattcctcacctttactcatggcttcctttctcctctttaatgaaaacttacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaa

SEQ ID NO: 17 - DNA, 2778 bp, synthetic construct
  source 1..2778: mol_type = other DNA; note = p2.8 p15a, p1220
gttgtccatatttgctacgtttaaatcaaaactggtgaaactcacccacggatttgcactgacgaaaaacatattttcgataaaccctttagggaaatatgctaagttttcaccgtaacacgccacatcttgactatatatgtgtagaaactgccggaaatcgtcatggtattctgaccagagcgatgagaacgtttcagtttgctcatggaaaacggtgtaacaagggtgaacactatcccatatcaccagttcaccgtctttcattgccatacgaaactccggatgtgcattcatcaggcgggcaagaatgtgaataaaggccggataaaacttgtgcttatttttctttacggtttttaaaaaggccgtaatatccagttgaacggtttggttataggtgcactgagcaactgactggaatgcctcaaaatgttctttacgatgccattgacttatatcaactgtagtatatccagtgatttttttctccattttagattccttagcttgcgaaatctcgataactcaaaaaatagtagtgatcttatttcattatggtgaaagttgtcttacgtgcaacattttcgcaaaaagttggcgctttatcaacactgtccctcctgttcagctactgacggtactgcggaactgactaaagtagtgcgtaacggcaaaagcaccgccggacatctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggctgagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttactgccgagtttgtctcattccacgcctgacactcagttccgggtaggcagttcgctccaagctggactgtatgcacaaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggtccaaagagttggtagctcagaggaccttcgaaaaaccgccctgcaaggcggttttttcgttttcagagcaagagattacgcgcagaccaaaacgatctcaagccaaaaagcctcgctttcagcacctgccgtttcctttcttttcagagggtattttaaataaaaacattaagttatgacgaagaagaacggaaacgccttaaaccggaaaattttcataaatagcgaaaacccgcgaggtcgccgccccgtaaccagtcggatcaccggaaagtacctgtaaagtgataatgattatcatcttcatatcacaacgtgcgtaaagggactagtggatactagtattatacctagcactgagcacgggtagcaccagaagtctatagcatgtgcatacctttggtcgaaaaaaaaagcccgcactgtcaggtgcgggcttttttcagtgtttccttgccggattatttgtagagttcatccataccgtgcgtgataccactagcggtaacgaactctaataacaccatgtggtcgcgcttttcgttcggatccttagacagtttagactgggtggacaggtagtggttatccggcagtaaaacaggagcgtctccaatcggagtgttttgttggtaatgatccgcaagctggacgctaccatcttcaacgttatgtcgaattttaaagttagctttgataccgttcttttgtttgtctgcggtgatgtaaacgttatgggagttgaagttatattccagtttgtggcctaaaatattgccgtcctctttgaaatcaatgcctttcagttcaatacgattcaccagagtgtcaccttcaaatttaacctctgcacgggttttatacgtgccatcgtctttgaaagaaatggtgcgctcctgtacataaccttccggcattgcagatttgaagaaatcatgttgcttcatgtggtcagggtaacgagaaaaacactgaacaccataggtcagggtagtcaccagagtaggccacggtactggtaattttccagtagtgcagatgaatttcagggtcagcttaccgttggttgcatcgccttcaccttcaccacgaacactgaatttatgaccgttaacatcgccgtccagttcaactaagatcggaacaacaccagtaaataattcctcacctttactcatggcttcctttctcctctttaatgaaaacttacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaa

SEQ ID NO: 18 - DNA, 27 bp, synthetic construct
  source 1..27: mol_type = other DNA; note = primase RBS 1
cttgattaaacgattctggtgtaaaaa

SEQ ID NO: 19 - DNA, 27 bp, synthetic construct
  source 1..27: mol_type = other DNA; note = primase RBS 2
cttgactaaacgattcagtggcaaaaa

SEQ ID NO: 20 - DNA, 27 bp, synthetic construct
  source 1..27: mol_type = other DNA; note = primase RBS 3
cgtgattaaacgatcccgttttcaaaa

SEQ ID NO: 21 - DNA, 27 bp, synthetic construct
  source 1..27: mol_type = other DNA; note = primase RBS 4
cgtgattaaacgatactgttgcaaaaa

SEQ ID NO: 22 - DNA, 27 bp, synthetic construct
  source 1..27: mol_type = other DNA; note = primase RBS 5
cgtgactaaacgatccggggggcaaaa

SEQ ID NO: 23 - DNA, 27 bp, synthetic construct
  source 1..27: mol_type = other DNA; note = primase RBS 6
cttgattaaacgatccaggtttaaaaa

SEQ ID NO: 24 - DNA, 27 bp, synthetic construct
  source 1..27: mol_type = other DNA; note = primase RBS 7
cttgactaaacgatactgtttccaaaa

SEQ ID NO: 25 - DNA, 11789 bp, synthetic construct
  source 1..11789: mol_type = other DNA; note = plasmid lacz6 pri-ori, p1322
ctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtcggaatgacaaatggttccaattattgaacacccttcggggtgtttttttgtttctggtttcccgaggccggccttttgttgcaatggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagccgcgccattactggcatggtggccagtaaggtagataaggtagacaaggggaggcacaactcaaaactttttaaacgagggggtaaaacgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagatcccgatgtacgcgcgcgtggatgatcgagacgaacaataaggcctccctaacggggggccttttttattgataacaaaagtaacttcgagcttgtctacctcctagcaccattattgcaattaataaacaactaacggacaattctacctaacagttttcatatatgacgagcagttaagtgatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagtt

SEQ ID NO: 26 - DNA, 27 bp, synthetic construct
  source 1..27: mol_type = other DNA; note = primase RBS 11
cttgattaaacgattctgtgttcaaaa

SEQ ID NO: 27 - DNA, 12299 bp, synthetic construct
  source 1..12299: mol_type = other DNA; note = plasmid LacZ6 p15a, p780
ctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagatcccgatgtacgcgcgcgtggatgatcgagacgaacaataaggcctccctaacggggggccttttttattgataacaaaagtaacttcgagcttgtctacctcctagcaccattattgcaattaataaacaactaacggacaattctacctaacagttttcatatatgacgagcagttaagtgatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagtt

SEQ ID NO: 28 - DNA, 11782 bp, synthetic construct
  source 1..11782: mol_type = other DNA; note = plasmid LacZ6 pri-ori deltaGAAABCC, p1326
ctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtcggaatgacaaatggttccaattattgaacacccttcggggtgtttttttgtttcccgaggccggccttttgttgcaatggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagcagcgccattactggcatggtggccagtaaggtagataaggtagacaaggggaggcacaactcaaaactttttaaacgagggggtaaaacgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagatcccgatgtacgcgcgcgtggatgatcgagacgaacaataaggcctccctaacggggggccttttttattgataacaaaagtaacttcgagcttgtctacctcctagcaccattattgcaattaataaacaactaacggacaattctacctaacagttttcatatatgacgagcagttaagtgatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagtt

SEQ ID NO: 29 - DNA, 11915 bp, synthetic construct
  source 1..11915: mol_type = other DNA; note = plasmid 4stx pri-ori deltagaaabcc, p1327
ctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaacactgtcggaatgacaaatggttccaattattgaacacccttcggggtgtttttttgtttctggtttcccgaggccggcctttttgcaaggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagcagcgccattactggcatggtggccagtaaggtagataaggtagacaaggggaggcacaactcaaaactttttaaacgagggggtaaaacgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagatcattccggaacgttccagcgctgcaatttctactgttgtagatctgatttttcacatgttacctttcaatttctactgttgtagatccgaaaacgtaaagcttcagctgtaatttctactgttgtagatatcatatctggcgttaatggagtttcgagacgaacaataaggcctccctaacggggggccttttttattgataacaaaagtaacttcgagcttgtctacctcctagcaccattattgcaattaataaacaactaacggacaattctacctaacagttttcatatatgacgagcagttaagtgatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagtt

SEQ ID NO: 30 - DNA, 861 bp, synthetic construct
  source 1..861: mol_type = other DNA; note = beta-lactamase gene
atgagtattcaacatttccgtgtcgcccttattcccttttttgcggcattttgccttcctgtttttgctcacccagaaacgctggtgaaagtaaaagacgctgaggatcagttgggagcccgtgtgggttacatcgagctggatctcaacagcggtaagatccttgagagttttcgccccgaagaacgttttccaatgatgagcacttttaaagttctgctatgtggcgcggtattatcccgtgttgatgccggacaagagcaacttggtcgccgtatacactattctcagaatgacttggttgagtactcaccagttaccgaaaagcatcttacggatggcatgacagtaagagaattatgcagtgctgccataaccatgagtgataacacggcagccaacttacttctgacaacgatcggagggccgaaggagctaaccgcttttttgcacaacatgggggatcatgtaactcgccttgatcgttgggagccggagctgaatgaagccataccaaacgacgagcgtgacaccacgatgcccgcagcaatggcaacaacgttgcgcaaactattaactggcgaactacttactctagcttctcgtcaacaattaatagactggatggaggcggataaagttgcaggcccacttctgcgttcggcccttccggctggctggtttattgctgataaatctggagcaggcgagcgtggatctcgcggtatcattgcagcactggggccagatggtaagccctcccgtatcgtagttatctacacgacggggagtcaggcaactatggatgaacgaaatagacagatcgctgagataggtgcatcactgattaagcattggtaa

(19) United States
(12) Patent Application Publication
(10) Pub. No.: US 2024/0102026 A1
(43) Pub. Date: Mar. 28, 2024

(54) MICROBIOME MODULATION OF A HOST BY DELIVERY OF DNA PAYLOADS WITH MINIMIZED SPREAD

(71) Applicant: Eligo Bioscience, Paris (FR)

(72) Inventors: Jesus FERNANDEZ RODRIGUEZ, Paris (FR); Xavier DUPORTET, Paris (FR)

(21) Appl. No.: 18/348,929

(22) Filed: Jul. 7, 2023

Related U.S. Application Data
(62) Division of application No. 17/565,060, filed on Dec. 29, 2021, now Pat. No. 11,746,352.
Application No. 17/565,060 is a continuation-in-part of application No. 17/138,084, filed on Dec. 30, 2020.
Application No. 17/565,060 is a continuation-in-part of application No. PCT/EP20/88043, filed on Dec. 30, 2020.
(60) Provisional application No. 63/132,090, filed on Dec. 30, 2020.
(60) Provisional application No. 63/132,190, filed on Dec. 30, 2020.
(60) Provisional application No. 62/955,278, filed on Dec. 30, 2019.

(51) Int. Cl. C12N 15/70; C12N 15/113

(52) U.S. Cl. CPC C12N 15/70; C12N 15/113

(57) ABSTRACT

The present invention concerns nucleic acids of interest for modulating the microbiome of a host, to vectors encoding said nucleic acids and to methods for in vivo modulating the microbiome of a subject by delivering said nucleic acid of interest.

### CROSS REFERENCE TO RELATED APPLICATIONS

This application is a divisional of U.S. application Ser. No. 17/565,060 filed on Dec. 29, 2021, which is a continuation-in-part of U.S. application Ser. No. 17/138,084 filed Dec. 30, 2020, and claims the benefit of U.S. application 63/132,090 filed Dec. 30, 2020, U.S. application 63/132,190 filed Dec. 30, 2020, U.S. application 63/137,989 filed Jan. 15, 2021, and is a continuation-in-part of PCT/EP2020/088043 filed Dec. 30, 2020, which claims the benefit of 62/995,278 filed on Dec. 30, 2019 all of which are herein incorporated by reference in their entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Jul. 7, 2023, is named EB2020-06_USDiv.xml and is 120,547 bytes in size.

### TECHNICAL FIELD

The present invention relates to nucleic acids of interest for modulating the microbiome of a host, to vectors encoding said nucleic acids and to methods for modulating the microbiome of a host by delivering said nucleic acids of interest.

### BACKGROUND

Delivery of DNA payloads to express genes of interest in bacterial populations outside of the lab has a lot of applications among which medicine, agriculture, biofueling, cosmetics.

The strategy relies on the delivery of DNA to target bacterial cells in a pure or mixed bacterial population by a viral capsid, by bacterial conjugation or by other methods so that one or several genes of interest will be expressed at a sufficient level to produce a desired effect. The effect can be a direct therapeutic effect on the bacteria itself in or on the host, by killing the bacteria and therefore reducing its colonization level or modifying its ratio compared to other bacteria in the population if multiple species or multiple strains are present; by modifying its genome, by modifying its metabolism or its composition (protein, lipids, sugars, metabolites, RNA, etc.). The effect can also be an indirect effect by leveraging the target bacteria to produce, display or secrete one or multiple molecule(s) such as prophylactic or therapeutic molecule(s) that will have a direct or indirect effect on the host or on other members of the host microbiome.

One of the major concerns with such a strategy is that the exogenous DNA is transferred to progeny cells if the exogenous DNA is stably maintained in the cells in which it is delivered to, or is transferred to other bacteria via other gene transfer mechanism and then stably maintained in these other populations. More generally, the containment of the exogenous DNA payload once delivered in the bacterial populations is a concern.

To solve this issue, the present inventors have herein developed a new strategy that ensures that DNA payloads once delivered in target bacteria cannot replicate in the target bacteria but still express the gene(s) of interest at a level that is enough to exert the expected outcome on the bacteria or on the host, without the need of an antibiotic resistance selection marker on the DNA payload, and without the need of a selection step with an antibiotic.

Plasmids carrying conditional origins of replication have a long history of use by microbiologists as a tool to genetically modify bacterial strains of interest, therefore creating stable genetically modified organisms. They are typically used to select for recombination events between a plasmid carrying such origins and the genome of a bacteria of interest.

Such plasmids carry an antibiotic resistance selection marker and can be introduced into the bacteria by transformation, conjugation or any other method. Because they lack an autonomously replicating origin of replication, only the bacteria that have recombined the plasmid into their genome will stably maintain the selection marker and survive a selection step. The plasmid being stably integrated and maintained in progeny cells, the progeny cells will also be able to survive in presence of the selection marker.

The most commonly used conditional origin of replication is based on the wild-type plasmid R6K and derivatives which belong to the IncX group of replicon, a group commonly found in a variety of bacterial isolates. The replication of these plasmids is dependent on binding of the pir encoded Π initiator protein to the origin of replication. This protein can be expressed from a different replicon (in trans) than the plasmid carrying the R6K origin of replication. In this situation the replication of the R6K on plasmid is conditional on the expression of the pir gene in trans. When delivered to a bacteria of interest, the plasmid will not replicate unless the pir gene is present and expressed.

Similar conditional origins have also been built based on other systems including ColE1 origins (Panayotatos (1984) Nucleic Acids Res. 12:2641-2648) or IncPalpha oriV (Matsumoto-Mashimo et al. (2004) Res. Microbiol. 155:455-461). There are several drawbacks associated with these systems if one would try to build a system with minimal risk of genetically modified material spread in an in vivo setting (human, environment or animal for instance). Notably, such systems are inspired from origins that are almost ubiquitous in nature, such as ColE1 and R6K-type for instance that can be found in many Enterobacteria. Having such an origin on a recombinant plasmid delivered into a microbiome therefore significantly increases the chances not only of recombination with between the recombinant plasmid and wild-type elements within the microbiome, but also of having such plasmid being replicated within this microbiome since the wild-type elements would bring the missing factor necessary for the replication of the plasmid. Additionally, since inducible systems are usually leaky, conditional origins of replication relying on such system have a high chance of being replicated at a basal level—enough to spread in the population—or even at a full replication level if the inducer is present in the target population (for instance, LacI-based origins will be active if lactose is present, which is very often the case in vivo, given modern age diet).

The aim of the present invention is specifically to engineer and efficiently produce vehicles containing a DNA payload that can be transferred to a target bacterial population, not with the purpose of making and selecting recombination events between the DNA payload and the target bacterial genome to create stably genetically modified bacteria that can transfer the modification to progeny cells, but on the opposite with the purpose of limiting and/or preventing the creation of genetically modified progeny cells while still enabling a direct or indirect effect on the bacteria it is delivered into or its host via the efficient expression of genes of interest carried on the DNA payload.

Desired effects to be obtained in targeted bacteria or the host include therapeutic effect, cosmetic effect, bioremediation effect, effects on plant growth or physiology, effects on animal growth or physiology as non limiting examples.

Achieving therapeutic or other type of effect on a target bacteria or its environment with a non-replicative vector is not an obvious development for the simple reason that it can only be achieved if the DNA payload is efficiently delivered to the target bacteria and if it can be expressed to a high enough level and for a sufficient amount of time despite its non-replicative nature. While a replicative plasmid will produce copies of itself, increasing gene dosage, and will be passed down to daughter cells enabling a significant maintenance time in the bacterial population, none of these effects occur with a non-replicative plasmid.

The present inventors here demonstrate, for the first time, that it is possible to obtain an effect in vivo, such as a therapeutic effect, with the delivery of a non-replicative vector to a bacteria.

To this purpose, the present inventors developed a novel conditional origin of replication particularly efficient for this application, that is based on a rarely occurring two-system components to limit recombination events in the target population, the primase and origin of replication of phage-like inducible elements, namely phage-inducible chromosomal islands (PICIs), and they demonstrate for the first time that such type of conditional origin, even with the primase in trans, enables the efficient packaging of the DNA payload into the delivery vehicle, here a phage-derived particle or packaged phagemid.

PICIs, disclosed in Fillol-Salom et al. (2018) The ISME Journal 12:2114-2128 or in Fillol-Salom et al. (2019) Mol. Cell 75:1020-1030 are systems similar to P4-like elements that hijack Myoviridae, with the main difference that, according to current research, they do not modify the size of the capsid to accommodate their genomes. Since lambdoid PICIs are usually 10-13 kb long and the phages they hijack possess genomes close to 50 kb, this means that they are able to insert several copies of their small genome into a large capsid.

According to research, PICIs are able to completely abolish phage production and only lead to the packaging of their genomes. PICIs sense when the lambdoid phage to be hijacked is being induced, they excise from the genome where they reside as prophage-like islands and they replicate their genomes. Replication is based on a single protein, the primase, containing primase and helicase activity, and a short DNA fragment, usually right after the primase gene, that is recognized as an origin of replication by the primase. Additionally, many different PICIs have been described, each one containing different primase-ori pairs.

Fillol-Salom et al. (2018) The ISME Journal 12:2114-2128 specifically discloses PICIs originating in E. coli strain CFT073. In this document, the authors show that the genetic module containing the primase and the ori can function as an independent replication module when inserted in cis in thermosensitive-origin-containing plasmids: at the permissive temperature, the plasmid replicates through the plasmid origin, but when shifted to the non-permissive temperature, the primase and ori module acts as the main source of replication of the plasmid. However, from this observation it is not clear for the skilled person if, even at the non-permissive temperature, replication may have been due to the thermosensitive origin at some degree as it can happen; if the primase and ori can be physically separated (i.e., putting them apart from each other on the same plasmid or having a system in trans) and still enables the replication of the plasmid; and finally, if the ori, that is located right downstream of the primase, is the only element needed for replication or if there is a second element needed and if a specific orientation of the different elements is important, such as in P4, where two elements, the ori and the crr sequence, moreover in a specific orientation, are needed for replication (Flensberg et al. (1987) J. Mol. Biol. 195:439-445).

While other primase-based systems have been developed in which the primase protein is expressed in trans (Ziegelin et al. (2005) J. Bacteriol. 187:3445-3454), it is not known if this type of replication is compatible with phagemid packaging, and even in the case it could be, it would be even less obvious to predict that the packaging would be efficient.

It is indeed also very important that the DNA payload and its vehicle are produced very efficiently in order to be economically viable, which is not an obvious development either. Indeed, some studies have shown that the production titers of phage-derived particles packaging a DNA payload containing a conditional on were reduced by at least 3 logs compared to a DNA payload containing a non-conditional ori, and despite multiple engineering trials, this titer could not get increased.

### SUMMARY OF THE INVENTION

The present invention arises from the unexpected finding that not only a DNA payload devoid of antibiotic resistance marker and autonomously replicative origin of replication can be packaged at high-titer in phage-derived particles but also that these DNA payloads can be efficiently delivered to the target bacteria and that these DNA payloads, while non replicative, can exert the intended effect. In particular, the present inventors also demonstrated for the first time that a non replicative DNA payload expressing a nuclease or an engineered nuclease, such as a base-editor, can result in similar killing or base-editing efficiency as its replicative counterpart.

The present invention thus concerns a method for in vivo modulating the microbiome of a host organism by delivering a nucleic acid of interest into a targeted receiver bacterial cell of said microbiome, said nucleic acid of interest producing a given effect on said targeted receiver bacterial cell,
wherein said method comprises administering, in said host organism, a nucleic acid vector comprising said nucleic acid of interest,
   wherein said vector further comprises a conditional origin of replication which is inactive in the targeted receiver bacterial cell but is active in a donor bacterial cell, and said vector is devoid of antibiotic resistance marker,
thereby delivering said nucleic acid of interest into the targeted receiver bacterial cell, and
wherein, once delivered into said targeted receiver bacterial cell, said nucleic acid of interest produces said given effect on said targeted receiver bacterial cell while said vector is not replicated in said targeted receiver bacterial cell.

The present invention also concerns a method for in vivo modulating the microbiome of a host organism by delivering a nucleic acid of interest into a targeted receiver bacterial cell of said microbiome, said nucleic acid of interest being expressed in said targeted receiver bacterial cell, thereby producing a given effect on said targeted receiver bacterial cell,
wherein said method comprises administering, in said host organism, a nucleic acid vector
comprising said nucleic acid of interest,
   wherein said vector further comprises a conditional origin of replication which is inactive in the targeted receiver bacterial cell but is active in a donor bacterial cell, and said vector is devoid of antibiotic resistance marker,
thereby delivering said nucleic acid of interest into the targeted receiver bacterial cell, and
wherein, once delivered into said targeted receiver bacterial cell, said nucleic acid of interest produces said given effect on said targeted receiver bacterial cell while said vector is not replicated in said targeted receiver bacterial cell.

In a particular embodiment, said given effect on said targeted receiver bacterial cell generates, directly or indirectly, a reaction in said organism hosting said targeted receiver bacterial cell.

### DETAILED DESCRIPTION

### Definitions

As used herein, the term “nucleic acid” refers to a sequence of at least two nucleotides covalently linked together which can be single-stranded or double-stranded or contains portions of both single-stranded and double-stranded sequences. Nucleic acids of the present invention can be naturally occurring, recombinant or synthetic. The nucleic acid can be in the form of a circular sequence or a linear sequence or a combination of both forms. The nucleic acid can be DNA, both genomic or cDNA, or RNA or a combination of both. The nucleic acid may contain any combination of deoxyribonucleotides and ribonucleotides, and any combination of bases, including uracil, adenine, thymine, cytosine, guanine, inosine, xanthine, hypoxanthine, isocytosine, 5-hydroxymethylcytosine and isoguanine. Other examples of modified bases that can be used in the present invention are detailed in Chemical Reviews 2016, 116 (20) 12655-12687. The term “nucleic acid” also encompasses any nucleic acid analogs which may contain other backbones comprising, without limitation, phosphoramide, phosphorothioate, phosphorodithioate, O-methylphosphoroamidite linkage and/or deoxyribonucleotides and ribonucleotides nucleic acids. Any combination of the above features of a nucleic acid is also encompassed by the present invention.

As used herein, the term “peptide” refers both to a short chain of at least 2 amino acids linked between each other and to a part of, a subset of, or a fragment of a protein which part, subset or fragment being not expressed independently from the rest of the protein. In some instances, a peptide is a protein. In some other instances, a peptide is not a protein and peptide only refers to a part, a subset or a fragment of a protein. Preferably, the peptide is from 4, 5, 6, 7, 8, 9, 10, 11, 12, 13, 14, 15 amino acids to 4, 5, 6, 7, 8, 9, 10, 11, 12, 13, 14, 15, 20, 25, 30, 40, 50, 100, 200 amino acids in size.

### Method of In Vivo Modulation

The present invention relates to methods for in vivo modulating the microbiome of a host organism.

By “microbiome” is meant herein the aggregate of all microbiota that reside on or within an organism tissues and biofluids along with the corresponding anatomical sites in which they reside, including, for mammalian organisms, the skin, mammary glands, placenta, seminal fluid, vagina, uterus, ovarian follicles, lung, saliva, oral mucosa, conjunctiva, biliary tract, and gastrointestinal tract, blood, tumors, brain. In a particular embodiment, the microbiome more specifically refers to the bacteria populations forming said microbiota.

By “modulating the microbiome” is meant herein exerting a modifying or controlling influence on the microbiome. In the context of the invention, modulating the microbiome encompases modulating the microbiome function and/or modulating the microbiome composition.

By “modulating the microbiome composition” is meant herein changing the composition of said microbiome, including removing specific species or strains of said microbiome, changing the proportion between different species or strains of said microbiome or replacing specific species or strains of said microbiome by other species or strains. Said modulation of the microbiome composition can be achieved directly or indirectly, typically by modifying said targeted bacterial cell, which can then have an effect, such as a killing effect, on other bacteria of the microbiome, which were not initially targeted by said vector.

By “modulating the microbiome function” is meant herein changing the function of specific species or strains of said microbiome, for example by making specific species or strains express particular molecules, or by making specific species or strains stop expressing particular molecules.

By “host organism” is meant herein any multicellular organism, including any animal or any plant. In a particular embodiment, said host organism is a host subject.

By “host subject” is meant herein any animal (e.g., a primate, e.g., a human) hosting said microbiome. The subject according to the invention is preferably a mammal, even more preferably a human. However, the term “subject” can also refer to non-human animals, in particular mammals such as dogs, cats, horses, cows, pigs, sheep, donkeys, rabbits, ferrets, gerbils, hamsters, chinchillas, rats, mice, guinea pigs and non-human primates, among others, or non-mammals such as poultry, that are in need of treatment.

The human subject according to the invention may be a human at the prenatal stage, a new-born, a child, an infant, an adolescent or an adult at any age.

In the method of the present invention, a nucleic acid of interest is delivered into a targeted receiver bacterial cell of said microbiome or a group of targeted receiver bacterial cells of said microbiome, said nucleic acid of interest being comprised in a vector provided by a donor bacterial cell.

By “donor bacterial cell” is meant herein a bacterium that is capable of hosting a vector comprising a nucleic acid of interest, of producing a vector comprising said nucleic acid of interest and/or which is capable of transferring said vector comprising said nucleic acid to another bacterium. In a particular embodiment, said vector may be a phagemid, and said donor bacterial cell may then be a bacterial cell able to produce said phagemid, more particularly in the form of a packaged phagemid. In an alternative embodiment, said vector may be a plasmid, more particularly a conjugative plasmid, and said donor bacterial cell may then be a bacterium that is capable of transferring said conjugative plasmid to another bacterium, in particular by conjugation.

By “receiver bacterial cell’ is meant herein any bacterium from the host microbiome which is specifically targeted to be delivered with said nucleic acid of interest.

The targeted receiver bacteria can be any bacteria, in particular present in an organism, more particularly in a mammal organism. It can be any commensal, symbiotic or pathogenic bacteria of the microbiota or microbiome.

A microbiome may comprise a variety of endogenous bacterial species, any of which may be targeted in accordance with the present disclosure. In some embodiments, the genus and/or species of targeted receiver bacterial cells may depend on the type of bacteriophages being used for preparing the vector and/or bacterial delivery vehicles. For example, some bacteriophages exhibit tropism for, or preferentially target, specific host species of bacteria. Other bacteriophages do not exhibit such tropism and may be used to target a number of different genus and/or species of endogenous bacterial cells.

Examples of receiver bacterial cells include, without limitation, cells from bacteria of the genus Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Vibrio spp., Bacillus spp., Erysipelothrix spp., Salmonella spp., Streptomyces spp., Streptococcus spp., Staphylococcus spp., Bacteroides spp., Prevotella spp., Clostridium spp., Bifidobacterium spp., Clostridium spp., Brevibacterium spp., Lactococcus spp., Leuconostoc spp., Actinobacillus spp., Selnomonas spp., Shigella spp., Zymonas spp., Mycoplasma spp., Treponema spp., Leuconostoc spp., Corynebacterium spp., Enterococcus spp., Enterobacter spp., Pyrococcus spp., Serratia spp., Morganella spp., Parvimonas spp., Fusobacterium spp., Actinomyces spp., Porphyromonas spp., Propionibacterium spp., Cutibacterium spp., Micrococcus spp., Bartonella spp., Borrelia spp., Brucelia spp., Campylobacter spp., Chlamydophilia spp., Cutibacterium (formerly Propionibacterium) spp., Ehrlichia spp., Haemophilus spp., Leptospira spp., Listeria spp., Mycoplasma spp., Nocardia spp., Rickettsia spp., Ureaplasma spp., and Lactobacillus spp, and a mixture thereof.

Thus, the targeted receiver bacterial cell may be any one or more of the foregoing genus of bacteria.

In an embodiment, the targeted receiver bacteria can be selected from the group consisting of Yersinia spp., Escherichia spp., Klebsiella spp., Acinetobacter spp., Pseudomonas spp., Helicobacter spp., Vibrio spp, Salmonella spp., Streptococcus spp., Staphylococcus spp., Bacteroides spp., Clostridium spp., Shigella spp., Enterococcus spp., Enterobacter spp., Propionibacterium spp., Cutibacterium spp. and Listeria spp.

In some embodiments, targeted receiver bacterial cells of the present disclosure are anaerobic bacterial cells (e.g., cells that do not require oxygen for growth). Anaerobic bacterial cells include facultative anaerobic cells such as but not limited to Escherichia coli, Shewanella oneidensis and Listeria. Anaerobic bacterial cells also include obligate anaerobic cells such as, for example, Bacteroides and Clostridium species. In humans, anaerobic bacteria are most commonly found in the gastrointestinal tract. In some particular embodiments, the targeted receiver bacteria are thus bacteria most commonly found in the gastrointestinal tract.

In some embodiments, the targeted receiver bacterial cells are, without limitation, Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinomycetemcomitans, cyanobacteria, Escherichia coli, Helicobacter pylori, Selenomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Enterococcus faecalis, Bacillus coagulans, Bacillus cereus, Bacillus popillae, Synechocystis strain PCC6803, Bacillus liquefaciens, Pyrococcus abyssi, Selenomonas ruminantium, Lactobacillus hilgardii, Streptococcus ferus, Lactobacillus pentosus, Bacteroides fragilis, Staphylococcus epidermidis, Streptomyces phaechromogenes, Streptomyces ghanaenis, Klebsiella pneumoniae, Enterobacter cloacae, Enterobacter aerogenes, Serratia marcescens, Morganella morganii, Citrobacter freundii, Pseudomonas aeruginosa, Parvimonas micra, Prevotella intermedia, Fusobacterium nucleatum, Prevotella nigrescens, Actinomyces israelii, Porphyromonas endodontalis, Porphyromonas gingivalis Micrococcus luteus, Bacillus megaterium, Aeromonas hydrophila, Aeromonas caviae, Bacillus anthracis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Campylobacter coli, Campylobacter fetus, Chlamydia pneumoniae, Chlamydia trachomatis, Chlamydophila psittaci, Clostridium botulinum, Clostridium difficile, Clostridium perfringens, Clostridium tetani, Corynebacterium diphtheria, Cutibacterium acnes (formerly Propionibacterium acnes), Ehrlichia canis, Ehrlichia chaffeensis, Enterococcus faecium, Francisella tularensis, Haemophilus influenza, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumonia, Neisseria gonorrhoeae, Neisseria meningitides, Nocardia asteroids, Rickettsia rickettsia, Salmonella enteritidis, Salmonella typhi, Salmonella paratyphi, Salmonella typhimurium, Shigella flexneri, Shigella dysenteriae, Staphylococcus saprophyticus, Streptococcus pneumoniae, Streptococcus pyogenes, Streptococcus viridans, Treponema pallidum, Ureaplasma urealyticum, Vibrio cholera, Vibrio parahaemolyticus, Yersinia pestis, Yersinia enterocolitica, Yersinia pseudotuberculosis, Actinobacter baumanii, Pseudomonas aeruginosa, and a mixture thereof. In an embodiment the targeted bacteria of interest are selected from the group consisting of Escherichia coli, Enterococcus faecium, Staphylococcus aureus, Klebsiella pneumoniae, Acinetobacter baumanii, Pseudomonas aeruginosa, Enterobacter cloacae, and Enterobacter aerogenes, and a mixture thereof.

In some embodiments, the targeted bacterial cells are, without limitation, Anaerotruncus, Acetanaerobacterium, Acetitomaculum, Acetivibrio, Anaerococcus, Anaerofilum, Anaerosinus, Anaerostipes, Anaerovorax, Butyrivibrio, Clostridium, Capracoccus, Dehalobacter, Dialister, Dorea, Enterococcus, Ethanoligenens, Faecalibacterium, Fusobacterium, Gracilibacter, Guggenheimella, Hespellia, Lachnobacterium, Lachnospira, Lactobacillus, Leuconostoc, Megamonas, Moryella, Mitsuokella, Oribacterium, Oxobacter, Papillibacter, Proprionispira, Pseudobutyrivibrio, Pseudoramibacter, Roseburia, Ruminococcus, Sarcina, Seinonella, Shuttleworthia, Sporobacter, Sporobacterium, Streptococcus, Subdoligranulum, Syntrophococcus, Thermobacillus, Turibacter, Weisella, Clostridium, Bacteroides, Ruminococcus, Faecalibacterium, Treponema, Phascolarctobacterium, Megasphaera, Faecalibacterium, Bifidobacterium, Lactobacillus, Sutterella, and/or Prevotella.

In other embodiments, the targeted bacteria cells are, without limitation, Achromobacter xylosoxidans, Acidaminococcus fermentans, Acidaminococcus intestini, Acidaminococcus sp., Acinetobacter baumannii, Acinetobacter junii, Acinetobacter lwoffii, Actinobacillus capsulatus, Actinomyces naeslundii, Actinomyces neuii, Actinomyces odontolyticus, Actinomyces radingae, Adlercreutzia equolifaciens, Aeromicrobium massiliense, Aggregatibacter actinomycetemcomitans, Akkermansia muciniphila, Aliagarivorans marinus, Alistipes finegoldii, Alistipes indistinctus, Alistipes inops, Alistipes onderdonkii, Alistipes putredinis, Alistipes senegalensis, Alistipes shahii, Alistipes timonensis, Alloscardovia omnicolens, Anaerobacter polyendosporus, Anaerobaculum hydrogeniformans, Anaerococcus hydrogenalis, Anaerococcus prevotii, Anaerococcus senegalensis, Anaerofustis stercorihominis, Anaerostipes caccae, Anaerostipes hadrus, Anaerotruncus colihominis, Aneurinibacillus aneurinilyticus, Bacillus licheniformis, Bacillus massilioanorexius, Bacillus massiliosenegalensis, Bacillus simplex, Bacillus smithii, Bacillus subtilis, Bacillus thuringiensis, Bacillus timonensis, Bacteroides xylanisolvens, Bacteroides acidifaciens, Bacteroides caccae, Bacteroides capillosus, Bacteroides cellulosilyticus, Bacteroides clarus, Bacteroides coprocola, Bacteroides coprophilus, Bacteroides dorei, Bacteroides eggerthii, Bacteroides faecis, Bacteroides finegoldii, Bacteroides fluxus, Bacteroides fragilis, Bacteroides gallinarum, Bacteroides intestinalis, Bacteroides nordii, Bacteroides oleiciplenus, Bacteroides ovatus, Bacteroides pectinophilus, Bacteroides plebeius, Bacteroides salanitronis, Bacteroides salyersiae, Bacteroides sp., Bacteroides stercoris, Bacteroides thetaiotaomicron, Bacteroides uniformis, Bacteroides vulgatus, Bacteroides xylanisolvens, Bacteroides pectinophilus ATCC, Barnesiella intestinihominis, Bavariicoccus seileri, Bifidobacterium adolescentis, Bifidobacterium angulatum, Bifidobacterium animalis, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Bifidobacterium stercoris, Bilophila wadsworthia, Blautia faecis, Blautia hansenii, Blautia hydrogenotrophica, Blautia luti, Blautia obeum, Blautia producta, Blautia wexlerae, Brachymonas chironomi, Brevibacterium senegalense, Bryantella formatexigens, butyrate-producing bacterium, Butyricicoccus pullicaecorum, Butyricimonas virosa, Butyrivibrio crossotus, Butyrivibrio fibrisolvens, Caldicoprobacter faecalis, Campylobacter concisus, Campylobacter jejuni, Campylobacter upsaliensis, Catenibacterium mitsuokai, Cedecea davisae, Cellulomonas massiliensis, Cetobacterium somerae, Citrobacter braakii, Citrobacter freundii, Citrobacter pasteurii, Citrobacter sp., Citrobacter youngae, Cloacibacillus evryensis, Clostridiales bacterium, Clostridioides difficile, Clostridium asparagiforme, Clostridium bartlettii, Clostridium boliviensis, Clostridium bolteae, Clostridium hathewayi, Clostridium hiranonis, Clostridium hylemonae, Clostridium leptum, Clostridium methylpentosum, Clostridium nexile, Clostridium orbiscindens, Clostridium ramosum, Clostridium scindens, Clostridium sp, Clostridium sp., Clostridium spiroforme, Clostridium sporogenes, Clostridium symbiosum, Collinsella aerofaciens, Collinsella intestinalis, Collinsella stercoris, Collinsella tanakaei, Coprobacillus cateniformis, Coprobacter fastidiosus, Coprococcus catus, Coprococcus comes, Coprococcus eutactus, Corynebacterium ammoniagenes, Corynebacterium amycolatum, Corynebacterium pseudodiphtheriticum, Cutibacterium acnes, Dermabacter hominis, Desulfitobacterium hafniense, Desulfovibrio fairfieldensis, Desulfovibrio piger, Dialister succinatiphilus, Dielma fastidiosa, Dorea formicigenerans, Dorea longicatena, Dysgonomonas capnocytophagoides, Dysgonomonas gadei, Dysgonomonas mossii, Edwardsiella tarda, Eggerthella lenta, Eisenbergiella tayi, Enorma massiliensis, Enterobacter aerogenes, Enterobacter asburiae, Enterobacter cancerogenus, Enterobacter cloacae, Enterobacter massiliensis, Enterococcus casseliflavus, Enterococcus durans, Enterococcus faecalis, Enterococcus faecium, Enterococcus flavescens, Enterococcus gallinarum, Enterococcus sp., Enterovibrio nigricans, Erysipelatoclostridium ramosum, Escherichia coli, Escherichia sp., Eubacterium biforme, Eubacterium dolichum, Eubacterium hallii, Eubacterium limosum, Eubacterium ramulus, Eubacterium rectale, Eubacterium siraeum, Eubacterium ventriosum, Exiguobacterium marinum, Exiguobacterium undae, Faecalibacterium cf, Faecalibacterium prausnitzii, Faecalitalea cylindroides, Ferrimonas balearica, Finegoldia magna, Flavobacterium daejeonense, Flavonifractor plautii, Fusicatenibacter saccharivorans, Fusobacterium gonidiaformans, Fusobacterium mortiferum, Fusobacterium necrophorum, Fusobacterium nucleatum, Fusobacterium periodonticum, Fusobacterium sp., Fusobacterium ulcerans, Fusobacterium varium, Gallibacterium anatis, Gemmiger formicilis, Gordonibacter pamelaeae, Hafnia alvei, Helicobacter bilis, Helicobacter bills, Helicobacter canadensis, Helicobacter canis, Helicobacter cinaedi, Helicobacter macacae, Helicobacter pametensis, Helicobacter pullorum, Helicobacter pylori, Helicobacter rodentium, Helicobacter winghamensis, Herbaspirillum massiliense, Holdemanella biformis, Holdemania fdiformis, Holdemania filiformis, Holdemania massiliensis, Holdemania filiformis, Hungatella hathewayi, Intestinibacter bartlettii, Intestinimonas butyriciproducens, Klebsiella oxytoca, Klebsiella pneumoniae, Kurthia massiliensis, Lachnospira pectinoschiza, Lactobacillus acidophilus, Lactobacillus amylolyticus, Lactobacillus animalis, Lactobacillus antri, Lactobacillus brevis, Lactobacillus buchneri, Lactobacillus casei, Lactobacillus curvatus, Lactobacillus delbrueckii, Lactobacillus fermentum, Lactobacillus gasseri, Lactobacillus helveticus, Lactobacillus hilgardii, Lactobacillus iners, Lactobacillus intestinalis, Lactobacillus johnsonii, Lactobacillus murinus, Lactobacillus paracasei, Lactobacillus plantarum, Lactobacillus reuteri, Lactobacillus rhamnosus, Lactobacillus ruminis, Lactobacillus sakei, Lactobacillus salivarius, Lactobacillus ultunensis, Lactobacillus vaginalis, Lactobacillus plantarum subsp., Leuconostoc mesenteroides, Leuconostoc pseudomesenteroides, Listeria grayi, Listeria innocua, Mannheimia granulomatis, Marvinbryantia formatexigens, Megamonas funiformis, Megamonas hypermegale, Methanobrevibacter smithii, Methanobrevibacter smithii, Micrococcus luteus, Microvirgula aerodenitrificans, Mitsuokella jalaludinii, Mitsuokella multacida, Mollicutes bacterium, Murimonas intestini, Neisseria macacae, Nitriliruptor alkaliphilus, Oceanobacillus massiliensis, Odoribacter laneus, Odoribacter splanchnicus, Ornithobacterium rhinotracheale, Oxalobacter formigenes, Paenibacillus barengoltzii, Paenibacillus chitinolyticus, Paenibacillus lautus, Paenibacillus motobuensis, Paenibacillus senegalensis, Paenisporosarcina quisquiliarum, Parabacteroides distasonis, Parabacteroides goldsteinii, Parabacteroides gordonii, Parabacteroides johnsonii, Parabacteroides merdae, Paraprevotella xylaniphila, Parasutterella excrementihominis, Parvimonas micra, Pediococcus acidilactici, Peptoclostridium difficile, Peptoniphilus harei, Peptoniphilus obesi, Peptoniphilus senegalensis, Peptoniphilus timonensis, Phascolarctobacterium succinatutens, Porphyromonas asaccharolytica, Porphyromonas uenonis, Prevotella baroniae, Prevotella bivia, Prevotella copri, Prevotella dentalis, Prevotella micans, Prevotella multisaccharivorax, Prevotella oralis, Prevotella salivae, Prevotella stercorea, Prevotella veroralis, Propionibacterium acnes, Propionibacterium avidum, Propionibacterium freudenreichii, Propionimicrobium lymphophilum, Proteus mirabilis, Proteus penneri ATCC, Providencia alcalifaciens, Providencia rettgeri, Providencia rustigianii, Providencia stuartii, Pseudoflavonifractor capillosus, Pseudomonas aeruginosa, Pseudomonas luteola, Ralstonia pickettii, Rheinheimera perlucida, Rheinheimera texasensis, Riemerella columbina, Romboutsia lituseburensis, Roseburia faecis, Roseburia intestinalis, Roseburia inulinivorans, Ruminococcus bicirculans, Ruminococcus bromii, Ruminococcus callidus, Ruminococcus champanellensis, Ruminococcus faecis, Ruminococcus gnavus, Ruminococcus lactaris, Ruminococcus obeum, Ruminococcus sp, Ruminococcus sp., Ruminococcus torques, Sarcina ventriculi, Sellimonas intestinalis, Senegalimassilia anaerobia, Shigella sonnei, Slackia piriformis, Staphylococcus epidermidis, Staphylococcus lentus, Staphylococcus nepalensis, Staphylococcus pseudintermedius, Staphylococcus xylosus, Stenotrophomonas maltophilia, Streptococcus agalactiae, Streptococcus anginosus, Streptococcus australis, Streptococcus caballi, Streptococcus castoreus, Streptococcus didelphis, Streptococcus equinus, Streptococcus gordonii, Streptococcus henryi, Streptococcus hyovaginalis, Streptococcus infantarius, Streptococcus infantis, Streptococcus lutetiensis, Streptococcus merionis, Streptococcus mitis, Streptococcus mutans, Streptococcus oralis, Streptococcus ovis, Streptococcus parasanguinis, Streptococcus plurextorum, Streptococcus porci, Streptococcus pyogenes, Streptococcus salivarius, Streptococcus sobrinus, Streptococcus thermophilus, Streptococcus thoraltensis, Streptomyces albus, Subdoligranulum variabile, Succinatimonas hippei, Sutterella parvirubra, Sutterella wadsworthensis, Terrisporobacter glycolicus, Terrisporobacter mayombei, Thalassobacillus devorans, Timonella senegalensis, Turicibacter sanguinis, unknown sp, unknown sp., Varibaculum cambriense, Veillonella atypica, Veillonella dispar, Veillonella parvula, Vibrio cincinnatiensis, Virgibacillus salexigens, and Weissella paramesenteroides.

In other embodiments, the targeted bacteria cells are those commonly found on the skin microbiota and are without limitation Acetobacter farinalis, Acetobacter malorum, Acetobacter orleanensis, Acetobacter sicerae, Achromobacter anxifer, Achromobacter denitrificans, Achromobacter marplatensis, Achromobacter spanius, Achromobacter xylosoxidans subsp. xylosoxidans, Acidovorax konjaci, Acidovorax radicis, Acinetobacter johnsonii, Actinomadura citrea, Actinomadura coerulea, Actinomadura fibrosa, Actinomadura fulvescens, Actinomadura jiaoheensis, Actinomadura luteofluorescens, Actinomadura mexicana, Actinomadura nitritigenes, Actinomadura verrucosispora, Actinomadura yumaensis, Actinomyces odontolyticus, Actinomycetospora atypica, Actinomycetospora corticicola, Actinomycetospora rhizophila, Actinomycetospora rishiriensis, Aeromonas australiensis, Aeromonas bestiarum, Aeromonas bivalvium, Aeromonas encheleia, Aeromonas eucrenophila, Aeromonas hydrophila subsp. hydrophila, Aeromonas piscicola, Aeromonas popoffii, Aeromonas rivuli, Aeromonas salmonicida subsp. pectinolytica, Aeromonas salmonicida subsp. smithia, Amaricoccus kaplicensis, Amaricoccus veronensis, Aminobacter aganoensis, Aminobacter ciceronei, Aminobacter lissarensis, Aminobacter niigataensis, Ancylobacter polymorphus, Anoxybacillus flavithermus subsp. yunnanensis, Aquamicrobium aerolatum, Archangium gephyra, Archangium gephyra, Archangium minus, Archangium violaceum, Arthrobacter viscosus, Bacillus anthracis, Bacillus australimaris, Bacillus drentensis, Bacillus mycoides, Bacillus pseudomycoides, Bacillus pumilus, Bacillus safensis, Bacillus vallismortis, Bosea thiooxidans, Bradyrhizobium huanghuaihaiense, Bradyrhizobium japonicum, Brevundimonas aurantiaca, Brevundimonas intermedia, Burkholderia aspalathi, Burkholderia choica, Burkholderia cordobensis, Burkholderia diffusa, Burkholderia insulsa, Burkholderia rhynchosiae, Burkholderia terrestris, Burkholderia udeis, Buttiauxella gaviniae, Caenimonas terrae, Capnocytophaga gingivalis, Chitinophaga dinghuensis, Chryseobacterium gleum, Chryseobacterium greenlandense, Chryseobacterium jejuense, Chryseobacterium piscium, Chryseobacterium sediminis, Chryseobacterium tructae, Chryseobacterium ureilyticum, Chryseobacterium vietnamense, Corynebacterium accolens, Corynebacterium afermentans subsp. lipophilum, Corynebacterium minutissimum, Corynebacterium sundsvallense, Cupriavidus metallidurans, Cupriavidus nantongensis, Cupriavidus necator, Cupriavidus pampae, Cupriavidus yeoncheonensis, Curtobacterium flaccumfaciens, Devosia epidermidihirudinis, Devosia riboflavina, Devosia riboflavina, Diaphorobacter oryzae, Dietzia psychralcaliphila, Ensifer adhaerens, Ensifer americanus, Enterococcus malodoratus, Enterococcus pseudoavium, Enterococcus viikkiensis, Enterococcus xiangfangensis, Erwinia rhapontici, Falsirhodobacterhalotolerans, Flavobacterium araucananum, Flavobacterium frigidimaris, Gluconobacter frateurii, Gluconobacter thailandicus, Gordonia alkanivorans, Halomonas aquamarina, Halomonas axialensis, Halomonas meridiana, Halomonas olivaria, Halomonas songnenensis, Halomonas variabilis, Herbaspirillum chlorophenolicum, Herbaspirillum frisingense, Herbaspirillum hiltneri, Herbaspirillum huttiense subsp. putei, Herbaspirillum lusitanum, Herminiimonas fonticola, Hydrogenophaga intermedia, Hydrogenophaga pseudoflava, Klebsiella oxytoca, Kosakonia sacchari, Lactobacillus delbrueckii subsp. bulgaricus, Lactobacillus modestisalitolerans, Lactobacillus plantarum subsp. argentoratensis, Lactobacillus xiangfangensis, Lechevalieria roselyniae, Lentzea albida, Lentzea californiensis, Leuconostoc carnosum, Leuconostoc citreum, Leuconostoc gelidum subsp. gasicomitatum, Leuconostoc mesenteroides subsp. suionicum, Luteimonas aestuarii, Lysobacter antibioticus, Lysobacter koreensis, Lysobacter oryzae, Magnetospirillum moscoviense, Marinomonas alcarazii, Marinomonas primoryensis, Massilia aurea, Massilia jejuensis, Massilia kyonggiensis, Massilia timonae, Mesorhizobium acaciae, Mesorhizobium qingshengii, Mesorhizobium shonense, Methylobacterium haplocladii, Methylobacterium platani, Methylobacterium pseudosasicola, Methylobacterium zatmanii, Microbacterium oxydan, Micromonospora chaiyaphumensis, Micromonospora chalcea, Micromonospora citrea, Micromonospora coxensis, Micromonospora echinofusca, Micromonospora halophytica, Micromonospora kangleipakensis, Micromonospora maritima, Micromonospora nigra, Micromonospora purpureochromogene, Micromonospora rhizosphaerae, Micromonospora saelicesensis, Microvirga subterranea, Microvirga zambiensis, Mycobacterium alvei, Mycobacterium avium subsp. silvaticum, Mycobacterium colombiense, Mycobacterium conceptionense, Mycobacterium conceptionense, Mycobacterium farcinogenes, Mycobacterium fortuitum subsp. fortuitum, Mycobacterium goodii, Mycobacterium insubricum, Mycobacterium llatzerense, Mycobacterium neoaurum, Mycobacterium neworleansense, Mycobacterium obuense, Mycobacterium peregrinum, Mycobacterium saopaulense, Mycobacterium septicum, Mycobacterium setense, Mycobacterium smegmatis, Neisseria subflava, Nocardia lijiangensis, Nocardia thailandica, Novosphingobium barchaimii, Novosphingobium lindaniclasticum, Novosphingobium lindaniclasticum, Novosphingobium mathurense, Ochrobactrum pseudogrignonense, Oxalicibacterium solurbis, Paraburkholderia glathei, Paraburkholderia humi, Paraburkholderia phenazinium, Paraburkholderia phytofirmans, Paraburkholderia sordidicola, Paraburkholderia terricola, Paraburkholderia xenovorans, Paracoccus laeviglucosivorans, Patulibacter ginsengiterrae, Polymorphospora rubra, Porphyrobacter colymbi, Prevotella jejuni, Prevotella melaninogenica, Propionibacterium acnes subsp. elongatum, Proteus vulgaris, Providencia rustigianii, Pseudoalteromonas agarivorans, Pseudoalteromonas atlantica, Pseudoalteromonas paragorgicola, Pseudomonas asplenii, Pseudomonas asuensis, Pseudomonas benzenivorans, Pseudomonas cannabina, Pseudomonas cissicola, Pseudomonas congelans, Pseudomonas costantinii, Pseudomonas ficuserectae, Pseudomonas frederiksbergensis, Pseudomonas graminis, Pseudomonas jessenii, Pseudomonas koreensis, Pseudomonas koreensis, Pseudomonas kunmingensis, Pseudomonas marginalis, Pseudomonas mucidolens, Pseudomonas panacis, Pseudomonas plecoglossicida, Pseudomonas poae, Pseudomonas pseudoalcaligenes, Pseudomonas putida, Pseudomonas reinekei, Pseudomonas rhizosphaerae, Pseudomonas seleniipraecipitans, Pseudomonas umsongensis, Pseudomonas zhaodongensis, Pseudonocardia alaniniphila, Pseudonocardia ammonioxydans, Pseudonocardia autotrophica, Pseudonocardia kongjuensis, Pseudonocardia yunnanensis, Pseudorhodoferax soli, Pseudoxanthomonas daejeonensis, Pseudoxanthomonas indica, Pseudoxanthomonas kaohsiungensis, Psychrobacter aquaticus, Psychrobacter arcticus, Psychrobacter celer, Psychrobacter marincola, Psychrobacter nivimaris, Psychrobacter okhotskensis, Psychrobacter okhotskensis, Psychrobacter piscatorii, Psychrobacter pulmonis, Ramlibacter ginsenosidimutans, Rheinheimera japonica, Rheinheimera muenzenbergensis, Rheinheimera soli, Rheinheimera tangshanensis, Rheinheimera texasensis, Rheinheimera tilapiae, Rhizobium alamii, Rhizobium azibense, Rhizobium binae, Rhizobium daejeonense, Rhizobium endophyticum, Rhizobium etli, Rhizobium fabae, Rhizobium freirei, Rhizobium gallicum, Rhizobium loessense, Rhizobium sophoriradicis, Rhizobium taibaishanense, Rhizobium vallis, Rhizobium vignae, Rhizobium vignae, Rhizobium yanglingense, Rhodococcus baikonurensis, Rhodococcus enclensis, Rhodoferax saidenbachensis, Rickettsia canadensis, Rickettsia heilongjiangensis, Rickettsia honei, Rickettsia raoultii, Roseateles aquatilis, Roseateles aquatilis, Salmonella enterica subsp. salamae, Serratia ficaria, Serratia myotis, Serratia vespertilionis, Shewanella aestuarii, Shewanella decolorationis, Sphingobium amiense, Sphingobium baderi, Sphingobium barthaii, Sphingobium chlorophenolicum, Sphingobium cupriresistens, Sphingobium czechense, Sphingobium fuliginis, Sphingobium indicum, Sphingobium indicum, Sphingobium japonicum, Sphingobium lactosutens, Sphingomonas dokdonensis, Sphingomonas pseudosanguinis, Sphingopyxis chilensis, Sphingopyxis fribergensis, Sphingopyxis granuli, Sphingopyxis indica, Sphingopyxis witflariensis, Staphylococcus agnetis, Staphylococcus aureus subsp. aureus, Staphylococcus epidermidis, Staphylococcus hominis subsp. novobiosepticus, Staphylococcus nepalensis, Staphylococcus saprophyticus subsp. bovis, Staphylococcus sciuri subsp. carnaticus, Streptomyces caeruleatus, Streptomyces canarius, Streptomyces capoamus, Streptomyces ciscaucasicus, Streptomyces griseorubiginosus, Streptomyces olivaceoviridis, Streptomyces panaciradicis, Streptomyces phaeopurpureus, Streptomyces pseudovenezuelae, Streptomyces resistomycificus, Tianweitania sediminis, Tsukamurella paurometabola, Variovorax guangxiensis, Vogesella alkaliphila, Xanthomonas arboricola, Xanthomonas axonopodis, Xanthomonas cassavae, Xanthomonas cucurbitae, Xanthomonas cynarae, Xanthomonas euvesicatoria, Xanthomonas fragariae, Xanthomonas gardneri, Xanthomonas perforans, Xanthomonas pisi, Xanthomonas populi, Xanthomonas vasicola, Xenophilus aerolatus, Yersinia nurmii, Abiotrophia defectiva, Acidocella aminolytica, Acinetobacter guangdongensis, Acinetobacter parvus, Acinetobacter radioresistens, Acinetobacter soli, Acinetobacter variabilis, Actinomyces cardiffensis, Actinomyces dentalis, Actinomyces europaeus, Actinomyces gerencseriae, Actinomyces graevenitzii, Actinomyces haliotis, Actinomyces johnsonii, Actinomyces massiliensis, Actinomyces meyeri, Actinomyces meyeri, Actinomyces naeslundii, Actinomyces neuii subsp. anitratus, Actinomyces odontolyticus, Actinomyces oris, Actinomyces turicensis, Actinomycetospora corticicola, Actinotignum schaalii, Aerococcus christensenii, Aerococcus urinae, Aeromicrobium flavum, Aeromicrobium massiliense, Aeromicrobium tamlense, Aeromonas sharmana, Aggregatibacter aphrophilus, Aggregatibactersegnis, Agrococcus baldri, Albibactermethylovorans, Alcaligenes faecalis subsp. faecalis, Algoriphagus ratkowskyi, Alkalibacterium olivapovliticus, Alkalibacterium pelagium, Alkalibacterium pelagium, Alloprevotella rava, Alsobacter metallidurans, Amaricoccus kaplicensis, Amaricoccus veronensis, Anaerococcus hydrogenalis, Anaerococcus lactolyticus, Anaerococcus murdochii, Anaerococcus octavius, Anaerococcus prevotii, Anaerococcus vaginalis, Aquabacterium citratiphilum, Aquabacterium olei, Aquabacterium olei, Aquabacterium parvum, Aquincola tertiaricarbonis, Arcobacter venerupis, Arsenicicoccus bolidensis, Arthrobacter russicus, Asticcacaulis excentricus, Atopobium deltae, Atopobium parvulum, Atopobium rimae, Atopobium vaginae, Aureimonas altamirensis, Aureimonas rubiginis, Azospira oryzae, Azospirillum oryzae, Bacillus circulans, Bacillus drentensis, Bacillus fastidiosus, Bacillus lehensis, Bacillus oceanisediminis, Bacillus rhizosphaerae, Bacteriovorax stolpii, Bacteroides coagulans, Bacteroides dorei, Bacteroides fragilis, Bacteroides ovatus, Bacteroides stercoris, Bacteroides uniformis, Bacteroides vulgatus, Bdellovibrio bacteriovorus, Bdellovibrio exovorus, Belnapia moabensis, Belnapia soli, Blautia hansenii, Blautia obeum, Blautia wexlerae, Bosea lathyri, Brachybacterium fresconis, Brachybacterium muris, Brevibacterium ammoniilyticum, Brevibacterium casei, Brevibacterium epidermidis, Brevibacterium iodinum, Brevibacterium luteolum, Brevibacterium paucivorans, Brevibacterium pityocampae, Brevibacterium sanguinis, Brevundimonas albigilva, Brevundimonas diminuta, Brevundimonas vancanneytii, Caenimonas terrae, Calidifontibacter indicus, Campylobacter concisus, Campylobacter gracilis, Campylobacter hominis, Campylobacter rectus, Campylobacter showae, Campylobacter ureolyticus, Capnocytophaga gingivalis, Capnocytophaga leadbetteri, Capnocytophaga ochracea, Capnocytophaga sputigena, Cardiobacterium hominis, Cardiobacterium valvarum, Carnobacterium divergens, Catonella morbi, Caulobacter henricii, Cavicella subterranea, Cellulomonas xylanilytica, Cellvibrio vulgaris, Chitinimonas taiwanensis, Chryseobacterium arachidis, Chryseobacterium daecheongense, Chryseobacterium formosense, Chryseobacterium formosense, Chryseobacterium greenlandense, Chryseobacterium indologenes, Chryseobacterium piscium, Chryseobacterium rigui, Chryseobacterium solani, Chryseobacterium taklimakanense, Chryseobacterium ureilyticum, Chryseobacterium ureilyticum, Chryseobacterium zeae, Chryseomicrobium aureum, Cloacibacterium haliotis, Cloacibacterium normanense, Cloacibacterium normanense, Collinsella aerofaciens, Comamonas denitrificans, Comamonas terrigena, Corynebacterium accolens, Corynebacterium afermentans subsp. lipophilum, Corynebacterium ammoniagenes, Corynebacterium amycolatum, Corynebacterium aurimucosum, Corynebacterium aurimucosum, Corynebacterium coyleae, Corynebacterium durum, Corynebacterium freiburgense, Corynebacterium glaucum, Corynebacterium glyciniphilum, Corynebacterium imitans, Corynebacterium jeikeium, Corynebacterium jeikeium, Corynebacterium kroppenstedtii, Corynebacterium lipophiloflavum, Corynebacterium massiliense, Corynebacterium mastitidis, Corynebacterium matruchotii, Corynebacterium minutissimum, Corynebacterium mucifaciens, Corynebacterium mustelae, Corynebacterium mycetoides, Corynebacterium pyruviciproducens, Corynebacterium simulans, Corynebacterium singulare, Corynebacterium sputi, Corynebacterium suicordis, Corynebacterium tuberculostearicum, Corynebacterium tuberculostearicum, Corynebacterium ureicelerivorans, Corynebacterium variabile, Couchioplanes caeruleus subsp. caeruleus, Cupriavidus metallidurans, Curtobacterium herbarum, Dechloromonas agitata, Deinococcus actinosclerus, Deinococcus antarcticus, Deinococcus caeni, Deinococcus ficus, Deinococcus geothermalis, Deinococcus radiodurans, Deinococcus wulumuqiensis, Deinococcus xinjiangensis, Dermabacter hominis, Dermabacter vaginalis, Dermacoccus nishinomiyaensis, Desemzia incerta, Desertibacter roseus, Dialister invisus, Dialister micraerophilus, Dialister propionicifaciens, Dietzia aurantiaca, Dietzia cercidiphylli, Dietzia timorensis, Dietzia timorensis, Dokdonella koreensis, Dokdonella koreensis, Dolosigranulum pigrum, Eikenella corrodens, Elizabethkingia miricola, Elstera litoralis, Empedobacter brevis, Enhydrobacter aerosaccus, Enterobacter xiangfangensis, Enterococcus aquimarinus, Enterococcus faecalis, Enterococcus olivae, Erwinia rhapontici, Eubacterium eligens, Eubacterium infirmum, Eubacterium rectale, Eubacterium saphenum, Eubacterium sulci, Exiguobacterium mexicanum, Facklamia tabacinasalis, Falsirhodobacter halotolerans, Finegoldia magna, Flavobacterium cutihirudinis, Flavobacterium lindanitolerans, Flavobacterium resistens, Friedmanniella capsulata, Fusobacterium nucleatum subsp. polymorphum, Gemella haemolysans, Gemella morbillorum, Gemella palaticanis, Gemella sanguinis, Gemmobacter aquaticus, Gemmobacter caeni, Gordonia jinhuaensis, Gordonia kroppenstedtii, Gordonia polyisoprenivorans, Gordonia polyisoprenivorans, Granulicatella adiacens, Granulicatella elegans, Haemophilus parainfluenzae, Haemophilus sputorum, Halomonas sulfidaeris, Herpetosiphon aurantiacus, Hydrocarboniphaga effusa, Idiomarina maris, Janibacter anophelis, Janibacter hoylei, Janibacter indicus, Janibacter limosus, Janibacter melonis, Jeotgalicoccus halophilus, Jonquetella anthropi, Kaistia geumhonensis, Kingella denitrificans, Kingella oralis, Klebsiella oxytoca, Knoellia aerolata, Knoellia locipacati, Kocuria atrinae, Kocuria carniphila, Kocuria kristinae, Kocuria palustris, Kocuria turfanensis, Lachnoanaerobaculum saburreum, Lachnoanaerobaculum saburreum, Lactobacillus crispatus, Lactobacillus iners, Lactococcus lactis subsp. lactis, Lactococcus lactis subsp. lactis, Lactococcus piscium, Lapillicoccus jejuensis, Lautropia mirabilis, Legionella beliardensis, Leptotrichia buccalis, Leptotrichia goodfellowii, Leptotrichia hofstadii, Leptotrichia hongkongensis, Leptotrichia shahii, Leptotrichia trevisanii, Leptotrichia wadei, Luteimonas terricola, Lysinibacillus fusiformis, Lysobacter spongiicola, Lysobacter xinjiangensis, Macrococcus caseolyticus, Marmoricola pocheonensis, Marmoricola scoriae, Massilia alkalitolerans, Massilia alkalitolerans, Massilia aurea, Massilia plicata, Massilia timonae, Megamonas rupellensis, Meiothermus silvanus, Methylobacterium dankookense, Methylobacterium goesingense, Methylobacterium goesingense, Methylobacterium isbiliense, Methylobacterium jeotgali, Methylobacterium oxalidis, Methylobacterium platani, Methylobacterium pseudosasicola, Methyloversatilis universalis, Microbacterium foliorum, Microbacterium hydrothermale, Microbacterium hydrothermale, Microbacterium lacticum, Microbacterium lacticum, Microbacterium laevaniformans, Microbacterium paludicola, Microbacterium petrolearium, Microbacterium phyllosphaerae, Microbacterium resistens, Micrococcus antarcticus, Micrococcus cohnii, Micrococcus flavus, Micrococcus lylae, Micrococcus terreus, Microlunatus aurantiacus, Micropruina glycogenica, Microvirga aerilata, Microvirga aerilata, Microvirga subterranea, Microvirga vignae, Microvirga zambiensis, Microvirgula aerodenitrificans, Mogibacterium timidum, Moraxella atlantae, Moraxella catarrhalis, Morganella morganii subsp. morganii, Morganella psychrotolerans, Murdochiella asaccharolytica, Mycobacterium asiaticum, Mycobacterium chubuense, Mycobacterium crocinum, Mycobacterium gadium, Mycobacterium holsaticum, Mycobacterium iranicum, Mycobacterium longobardum, Mycobacterium neoaurum, Mycobacterium neoaurum, Mycobacterium obuense, Negativicoccus succinicivorans, Neisseria bacilliformis, Neisseria oralis, Neisseria sicca, Neisseria subflava, Nesterenkonia lacusekhoensis, Nesterenkonia rhizosphaerae, Nevskia persephonica, Nevskia ramosa, Niabella yanshanensis, Niveibacterium umoris, Nocardia niwae, Nocardia thailandica, Nocardioides agariphilus, Nocardioides dilutus, Nocardioides ganghwensis, Nocardioides hwasunensis, Nocardioides nanhaiensis, Nocardioides sediminis, Nosocomiicoccus ampullae, Noviherbaspirillum malthae, Novosphingobium lindaniclasticum, Novosphingobium rosa, Ochrobactrum rhizosphaerae, Olsenella uli, Ornithinimicrobium murale, Ornithinimicrobium tianjinense, Oryzobacterterrae, Ottowia beijingensis, Paenalcaligenes suwonensis, Paenibacillus agaridevorans, Paenibacillus phoenicis, Paenibacillus xylanexedens, Paludibacterium yongneupense, Pantoea cypripedii, Parabacteroides distasonis, Paraburkholderia andropogonis, Paracoccus alcaliphilus, Paracoccus angustae, Paracoccus kocurii, Paracoccus laeviglucosivorans, Paracoccus sediminis, Paracoccus sphaerophysae, Paracoccus yeei, Parvimonas micra, Parviterribacter multiflagellatus, Patulibacter ginsengiterrae, Pedobacter aquatilis, Pedobacter ginsengisoli, Pedobacter xixiisoli, Peptococcus niger, Peptoniphilus coxii, Peptoniphilus gorbachii, Peptoniphilus harei, Peptoniphilus koenoeneniae, Peptoniphilus lacrimalis, Peptostreptococcus anaerobius, Peptostreptococcus stomatis, Phascolarctobacterium faecium, Phenylobacterium haematophilum, Phenylobacterium kunshanense, Pluralibacter gergoviae, Polymorphobacter multimanifer, Porphyromonas bennonis, Porphyromonas endodontalis, Porphyromonas gingivalis, Porphyromonas gingivicanis, Porphyromonas pasteri, Porphyromonas pogonae, Porphyromonas somerae, Povalibacter uvarum, Prevotella aurantiaca, Prevotella baroniae, Prevotella bivia, Prevotella buccae, Prevotella buccalis, Prevotella copri, Prevotella corporis, Prevotella denticola, Prevotella enoeca, Prevotella histicola, Prevotella intermedia, Prevotella jejuni, Prevotella jejuni, Prevotella maculosa, Prevotella melaninogenica, Prevotella melaninogenica, Prevotella micans, Prevotella multiformis, Prevotella nanceiensis, Prevotella nigrescens, Prevotella oris, Prevotella oulorum, Prevotella pallens, Prevotella pleuritidis, Prevotella saccharolytica, Prevotella salivae, Prevotella shahii, Prevotella timonensis, Prevotella veroralis, Propionibacterium acidifaciens, Propionibacterium acnes subsp. acnes, Propionibacterium acnes subsp. acnes, Propionibacterium acnes subsp. elongatum, Propionibacterium granulosum, Propionimicrobium lymphophilum, Propionispira arcuata, Pseudokineococcus lusitanus, Pseudomonas aeruginosa, Pseudomonas chengduensis, Pseudonocardia benzenivorans, Pseudorhodoplanes sinuspersici, Psychrobacter sanguinis, Ramlibacter ginsenosidimutans, Rheinheimera aquimaris, Rhizobium alvei, Rhizobium daejeonense, Rhizobium larrymoorei, Rhizobium rhizoryzae, Rhizobium soli, Rhizobium taibaishanense, Rhizobium vignae, Rhodanobacter glycinis, Rhodobacter veldkampii, Rhodococcus enclensis, Rhodococcus fascians, Rhodococcus fascians, Rhodovarius lipocyclicus, Rivicola pingtungensis, Roseburia inulinivorans, Rosenbergiella nectarea, Roseomonas aerilata, Roseomonas aquatica, Roseomonas mucosa, Roseomonas rosea, Roseomonas vinacea, Rothia aeria, Rothia amarae, Rothia dentocariosa, Rothia endophytica, Rothia mucilaginosa, Rothia nasimurium, Rubellimicrobium mesophilum, Rubellimicrobium roseum, Rubrobacter bracarensis, Rudaea cellulosilytica, Ruminococcus gnavus, Runella zeae, Saccharopolyspora rectivirgula, Salinicoccus qingdaonensis, Scardovia wiggsiae, Sediminibacterium ginsengisoli, Selenomonas artemidis, Selenomonas infelix, Selenomonas noxia, Selenomonas sputigena, Shewanella aestuarii, Shuttleworthia satelles, Simonsiella muelleri, Skermanella aerolata, Skermanella stibiiresistens, Slackia exigua, Smaragdicoccus niigatensis, Sneathia sanguinegens, Solirubrobacter soli, Sphingobacterium caeni, Sphingobacterium daejeonense, Sphingobacterium hotanense, Sphingobacterium kyonggiense, Sphingobacterium multivorum, Sphingobacterium nematocida, Sphingobacterium spiritivorum, Sphingobium amiense, Sphingobium indicum, Sphingobium lactosutens, Sphingobium subterraneum, Sphingomonas abaci, Sphingomonas aestuarii, Sphingomonas canadensis, Sphingomonas daechungensis, Sphingomonas dokdonensis, Sphingomonas echinoides, Sphingomonas fonticola, Sphingomonas fonticola, Sphingomonas formosensis, Sphingomonas gei, Sphingomonas hankookensis, Sphingomonas hankookensis, Sphingomonas koreensis, Sphingomonas kyeonggiensis, Sphingomonas laterariae, Sphingomonas mucosissima, Sphingomonas oligophenolica, Sphingomonas pseudosanguinis, Sphingomonas sediminicola, Sphingomonas yantingensis, Sphingomonas yunnanensis, Sphingopyxis indica, Spirosoma rigui, Sporacetigenium mesophilum, Sporocytophaga myxococcoides, Staphylococcus auricularis, Staphylococcus epidermidis, Staphylococcus epidermidis, Staphylococcus hominis subsp. novobiosepticus, Staphylococcus lugdunensis, Staphylococcus pettenkoferi, Stenotrophomonas koreensis, Stenotrophomonas rhizophila, Stenotrophomonas rhizophila, Streptococcus agalactiae, Streptococcus canis, Streptococcus cristatus, Streptococcus gordonii, Streptococcus infantis, Streptococcus intermedius, Streptococcus mutans, Streptococcus oligofermentans, Streptococcus oralis, Streptococcus sanguinis, Streptomyces iconiensis, Streptomyces yanglinensis, Tabrizicola aquatica, Tahibacter caeni, Tannerella forsythia, Tepidicella xavieri, Tepidimonas fonticaldi, Terracoccus luteus, Tessaracoccus flavescens, Thermus thermophilus, Tianweitania sediminis, Tianweitania sediminis, Treponema amylovorum, Treponema denticola, Treponema lecithinolyticum, Treponema medium, Turicella otitidis, Turicibacter sanguinis, Undibacterium oligocarboniphilum, Undibacterium squillarum, Vagococcus salmoninarum, Varibaculum cambriense, Vibrio metschnikovii, Xanthobacter tagetidis, Xenophilus aerolatus, Xenophilus arseniciresistens, Yimella lutea, Zimmermannella alba, Zimmermannella bifida and Zoogloea caeni.

In other embodiments, the targeted bacteria cells are those commonly found in the vaginal microbiota and are, without limitation, Acinetobacter antiviralis, Acinetobacter baumannii, Acinetobacter calcoaceticus, Acinetobacter johnsonii, Actinobaculum massiliense, Actinobaculum schaalii, Actinomyces europaeus, Actinomyces graevenitzii, Actinomyces israelii, Actinomyces meyeri, Actinomyces naeslundii, Actinomyces neuii, Actinomyces odontolyticus, Actinomyces turicensis, Actinomyces urogenitalis, Actinomyces viscosus, Aerococcus christensenii, Aerococcus urinae, Aerococcus viridans, Aeromonas encheleia, Aeromonas salmonicida, Afipia massiliensis, Agrobacterium tumefaciens, Algoriphagus aquatilis, Aliivibrio wodanis, Alistipes finegoldii, Alloiococcus otitis, Alloprevotella tannerae, Alloscardovia omnicolens, Altererythrobacter epoxidivorans, Ammoniphilus oxalaticus, Amnibacterium kyonggiense, Anaerococcus hydrogenalis, Anaerococcus lactolyticus, Anaerococcus murdochii, Anaerococcus obesiensis, Anaerococcus prevotii, Anaerococcus tetradius, Anaerococcus vaginalis, Anaeroglobus geminatus, Anoxybacillus pushchinoensis, Aquabacterium parvum, Arcanobacterium phocae, Arthrobacter aurescens, Asticcacaulis excentricus, Atopobium minutum, Atopobium parvulum, Atopobium rimae, Atopobium vaginae, Avibacterium gallinarum, Bacillus acidicola, Bacillus atrophaeus, Bacillus cereus, Bacillus cibi, Bacillus coahuilensis, Bacillus gaemokensis, Bacillus methanolicus, Bacillus oleronius, Bacillus pumilus, Bacillus shackletonii, Bacillus sporothermodurans, Bacillus subtilis, Bacillus wakoensis, Bacillus weihenstephanensis, Bacteroides barnesiae, Bacteroides coagulans, Bacteroides dorei, Bacteroides faecis, Bacteroides forsythus, Bacteroides fragilis, Bacteroides nordii, Bacteroides ovatus, Bacteroides salyersiae, Bacteroides stercoris, Bacteroides uniformis, Bacteroides vulgatus, Bacteroides xylanisolvens, Bacteroides zoogleoformans, Barnesiella viscericola, Bhargavaea cecembensis, Bifidobacterium adolescentis, Bifidobacterium bifidum, Bifidobacterium breve, Bifidobacterium dentium, Bifidobacterium logum subsp. infantis, Bifidobacterium longum, Bifidobacterium pseudocatenulatum, Bifidobacterium scardovii, Bilophila wadsworthia, Blautia hydrogenotrophica, Blautia obeum, Blautia producta, Brachybacterium faecium, Bradyrhizobium japonicum, Brevibacterium mcbrellneri, Brevibacterium otitidis, Brevibacterium paucivorans, Bulleidia extructa, Burkholderia fungorum, Burkholderia phenoliruptix, Caldicellulosiruptor saccharolyticus, Caldimonas taiwanensis, Campylobacter gracilis, Campylobacter hominis, Campylobacter sputorum, Campylobacter ureolyticus, Capnocytophaga ochracea, Cardiobacterium hominis, Catonella morbi, Chlamydia trachomatis, Chlamydophila abortus, Chondromyces robustus, Chryseobacterium aquaticum, Citrobacter youngae, Cloacibacterium normanense, Clostridium cavendishii, Clostridium colicanis, Clostridium jejuense, Clostridium perfringens, Clostridium ramosum, Clostridium sordellii, Clostridium viride, Comamonas terrigena, Corynebacterium accolens, Corynebacterium appendicis, Corynebacterium coyleae, Corynebacterium glucuronolyticum, Corynebacterium glutamicum, Corynebacterium jeikeium, Corynebacterium kroppenstedtii, Corynebacterium lipophiloflavum, Corynebacterium minutissimum, Corynebacterium mucifaciens, Corynebacterium nuruki, Corynebacterium pseudogenitalium, Corynebacterium pyruviciproducens, Corynebacterium singulare, Corynebacterium striatum, Corynebacterium tuberculostearicum, Corynebacterium xerosis, Cryobacterium psychrophilum, Curtobacterium flaccumfaciens, Cutibacterium acnes, Cutibacterium avidum, Cytophaga xylanolytica, Deinococcus radiophilus, Delftia tsuruhatensis, Desulfovibrio desulfuricans, Dialister invisus, Dialister micraerophilus, Dialister pneumosintes, Dialister propionicifaciens, Dickeya chrysanthemi, Dorea longicatena, Eggerthella lenta, Eggerthia catenaformis, Eikenella corrodens, Enhydrobacter aerosaccus, Enterobacter asburiae, Enterobacter cloacae, Enterococcus avium, Enterococcus durans, Enterococcus faecalis, Enterococcus faecium, Enterococcus hirae, Erwinia persicina, Erwinia rhapontici, Erwinia toletana, Escherichia coli, Escherichia fergusonii, Eubacterium brachy, Eubacterium eligens, Eubacterium nodatum, Eubacterium rectale, Eubacterium saphenum, Eubacterium siraeum, Eubacterium sulci, Eubacterium yurii, Exiguobacterium acetylicum, Facklamia ignava, Faecalibacterium prausnitzii, Filifactor alocis, Finegoldia magna, Fusobacterium gonidiaformans, Fusobacterium nucleatum, Fusobacterium periodonticum, Gardnerella vaginalis, Gemella asaccharolytica, Gemella bergeri, Gemella haemolysans, Gemella sanguinis, Geobacillus stearothermophilus, Geobacillus thermocatenulatus, Geobacillus thermoglucosidasius, Geobacter grbiciae, Granulicatella elegans, Haemophilus ducreyi, Haemophilus haemolyticus, Haemophilus parahaemolyticus, Haemophilus parainfluenzae, Hafnia alvei, Halomonas meridiana, Halomonas phoceae, Halomonas venusta, Herbaspirillum seropedicae, Janthinobacterium lividum, Jonquetella anthropi, Klebsiella granulomatis, Klebsiella oxytoca, Klebsiella pneumoniae, Lactobacillus acidophilus, Lactobacillus amylovorus, Lactobacillus brevis, Lactobacillus coleohominis, Lactobacillus crispatus, Lactobacillus curvatus, Lactobacillus delbrueckii, Lactobacillus fermentum, Lactobacillus gasseri, Lactobacillus helveticus, Lactobacillus iners, Lactobacillus jensenii, Lactobacillus johnsonii, Lactobacillus kalixensis, Lactobacillus kefiranofaciens, Lactobacillus kimchicus, Lactobacillus kitasatonis, Lactobacillus mucosae, Lactobacillus panis, Lactobacillus paracasei, Lactobacillus plantarum, Lactobacillus pontis, Lactobacillus reuteri, Lactobacillus rhamnosus, Lactobacillus salivarius, Lactobacillus ultunensis, Lactobacillus vaginalis, Lactococcus lactis, Leptotrichia buccalis, Leuconostoc carnosum, Leuconostoc citreum, Leuconostoc garlicum, Leuconostoc lactis, Leuconostoc mesenteroides, Lysinimonas kribbensis, Mageeibacillus indolicus, Maribacter orientalis, Marinomonas protea, Marinospirillum insulare, Massilia timonae, Megasphaera elsdenii, Megasphaera micronuciformis, Mesorhizobium amorphae, Methylobacterium radiotolerans, Methylotenera versatilis, Microbacterium halophilum, Micrococcus luteus, Microterricola viridarii, Mobiluncus curtisii, Mobiluncus mulieris, Mogibacterium timidum, Moorella glycerini, Moraxella osloensis, Morganella morganii, Moryella indoligenes, Murdochiella asaccharolytica, Mycoplasma alvi, Mycoplasma genitalium, Mycoplasma hominis, Mycoplasma muris, Mycoplasma salivarium, Negativicoccus succinicivorans, Neisseria flava, Neisseria gonorrhoeae, Neisseria mucosa, Neisseria subflava, Nevskia ramosa, Nevskia soli, Nitriliruptor alkaliphilus, Odoribacter splanchnicus, Oligella urethralis, Olsenella uli, Paenibacillus amylolyticus, Paenibacillus humicus, Paenibacillus pabuli, Paenibacillus pasadenensis, Paenibacillus pini, Paenibacillus validus, Pantoea agglomerans, Parabacteroides merdae, Paraburkholderia caryophylli, Paracoccus yeei, Parastreptomyces abscessus, Parvimonas micra, Pectobacterium betavasculorum, Pectobacterium carotovorum, Pediococcus acidilactici, Pediococcus ethanolidurans, Pedobacter alluvionis, Pedobacter wanjuense, Pelomonas aquatica, Peptococcus niger, Peptoniphilus asaccharolyticus, Peptoniphilus gorbachii, Peptoniphilus harei, Peptoniphilus indolicus, Peptoniphilus lacrimalis, Peptoniphilus massiliensis, Peptostreptococcus anaerobius, Peptostreptococcus massiliae, Peptostreptococcus stomatis, Photobacterium angustum, Photobacterium frigidiphilum, Photobacterium phosphoreum, Porphyromonas asaccharolytica, Porphyromonas bennonis, Porphyromonas catoniae, Porphyromonas endodontalis, Porphyromonas gingivalis, Porphyromonas somerae, Porphyromonas uenonis, Prevotella amnii, Prevotella baroniae, Prevotella bergensis, Prevotella bivia, Prevotella buccae, Prevotella buccalis, Prevotella colorans, Prevotella copri, Prevotella corporis, Prevotella dentalis, Prevotella denticola, Prevotella disiens, Prevotella intermedia, Prevotella loescheii, Prevotella marshii, Prevotella melaninogenica, Prevotella micans, Prevotella nigrescens, Prevotella oris, Prevotella pleuritidis, Prevotella ruminicola, Prevotella shahii, Prevotella stercorea, Prevotella timonensis, Prevotella veroralis, Propionimicrobium lymphophilum, Proteus mirabilis, Pseudomonas abietaniphila, Pseudomonas aeruginosa, Pseudomonas amygdali, Pseudomonas azotoformans, Pseudomonas chlororaphis, Pseudomonas cuatrocienegasensis, Pseudomonas fluorescens, Pseudomonas fulva, Pseudomonas lutea, Pseudomonas mucidolens, Pseudomonas oleovorans, Pseudomonas orientalis, Pseudomonas pseudoalcaligenes, Pseudomonas psychrophila, Pseudomonas putida, Pseudomonas synxantha, Pseudomonas syringae, Pseudomonas tolaasii, Pseudopropionibacterium propionicum, Rahnella aquatilis, Ralstonia pickettii, Ralstonia solanacearum, Raoultella planticola, Rhizobacter dauci, Rhizobium etli, Rhodococcus fascians, Rhodopseudomonas palustris, Roseburia intestinalis, Roseburia inulinivorans, Rothia mucilaginosa, Ruminococcus bromii, Ruminococcus gnavus, Ruminococcus torques, Sanguibacter keddieii, Sediminibacterium salmoneum, Selenomonas bovis, Serratia fonticola, Serratia liquefaciens, Serratia marcescens, Shewanella algae, Shewanella amazonensis, Shigella boydii, Shigella sonnei, Slackia exigua, Sneathia amnii, Sneathia sanguinegens, Solobacterium moorei, Sorangium cellulosum, Sphingobium amiense, Sphingobium japonicum, Sphingobium yanoikuyae, Sphingomonas wittichii, Sporosarcina aquimarina, Staphylococcus aureus, Staphylococcus auricularis, Staphylococcus capitis, Staphylococcus epidermidis, Staphylococcus haemolyticus, Staphylococcus hominis, Staphylococcus lugdunensis, Staphylococcus saprophyticus, Staphylococcus schleiferi, Staphylococcus simiae, Staphylococcus simulans, Staphylococcus warneri, Stenotrophomonas maltophilia, Stenoxybacter acetivorans, Streptococcus agalactiae, Streptococcus anginosus, Streptococcus australis, Streptococcus equinus, Streptococcus gallolyticus, Streptococcus infantis, Streptococcus intermedius, Streptococcus lutetiensis, Streptococcus marimammalium, Streptococcus mitis, Streptococcus mutans, Streptococcus oralis, Streptococcus parasanguinis, Streptococcus phocae, Streptococcus pseudopneumoniae, Streptococcus salivarius, Streptococcus sanguinis, Streptococcus thermophilus, Sutterella wadsworthensis, Tannerella forsythia, Terrahaemophilus aromaticivorans, Treponema denticola, Treponema maltophilum, Treponema parvum, Treponema vincentii, Trueperella bernardiae, Turicella otitidis, Ureaplasma parvum, Ureaplasma urealyticum, Varibaculum cambriense, Variovorax paradoxus, Veillonella atypica, Veillonella dispar, Veillonella montpellierensis, Veillonella parvula, Virgibacillus proomii, Viridibacillus arenosi, Viridibacillus arvi, Weissella cibaria, Weissella soli, Xanthomonas campestris, Xanthomonas vesicatoria, Zobellia laminariae and Zoogloea ramigera.

In one embodiment, the targeted receiver bacteria are Escherichia coli.

In one embodiment, the targeted receiver bacteria are Klebsiella pneumoniae.

In one embodiment, the targeted receiver bacteria are Bacteroides thetaiotaomicron and/or Bacteroides faecis.

In one embodiment, the targeted receiver bacteria are Roseburia intestinalis.

In one embodiment, the targeted bacteria are Cutibacterium acnes more specifically the acne related Cutibacterium acnes from the phylogroup IA1 or RT4, RT5, RT8, RT9, RT10 or Clonal Complex(CC) CC1, CC3, CC4, more specifically the ST1, ST3, ST4.

In one embodiment, the targeted receiver bacteria are pathogenic bacteria. The targeted receiver bacteria can be virulent bacteria.

In a particular embodiment, the targeted receiver bacteria are involved in infections in the host. In a particular embodiment, the targeted receiver bacteria are associated with the triggering, progression, or aggravation of auto-immune diseases in the host. In a particular embodiment, the targeted receiver bacteria are associated with the triggering, progression or aggravation of tumors or metastasis in the host. In a particular embodiment, the targeted receiver bacteria are associated with the triggering, progression or aggravation of neurodegenerative disease in the host. In a particular embodiment, the targeted receiver bacteria are associated with the triggering, progression or aggravation of CNS related disease in the host. In a particular embodiment, the targeted receiver bacteria are associated with the resistance of the host towards treatments against infection, tumor, neurodegenerative disease, CNS related disease, autoimmune disease, and/or cancer.

The targeted receiver bacteria can be antibacterial resistant bacteria, including those selected from the group consisting of extended-spectrum beta-lactamase-producing (ESBL) Escherichia coli, ESBL Klebsiella pneumoniae, vancomycin-resistant Enterococcus (VRE), methicillin-resistant Staphylococcus aureus (MRSA), multidrug-resistant (MDR) Acinetobacter baumannii, MDR Enterobacter spp., and a combination thereof. The targeted receiver bacteria can be selected from the group consisting of extended-spectrum beta-lactamase-producing (ESBL) Escherichia coli strains. In a particular embodiment, said targeted receiver bacteria are ESBL Escherichia coli and/or ESBL Klebsiella pneumoniae.

Alternatively, the targeted receiver bacterium can be a bacterium of the microbiome of a given species, in particular a bacterium of the human microbiota.

### Given Effect and Corresponding Nucleic Acids of Interest

In the modulating method of the invention, said nucleic acid of interest produces a given effect on said targeted receiver bacterial cell, as defined above.

By “nucleic acid producing a given effect on said targeted receiver bacterial cell” is meant herein that the delivery of said nucleic acid into said targeted receiver bacterial cell induces, directly or indirectly, a reaction into said targeted receiver bacterial cell (such as the expression of a RNA, the expression of a protein or the activation or the inhibition of an activity), wherein said reaction in said targeted receiver bacterial cell, preferably further generates, directly or indirectly, a reaction in said organism hosting said targeted receiver bacterial cell.

In a particular embodiment, the nucleic acid of interest is expressed in said targeted receiver bacterial cell, thereby producing said given effect. Expression of said nucleic acid of interest includes expression into a coding or non-coding RNA, or expression into a protein. Alternatively, in a particular embodiment, the nucleic acid of interest is not expressed in said targeted receiver bacterial cell, and the presence of said nucleic acid of interest in said targeted receiver bacterial cell produces said given effect (for example by providing binding regions to molecules already present in said targeted receiver bacterial cell).

In the context of the invention, said given effect may be selected from the group consisting of killing the receiver bacterial cell, making the receiver bacterial cell stop producing a given molecule, making the receiver bacterial cells reducing its level of production of a given molecule, and making the receiver bacterial cell produce a molecule of interest.

### Making the Receiver Bacterial Cell Produce a Molecule of Interest

In a particular embodiment, said given effect is making the receiver bacterial cell produce a molecule of interest, in particular a host modulatory molecule.

In another particular embodiment, said given effect is making the receiver bacterial cell produce, as molecule of interest, transcription factors and/or modified nucleases, in particular to activate specific pathways or genes in the bacteria that are naturally turned off.

In another particular embodiment, said given effect is making the receiver bacterial cell produce a molecule of interest which increases or decreases, preferably temporarily, the fitness of said receiver bacterial cell to its environment, in particular compared to other members of the microbiome which are not receiver bacterial cells.

In another particular embodiment, said given effect is making the receiver bacterial cell produce, as molecule of interest, a molecule of interest which acts on the microbiome environment, in particular without generating an effect at the level of the host organism cells.

By “host modulatory molecule” or “HMM” is meant herein any molecule, produced by said receiver bacterial cell, that acts, directly or indirectly, at the level of the host organism.

Said HMM may be of any nature. In particular, said HMM may be selected from the group consisting of non-coding nucleic acids, coding nucleic acids, proteins, lipids, sugars, LPS, metabolites and small molecules.

Examples of non-coding nucleic acids typically include non-coding DNAs or non-coding RNAs, such as siRNAs.

Examples of coding nucleic acids typically include coding DNAs or coding RNAs.

Examples of proteins typically include cytokines, such as chemokines, interferons, interleukins, lymphokines, tumour necrosis factors and anti-inflammatory cytokines; surface layer proteins, such as SIpB, in particular from Propionibacterium freudenreichii; microbial anti-Inflammatory molecule (MAM), such as MAM from Faecalibacterium prausnitz; antibodies such as monoclonal antibodies, multispecific antibodies, chimeric antibodies, antibody fragments and derivatives thereof; nanobodies; enzymes, in particular enzymes leading to the production of other HMMs; peptides such as Immune Selective Anti-Inflammatory Derivatives (FEG, Salivary gland derived peptides), and mimic proteins or peptides derived from the microbiome that mimic antigens from cells of the subject.

Mimic peptides of particular interest are bacterial mimic peptides that are associated with auto-immune diseases, for example those mentioned in Negi et al. (2017) Plos One 12:e0180518, which are hereby incorporated by reference. Of particular interest are the gene sequences encoding any of the mimic peptides in S1 Table of Negi et al.

Examples of lipids typically include SCFAs, such as butyrate.

Examples of small molecules typically include cyclosporin, nonsteroidal anti-inflammatory drugs (NSAIDs), steroidal anti-inflammatory drugs (SAIDs) and ROS.

Said HMM may further have any effect. In a particular embodiment, said HMM may be a molecule that will affect the immune system of the host, the host CNS and/or the host metabolism.

In particular, said HMM may be selected from the group consisting of anticancer molecules, antibiotic molecules, anti-viral molecules, anti-parasite molecules, anti-protozoal molecules, anesthetic molecules, anticoagulant molecules, inhibitors of an enzyme, steroidal molecules, anti-inflammatory molecules, antihistamine molecules, immunosuppressant molecules, anti-neoplastic molecules, antigens, vaccines, antibodies, decongestant molecules, sedative molecules, analgesic molecules, antipyretic molecules, hormones, anti-hormone molecules, anticholinergic agents, antidepressant molecules, antipsychotic molecules, neurotoxin molecules, hypnotic molecules, tranquilizer molecules, anticonvulsant molecules, muscle relaxant molecules, anti-aging molecules, anti-neurodegeneration molecules, neuromodulators, antispasmodic molecules, muscle contractant molecules, channel blocker molecules, miotic molecules, anti-secretory molecules, anti-thrombotic molecules, diuretic molecules, cardiovascular active molecules, vasoactive molecules, vasodilating molecules, anti-hypertensive molecules, angiogenic molecules, modulators of cell-extracellular matrix interactions (e.g. cell growth inhibitors and anti-adhesion molecules), growth factors, differentiation factors, antioxidant molecules, inhibitors of DNA, RNA, or protein synthesis, apoptosis factors, anti-apoptosis molecules, or anti-UV molecules.

Said HMM may further be of any origin. In particular, said HMM may be selected from the group consisting of host endogenous molecules, host exogenous molecules expressed naturally by other organisms, and synthetic compounds.

By “host endogenous molecule” is meant herein any molecule naturally produced by the host subject, in particular by a healthy host subject.

By “host exogenous molecule expressed naturally by other organisms” is meant herein any molecule which is not produced by the host subject (or by a subject of the same species as the host species) but which is naturally produced by another organism, in particular an organism from another species, from another gender, from another family, from another class or from another kingdom. Typically, said host exogenous molecule expressed naturally by other organisms may be a molecule produced by bacteria, in particular by microbiota.

In a particular embodiment, the nucleic acid of interest encodes a bacteriocin or a lysin, which can be a proteinaceous toxin produced by receiver bacteria to kill or inhibit growth of other bacteria. Bacteriocins are categorized in several ways, including producing strain, common resistance mechanisms, and mechanism of killing. Such bacteriocin had been described from gram negative bacteria (e.g. microcins, colicin-like bacteriocins and tailocins) and from gram positive bacteria (e.g. Class I, Class II, Class III or Class IV bacteriocins).

In one embodiment, the nucleic acid of interest encodes a toxin selected in the group consisting of microcins, colicin-like bacteriocins, tailocins, Class I, Class II, Class III and Class IV bacteriocins.

In a particular embodiment, the corresponding immunity polypeptide (i.e. anti-toxin) may be used to protect receiver bacterial cells (see review by Cotter et al., Nature Reviews Microbiology 11: 95, 2013).

By “synthetic compound” is meant herein any molecule which is neither naturally produced by the host subject (or by a subject of the same species as the host species) nor by another organism, in particular an organism from another species, from another gender, from another family, from another class or from another kingdom.

Said molecule of interest may further be produced by said targeted receiver bacterial cell in any form. In particular, said HMM may be selected from the group consisting of secreted molecules, intracellular molecules and membrane-displayed molecules.

The production of said molecule of interest by said targeted receiver bacterial cell may require the delivery of a nucleic acid of interest which includes one or more type(s) of gene(s) or group(s) of genes. In particular, said nucleic acid of interest may be selected from the group consisting of a gene encoding said molecule of interest, in particular said HMM, several genes encoding a protein complex that is the molecule of interest, in particular the HMM, a gene or group of genes encoding enzyme(s) of a metabolic pathway leading to the production of the molecule of interest, in particular of the HMM, a coding nucleic acid which is the molecule of interest, in particular the HMM, and a non-coding nucleic acid which is the molecule of interest, in particular the HMM.

### Making the Receiver Bacterial Cell Stop Producing a Given Molecule

In a particular embodiment, said given effect is making the receiver bacterial cell stop producing a given molecule.

By “making the receiver bacterial cell stop producing a given molecule” is meant herein reducing or abolishing the production of said given molecule by said bacterial cell and/or making the receiver bacterial cell produce a variant of said given molecule.

Typically, said given molecule the production of which is to be stopped has a negative effect on said host organism.

In a particular embodiment, said given molecule the production of which is to be stopped affects the fitness of said receiver bacterial cell to its environment. In a particular embodiment, making the receiver bacterial cell stop producing said given molecule, increases or decreases, preferably temporarily, the fitness of said receiver bacterial cell to its environment, in particular compared to other members of the microbiome which are not receiver bacterial cell.

In a particular embodiment, said given molecule may be selected from the group consisting of a toxin, a toxic factor, a virulence protein, a virulence factor, a protein encoded by an antibiotic resistance gene, a protein encoded by a remodeling gene or by a modulatory gene. In a particular embodiment, said given effect is to selectively remove antibiotic resistance from antibiotic resistant bacterial strains.

In a particular embodiment, said nucleic acid of interest is a gene or group of genes encoding one or more exogenous enzyme(s) which result(s) in a genetic modification.

In a particular embodiment, said nucleic acid of interest is a gene encoding a base-editor or a prime-editor.

In some embodiments, the genetic modification is made with one or more of the following enzymes and systems.

Cytosine base editors (CBE) and Adenosine base editors (ABE), as described in Rees et al. (2018) Nat Rev Genet 19:770-788, which is hereby incorporated by reference.

So far there are seven types of DNA base editors described:
Cytosine Base Editor (CBE) that convert C:G into T:A (Komor et al. (2016) Nature 533:420-424)
Adenine Base Editor (ABE) that convert A:T into G:C (Gaudelli et al. (2017) Nature 551:464-471)
Cytosine Guanine Base Editor (CGBE) that convert C:G into G:C (Chen et al. (2020) Biorxiv “Precise and programmable C:G to G:C base editing in genomic DNA”; Kurt et al. (2020) Nat. Biotechnol. “CRISPR C-to-G base editors for inducing targeted DNA transversions in human cells”)
Cytosine Adenine Base Editor (CABE) that convert C:G into A:T (Zhao et al. (2020) Nature Biotechnol. “New base editors change C to A in bacteria and C to G in mammalian cells”)
Adenine Cytosine Base Editor (ACBE) that convert A:T into C:G (WO2020181180)
Adenine Thymine Base Editor (ATBE) that convert A:T into T:A (WO2020181202)
Thymine Adenine Base Editor (TABE) that convert T:A into A:T (WO2020181193, WO2020181178, WO2020181195)

Base editors differ in the base modification enzymes. CBE rely on ssDNA cytidine deaminase among which: APOBEC1, rAPOBEC1, APOBEC1 mutant or evolved version (evoAPOBEC1), and APOBEC homologs (APOBEC3A (eA3A), Anc689), Cytidine deaminase 1 (CDA1), evoCDA1, FERNY, evoFERNY.

ABE rely on deoxyadenosine deaminase activity of a tandem fusion TadA-TadA* where TadA* is an evolved version of TadA, an E. coli tRNA adenosine deaminase enzyme, able to convert adenosine into Inosine on ssDNA.TadA* include TadA-8a-e and TadA-7.10.

Except from base modification enzyme there has been also modifications implemented to base editor to increase editing efficacy, precision and modularity:
the addition of one or two uracil DNA glycosylase inhibitor domain (UGI) to prevent base excision repair mechanism to revert base edition
the addition of Mu-GAM that decrease insertion-deletion rate by inhibiting Non-homologous end joining mechanism in the cell (NHEJ)
the use of nickase active Cas9 (nCas9 D10A) that, by creating nicks on the non-edited strand favor its repair and consequently the fixation of the edited base
the use of diverse Cas proteins from for example different organisms, mutants with different PAM motifs or different fidelity or different family (e.g. Cas12a).

Non-limiting examples of DNA based editor proteins include BE1, BE2, BE3, BE4, BE4-GAM, HF-BE3, Sniper-BE3, Target-AID, Target-AID-NG, ABE, EE-BE3, YE1-BE3, YE2-BE3, YEE-BE3, BE-PLUS, SaBE3, SaBE4, SaBE4-GAM, Sa(KKH)-BE3, VQR-BE3, VRER-BE3, EQR-BE3, xBE3, Cas12a-BE, Ea3A-BE3, A3A-BE3, TAM, CRISPR-X, ABE7.9, ABE7.10, ABE7.10*, xABE, ABESa, VQR-ABE, VRER-ABE, Sa(KKH)-ABE, ABE8e, SpRY-ABE, SpRY-CBE, SpG-CBE4, SpG-ABE, SpRY-CBE4, SpCas9-NG-ABE, SpCas9-NG-CBE4, enAsBE1.1, enAsBE1.2, enAsBE1.3, enAsBE1.4, AsBE1.1, AsBE1.4, CRISPR-Abest, CRISPR-Cbest, eA3A-BE3, AncBE4.

Cytosine Guanine Base Editors (CGBE) consist of a nickase CRISPR fused to:
a. A cytosine deaminase (rAPOBEC) and base excision repair proteins (e.g. rXRCC1) (Chen et al. (2020) Biorxiv “Precise and programmable C:G to G:C base editing in genomic DNA”).
b. A rat APOBEC1 variant (R33A) protein and an E. coli-derived uracil DNA N-glycosylase (eUNG) (Kurt et al. (2020) Nat. Biotechnol. “CRISPR C-to-G base editors for inducing targeted DNA transversions in human cells”).

Cytosine Adenine Base Editors (CABE) consist of a Cas9 nickase, a cytidine deaminase (e.g. AID), and a uracil-DNA glycosylase (Ung) (Zhao et al. (2020) Nature Biotechnol. “New base editors change C to A in bacteria and C to G in mammalian cells”).

ACBE include a nucleic acid programmable DNA-binding protein and an adenine oxidase (WO2020181180).

ATBE consist of a Cas9 nickase and one or more adenosine deaminase or an oxidase domain (WO2020181202).

TABE consist of a Cas9 nickase and an adenosine methyltransferase, a thymine alkyltransferase, or an adenosine deaminase domain (WO2020181193, WO2020181178, WO2020181195).

Base editor molecules can also consist of two or more of the above listed editor enzymes fused to a Cas protein (e.g. combination of an ABE and CBE). These biomolecules are named dual base editors and enable the editing of two different bases (Grunewald et al. (2020) Nature Biotechnol. “A dual-deaminase CRISPR base editor enables concurrent adenine and cytosine editing”; Li et al. (2020) Nature Biotechnol. “Targeted, random mutagenesis of plant genes with dual cytosine and adenine base editors”).

Prime editors (PE), as described in Anzalone et al. (2019) Nature 576:149-157, which is hereby incorporated by reference, consist of nCas9 fused to a reverse transcriptase used in combination with a prime editing RNA (pegRNA, a guide RNA that includes a template region for reverse transcription).

Prime Editing allows introduction of insertions, deletions (indels) and 12 base-to-base conversions. Prime editing relies on the ability of a reverse transcriptase (RT), fused to a Cas nickase variant, to convert RNA sequence brought by a prime editing guide RNA (pegRNA) into DNA at the nick site generated by the Cas protein. The DNA flap generated from this process is then included or not in the targeted DNA sequence.

Prime editing systems include:
a Cas nickase variant such as Cas9-H840A fused to a reverse transcriptase domain such as M-MLV RT or its mutant version (M-MLV RT(D200N), M-MLV RT(D200N/L603W), M-MLV RT(D200N/L603W/T330P/T306K/W313F)
a prime editing guide RNA (pegRNA)

To favor editing the prime editing system can include the expression of an additional sgRNA targeting the Cas nickase activity towards the non-edited DNA strand ideally only after the resolution of the edited strand flap by designing the sgRNA to anneal with the edited strand but not with the original strand.

Non-limiting examples of prime editing systems include PE1, PE1-M1, PE1-M2, PE1-M3, PE1-M6, PE1-M15, PE1-M3inv, PE2, PE3, PE3b.

Cas9 Retron precISe Parallel Editing via homologY (‘CRISPEY’), a retron RNA fused to the sgRNA and expressed together with Cas9 and the retron proteins including at least the reverse transcriptase (Sharon et al. (2018) Cell 175:544-557.e16).

The SCRIBE strategy: a retron system expressed in combination with a recombinase promoting the recombination of single stranded DNA, also known as single stranded annealing proteins (SSAPs) (Farzadfard & Lu (2014) Science 346:1256272). Such recombinases include but are not limited to phage recombinases such as lambda red, recET, Sak, Sak4, and newly described SSAPs described in Wannier et al. (2020) Proc Natl Acad Sci USA 117(24):13689-13698 which is hereby incorporated by reference.

The targetron system based on group II introns described in Karberg et al. (2001) Nat Biotechnol 19:1162-7, which is hereby incorporated by reference, and which has been adapted to many bacterial species.

Other retron based gene targeting approaches are described in Simon et al. (2019) Nucleic Acids Res 47:11007-11019, which is hereby incorporated by reference.

In a particular embodiment, the CRISPR system is included in the nucleic acid of interest. The CRISPR system contains two distinct elements, i.e. i) an endonuclease, in this case the CRISPR associated nuclease (Cas or “CRISPR associated protein”) and ii) a guide RNA. The guide RNA may be in the form of a chimeric RNA which consists of the combination of a CRISPR (RNAcr) bacterial RNA and a RNAtracr (trans-activating RNA CRISPR) (Jinek et al. (2012) Science 337(6096):816-21). The guide RNA combines the targeting specificity of the RNAcr corresponding to the “spacing sequences” that serve as guides to the Cas proteins, and the conformational properties of the RNAtracr in a single transcript. When the guide RNA and the Cas protein are expressed simultaneously in the cell, the target genomic sequence can be permanently modified or interrupted. The modification is advantageously guided by a repair matrix. In general, the CRISPR system includes two main classes depending on the nuclease mechanism of action. Class 1 is made of multi-subunit effector complexes and includes type I, III and IV. Class 2 is made of single-unit effector modules, like Cas9 nuclease, and includes type II (II-A,II-B,II-C,II-C variant), V (V-A,V-B,V-C,V-D,V-E,V-U1,V-U2,V-U3,V-U4,V-U5) and VI (VI-A,VI-B1,VI-B2,VI-C,VI-D)

The nucleic acid of interest according to the present disclosure may comprise a nucleic acid sequence encoding Cas protein. A variety of CRISPR enzymes are available for use as a sequence of interest on the plasmid. In some embodiments, the CRISPR enzyme is a Type II CRISPR enzyme. In some embodiments, the CRISPR enzyme catalyzes DNA cleavage. In some other embodiments, the CRISPR enzyme catalyzes RNA cleavage. Preferably, the CRISPR enzyme does not make a double strand break. In some embodiments, the CRISPR enzyme makes a single strand break or nicks. In some embodiments, the CRISPR enzyme does not make any break in the DNA or RNA. In one embodiment, a Cas13-deaminase fusion is used to base edit an RNA.

In one embodiment, the CRISPR enzymes may be coupled to a sgRNA. In certain embodiments, the sgRNA targets a gene encoding a given molecule as defined above.

Non-limiting examples of Cas proteins as part of a multi-subunit effector or as a single-unit effector include Cas1, Cas1B, Cas2, Cas3, Cas4, Cas5, Cas6, Cas7, Cas8, Cas9 (also known as Csn1 and Csx12), Cas10, Cas11 (SS), Cas12a (Cpf1), Cas12b (C2c1), Cas12c (C2c3), Cas12d (CasY), Cas12e (CasX), C2c4, C2c8, C2c5, C2c10, C2c9, Cas13a (C2c2), Cas13b (C2c6), Cas13c (C2c7), Cas13d, Csa5, Csc1, Csc2, Cse1, Cse2, Csy1, Csy2, Csy3, Csf1, Csf2, Csf3, Csf4, Csm2, Csm3, Csm4, Csm5, Csm6, Cmr1, Cmr3, Cmr4, Cmr5, Cmr6, Csn2, Csb1, Csb2, Csb3, Csx17, Csx14, Csx10, Csx16, CsaX, Csx13, Csx1, Csx15, SdCpf1, CmtCpf1, TsCpf1, CmaCpf1, PcCpf1, ErCpf1, FbCpf1, UbcCpf1, AsCpf1, LbCpf1, Mad4, Mad7, Cms1, homologues thereof, orthologues thereof, variants thereof, or modified versions thereof. In some embodiments, the CRISPR enzyme cleaves both strands of the target nucleic acid at the Protospacer Adjacent Motif (PAM) site.

In various embodiments, the invention encompasses fusion proteins comprising a Cas9 (e.g., a Cas9 nickase) domain and a deaminase domain. In some embodiments, the fusion protein comprises Cas9 and a cytosine deaminase enzyme, such as APOBEC enzymes, or adenosine deaminase enzymes, such as ADAT enzymes, for example as disclosed in U.S. Patent Publ. 2015/0166980, which is hereby incorporated by reference. In one embodiment, the deaminase is an ACF1/ASE deaminase.

In various embodiments, the APOBEC deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In various embodiments, the fusion protein comprises a Cas9 domain, a cytosine deaminase domain, and a uracil glycosylase inhibitor (UGI) domain.

In one embodiment, the deaminase is an adenosine deaminase that deaminate adenosine in DNA, for example as disclosed in U.S. Pat. No. 10,113,163, which is hereby incorporated by reference. In some embodiments, the fusion proteins further comprise an inhibitor of base repair, such as, a nuclease dead inosine specific nuclease (dISN), for example as disclosed in U.S. Pat. No. 10,113,163. In various embodiments, the invention encompasses fusion proteins comprising a catalytically impaired Cas9 endonuclease fused to an engineered reverse transcriptase, programmed with a prime editing guide RNA (pegRNA) that both specifies the target site and encodes the desired edit, for example as described in Anzalone et al. (2019) Nature 576:149-157, which is hereby incorporated by reference.

In a particular embodiment, the CRISPR enzyme is any Cas protein, in particular any Cas9 protein, for instance any naturally occurring bacterial Cas9 as well as any variants, chimeras, homologs or orthologs thereof.

By “Cas9” is meant a protein Cas9 (also called Csn1 or Csx12) or a functional protein, peptide or polypeptide fragment thereof, i.e. capable of interacting with the guide RNA(s) and of exerting the enzymatic activity (nuclease) which allows it to perform the double-strand cleavage of the DNA of the target genome. “Cas9” can thus denote a modified protein, for example truncated to remove domains of the protein that are not essential for the predefined functions of the protein, in particular the domains that are not necessary for interaction with the gRNA(s).

The sequence encoding Cas9 (the entire protein or a fragment thereof) as used in the context of the disclosure can be obtained from any known Cas9 protein (Fonfara et al. (2014) Nucleic Acids Res 42(4):2577-90; Shmakov et al. (2017) Nat Rev Microbiol 15(3):169-182). Examples of Cas9 proteins useful in the present disclosure include, but are not limited to, Cas9 proteins of Streptococcus pyogenes (SpCas9), Streptococcus thermophiles (St1Cas9, St3Cas9), Streptococcus mutans, Staphylococcus aureus (SpCas9), Campylobacter jejuni (CjCas9), Francisella novicida (FnCas9) and Neisseria meningitides (NmCas9).

The sequence encoding Cpf1 (Cas12a) (the entire protein or a fragment thereof) as used in the context of the disclosure can be obtained from any known Cpf1 (Cas12a) protein (Koonin et al. (2017) Current Opinion in Microbiology 37:67-78). Examples of Cpf1(Cas12a) proteins useful in the present disclosure include, but are not limited to, Cpf1(Cas12a) proteins of Acidaminococcus sp, Lachnospiraceae bacteriu and Francisella novicida.

The sequence encoding Cas13a (the entire protein or a fragment thereof) can be obtained from any known Cas13a (C2c2) protein (Abudayyeh et al. (2017) Nature 550:280). Examples of Cas13a (C2c2) proteins useful in the present disclosure include, but are not limited to, Cas13a (C2c2) proteins of Leptotrichia wadei (LwaCas13a).

The sequence encoding Cas13d (the entire protein or a fragment thereof) can be obtained from any known Cas13d protein (Yan et al. (2018) Mol Cell 70(2):327-339). Examples of Cas13d proteins useful in the present disclosure include, but are not limited to, Cas13d proteins of Eubacterium siraeum and Ruminococcus sp.

The sequence encoding Mad4 (the entire protein or a fragment thereof) as used in the context of the invention is disclosed in international application WO2018/236548.

The sequence encoding Mad7 (the entire protein or a fragment thereof) as used in the context of the invention is disclosed in international application WO2018/236548.

The sequence encoding Cms1 (the entire protein or a fragment thereof) as used in the context of the invention is disclosed in international patent application WO2017/141173.

In some embodiments, other programmable nucleases can be used. These include an engineered TALEN (Transcription Activator-Like Effector Nuclease) and variants, engineered zinc finger nuclease (ZFN) variants, natural, evolved or engineered meganuclease or recombinase variants, and any combination or hybrids of programmable nucleases. Thus, the programmable nucleases provided herein may be used to selectively modify DNA encoding a gene of interest such as, for example, a toxin gene, a virulence factor gene, an antibiotic resistance gene, a remodeling gene or a modulatory gene (cf. WO2014124226 and US2015/0064138).

In some embodiments, the genetic modification is made at the RNA level. RNA base editing is based on the same principle as DNA base editing: an enzyme catalyzing the conversion of a RNA base into another must be brought close to the target base to perform its conversion locally. In one embodiment, the enzyme used for RNA editing is an adenosine deaminase from ADAR family that converts Adenosine into Inosine in dsRNA structure. Several seminal studies used this specificity for dsRNA and fused the ADAR deaminase domain (ADARDD) to an antisense oligo in order to program local RNA base editing. More recently the ability of some CRISPR-Cas systems to bind RNA molecules was repurposed into RNA editing. Using catalytically dead Cas13b enzyme (dPspCas13b) fused to a hyperactive mutant of ADAR2 deaminase domain (ADAR2DD-E488Q for REPAIRv1 and ADAR2DD-E488Q-T375G for REPAIRv2) Cox et al improved specificity and efficiency compare to previous RNA editing strategies. Non-limiting examples of RNA based editor proteins include REPAIRv1, REPAIRv2

In a particular embodiment, the modification is made in a gene selected in the group consisting of an antibiotic resistance gene, virulence factor or protein gene, toxin factor or protein gene, a gene expressing a bacterial receptor, a membrane protein, a structural protein, a secreted protein, and a gene expressing resistance to a drug in general.

In one embodiment, the modification is made to target and inactivate a virulence factor. A virulence factor can be any substance produced by a pathogen that alters host-pathogen interaction by increasing the degree of damage done to the host. Virulence factors are used by pathogens in many ways, including, for example, in cell adhesion or colonization of a niche in the host, to evade the host's immune response, to facilitate entry to and egress from host cells, to obtain nutrition from the host, or to inhibit other physiological processes in the host. Virulence factors can include enzymes, endotoxins, adhesion factors, motility factors, factors involved in complement evasion, and factors that promote biofilm formation. For example, such targeted virulence factor gene can be E. coli virulence factor gene such as, without limitation, EHEC-HlyA, Stx1 (VT1), Stx2 (VT2), Stx2a (VT2a), Stx2b (VT2b), Stx2c (VT2c), Stx2d (VT2d), Stx2e (VT2e) and Stx2f (VT2f), Stx2h (VT2h), fimA, fimF, fimH, neuC, kpsE, sfa, foc, iroN, aer, iha, papC, papGI, papGII, papGIII, hlyC, cnf1, hra, sat, ireA, usp ompT, ibeA, malX, fyuA, irp2, traT, afaD, ipaH, eltB, estA, bfpA, eaeA, espA, aaiC, aatA, TEM, CTX, SHV, csgA, csgB, csgC, csgD, csgE, csgF, csgG, csgH, T1SS, T2SS, T3SS, T4SS, T5SS, T6SS (secretion systems). For example, such targeted virulence factor gene can be Shigella dysenteriae virulence factor gene such as, without limitation, stx1 and stx2. For example, such targeted virulence factor gene can be Yersinia pestis virulence factor gene such as, without limitation, yscF (plasmid-borne (pCDI) T3SS external needle subunit). For example, such targeted virulence factor gene can be Francisella tularensis virulence factor gene such as, without limitation, fslA. For example, such targeted virulence factor gene can be Bacillus anthracis virulence factor gene such as, without limitation, pag (Anthrax toxin, cell-binding protective antigen). For example, such targeted virulence factor gene can be Vibrio cholera virulence factor gene such as, without limitation, ctxA and ctxB (cholera toxin), tcpA (toxin co-regulated pilus), and toxT (master virulence regulator). For example, such targeted virulence factor gene can be Pseudomonas aeruginosa virulence factor genes such as, without limitation, pyoverdine (e.g., sigma factor pvdS, biosynthetic genes pvdL, pvdI, pvdJ, pvdH, pvdA, pvdF, pvdQ, pvdN, pvdM, pvdO, pvdP, transporter genes pvdE, pvdR, pvdT, opmQ), siderophore pyochelin (e.g., pchD, pchC, pchB, pchA, pchE, pchF and pchG, and toxins (e.g., exoU, exoS and exoT). For example, such targeted virulence factor gene can be Klebsiella pneumoniae virulence factor genes such as, without limitation, fimA (adherence, type I fimbriae major subunit), and cps (capsular polysaccharide). For example, such targeted virulence factor gene can be Acinetobacter baumannii virulence factor genes such as, without limitation, ptk (capsule polymerization) and epsA (assembly). For example, such targeted virulence factor gene can be Salmonella enterica Typhi virulence factor genes such as, without limitation, MIA (invasion, SPI-1 regulator), ssrB (SPI-2 regulator), and those associated with bile tolerance, including efflux pump genes acrA, acrB and tolC. For example, such targeted virulence factor gene can be Fusobacterium nucleatum virulence factor genes such as, without limitation, FadA and TIGIT. For example, such targeted virulence factor gene can be Bacteroides fragilis virulence factor genes such as, without limitation, bft.

In another embodiment, the modification is made in an antibiotic resistance gene such as, without limitation, GyrB, ParE, ParY, AAC(1), AAC(2′), AAC(3), AAC(6′), ANT(2″), ANT(3″), ANT(4′), ANT(6), ANT(9), APH(2″), APH(3″), APH(3′), APH(4), APH(6), APH(7″), APH(9), ArmA, RmtA, RmtB, RmtC, Sgm, AER, BLA1, CTX-M, KPC, SHV, TEM, BlaB, CcrA, IMP, NDM, VIM, ACT, AmpC, CMY, LAT, PDC, OXA p-lactamase, mecA, Omp36, OmpF, PIB, bla (blaI, blaR1) and mec (mecI, mecR1) operons, Chloramphenicol acetyltransferase (CAT), Chloramphenicol phosphotransferase, Ethambutol-resistant arabinosyltransferase (EmbB), MupA, MupB, Integral membrane protein MprF, Cfr 23S rRNA methyltransferase, Rifampin ADP-ribosyltransferase (Arr), Rifampin glycosyltransferase, Rifampin monooxygenase, Rifampin phosphotransferase, DnaA, RbpA, Rifampin-resistant beta-subunit of RNA polymerase (RpoB), Erm 23S rRNA methyltransferases, Lsa, MsrA, Vga, VgaB, Streptogramin Vgb lyase, Vat acetyltransferase, Fluoroquinolone acetyltransferase, Fluoroquinolone-resistant DNA topoisomerases, Fluoroquinolone-resistant GyrA, GyrB, ParC, Quinolone resistance protein (Qnr), FomA, FomB, FosC, FosA, FosB, FosX, VanA, VanB, VanD, VanR, VanS, Lincosamide nucleotidyltransferase (Lin), EreA, EreB, GimA, Mgt, Ole, Macrolide phosphotransferases (MPH), MefA, MefE, Mel, Streptothricin acetyltransferase (sat), Sul1, Sul2, Sul3, sulfonamide-resistant FolP, Tetracycline inactivation enzyme TetX, TetA, TetB, TetC, Tet30, Tet31, TetM, TetO, TetQ, Tet32, Tet36, MacAB-ToIC, MsbA, MsrA,VgaB, EmrD, EmrAB-ToIC, NorB, GepA, MepA, AdeABC, AcrD, MexAB-OprM, mtrCDE, EmrE, adeR, acrR, baeSR, mexR, phoPQ, mtrR, or any antibiotic resistance gene described in the Comprehensive Antibiotic Resistance Database (CARD https://card.mcmaster.ca/).

In preferred embodiments, the antibiotic is selected from the group consisting of penicillins such as penicillin G, penicillin K, penicillin N, penicillin O, penicillin V, methicillin, benzylpenicillin, nafcillin, oxacillin, cloxacillin, dicloxacillin, ampicillin, amoxicillin, pivampicillin, hetacillin, bacampicillin, metampicillin, talampicillin, epicillin, carbenicillin, ticarcillin, temocillin, mezlocillin, and piperacillin; cephalosporins such as cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, loracarbef, cefbuperazone, cefminox, cefotetan, cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, latamoxef, cefclidine, cefepime, cefluprenam, cefoselis, cefozopran, cefpirome, cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; polymyxins such as polysporin, neosporin, polymyxin B, and polymyxin E, rifampicins such as rifampicin, rifapentine, and rifaximin; Fidaxomicin; quinolones such as cinoxacin, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, temafloxacin, tosufloxacin, clinafloxacin, gatifloxacin, gemifloxacin, moxifloxacin, sitafloxacin, trovafloxacin, prulifloxacin, delafloxacin, nemonoxacin, and zabofloxacin; sulfonamides such as sulfafurazole, sulfacetamide, sulfadiazine, sulfadimidine, sulfafurazole, sulfisomidine, sulfadoxine, sulfamethoxazole, sulfamoxole, sulfanitran, sulfadimethoxine, sulfametho-xypyridazine, sulfametoxydiazine, sulfadoxine, sulfametopyrazine, and terephtyl; macrolides such as azithromycin, clarithromycin, erythromycin, fidaxomicin, telithromycin, carbomycin A, josamycin, kitasamycin, midecamycin, oleandomycin, solithromycin, spiramycin, troleandomycin, tylosin, and roxithromycin; ketolides such as telithromycin, and cethromycin; fluoroketolides such as solithromycin; lincosamides such as lincomycin, clindamycin, and pirlimycin; tetracyclines such as demeclocycline, doxycycline, minocycline, oxytetracycline, and tetracycline; aminoglycosides such as amikacin, dibekacin, gentamicin, kanamycin, neomycin, netilmicin, sisomicin, tobramycin, paromomycin, and streptomycin; ansamycins such as geldanamycin, herbimycin, and rifaximin; carbacephems such as loracarbef; carbapenems such as ertapenem, doripenem, imipenem (or cilastatin), and meropenem; glycopeptides such as teicoplanin, vancomycin, telavancin, dalbavancin, and oritavancin; lincosamides such as clindamycin and lincomycin; lipopeptides such as daptomycin; monobactams such as aztreonam; nitrofurans such as furazolidone, and nitrofurantoin; oxazolidinones such as linezolid, posizolid, radezolid, and torezolid; teixobactin, clofazimine, dapsone, capreomycin, cycloserine, ethambutol, ethionamide, isoniazid, pyrazinamide, rifabutin, arsphenamine, chloramphenicol, fosfomycin, fusidic acid, metronidazole, mupirocin, platensimycin, quinupristin (or dalfopristin), thiamphenicol, tigecycline, tinidazole, trimethoprim, alatrofloxacin, fidaxomycin, nalidixice acide, rifampin, derivatives and combination thereof.

When the antibiotic resistance gene is located in the bacterium on a plasmid without addiction systems, it is possible to eliminate the antibiotic resistance by cleavage either in the antibiotic resistance gene or anywhere else in the plasmid.

In another embodiment, the modification is made in a bacterial toxin gene. Bacterial toxins can be classified as either exotoxins or endotoxins. Exotoxins are generated and actively secreted; endotoxins remain part of the bacteria. The response to a bacterial toxin can involve severe inflammation and can lead to sepsis. Such toxin can be for example Botulinum neurotoxin, Tetanus toxin, Staphylococus toxins, Diphteria toxin, Anthrax toxin, Alpha toxin, Pertussis toxin, Shiga toxin, Heat-stable enterotoxin (E. coli ST), colibactin, BFT (B. fragilis toxin) or any toxin described in Henkel et al., (Toxins from Bacteria in EXS. 2010; 100: 1-29). In a particular embodiment, said toxin is Shiga toxin.

In some embodiments, the modification is made in a mimic peptide gene sequence so that the homology with the human peptide sequence is reduced, and therefore results in the mimic peptide being not recognized anymore by the host immune system. Mimic peptides of particular interest are bacterial mimic peptides that are associated with auto-immune diseases, for example those mentioned in Negi et al. (2017) Plos One 12:e0180518, which are hereby incorporated by reference. Of particular interest are the gene sequences encoding any of the mimic peptides in S1 Table of Negi et al.

In preferred embodiments, the mimic peptide is from Proteobacteria or Firmicutes. Of particular interest are the gene sequences encoding 24 gut bacterial peptides identified by Negi et al. with homology to four human peptides from Low molecular weight phosphotyrosine protein phosphatase, Aldehyde dehydrogenase family 3 member B1, Maleylacetoacetate isomerase and Uracil-DNA glycosylase. These gene sequences can be modified to reduce the homology with the human sequences and prevent cross-reactivity of those recognized by the host immune system with the human counterpart.

In a preferred embodiment, the genetic modification is in the Bacteroides faecis or Bacteroides thetaiotaomicron beta-galactosidase gene. Preferably, the Bacteroides faecis or Bacteroides thetaiotaomicron beta-galactosidase protein with the genetic modification shows lower homology with human MYH6 cardiac peptide as compared to the Bacteroides faecis or Bacteroides thetaiotaomicron beta-galactosidase protein without the genetic modification. Preferably the genetic modification is performed in the peptides fragment recognized as epitope by the human immune system leading to a weaker or absence of epitope recognition by the human immune system.

In a preferred embodiment, the genetic modification is in human commensal bacteria encoding a Ro60 ortholog gene. Preferably, the Ro60 protein resulting from the genetic modification shows lower homology with human Ro60 peptide as compared to the original protein. Preferably the genetic modification is performed in the DNA sequence corresponding to peptides fragment recognized as epitope by the human immune system leading to a weaker or absence of epitope recognition by the human immune system. Preferably the human bacterial commensal targeted for genetic modification are: Propionibacterium propionicum, Corynebacterium amycolatum, Actinomyces massiliensis, Bacteroides thetaiotaomicron. Even more preferably the human bacterial commensal targeted for genetic modification is Propionibacterium propionicum.

In a preferred embodiment, the genetic modification is in human commensal bacterial DNA sequence encoding a peptide that mimic insulin B 9-25, a self-epitope involved in type 1 diabetes. The genetic mutation reduces homology to the insulin B9-25 epitope SHLVEALYLVCGERGFF (SEQ ID NO: 1). In a preferred embodiment, the target bacteria belong to the Firmicutes phylum. In a preferred embodiment, the target gene in the target bacteria is part of the transketolase N superfamily.

In a preferred embodiment, the genetic modification is in Roseburia intestinalis encoding a peptide that mimic the epitope of the autoantigen β2-glycoprotein I (β2GPI), a self-epitope involved in antiphospholipid syndrome (APS). The genetic mutation is reducing homology to the T cell (β2GP1) epitope KVSFFCKNKEKKCSY (SEQ ID NO: 2) and/or B cell epitope VSRGGMRKFIC (SEQ ID NO: 3).

The genetic modification can be in the translated or untranslated regions of a gene. The genetic modification can be in the promoter region of a gene or within any other region involved in gene regulation. In some embodiments, the genetic modification results in the change in at least 1, 2, 3, 4, 5, 6, 7, 8, 9, 10, 11, 12, 13, 14, 15, 20, 25, 30, 35, 40, 45, 50, 100, 200, 500, etc. amino acids to a different amino acid. In some embodiments, the genetic modification introduces a stop codon. In some embodiments, the genetic modification is outside protein coding sequences, within RNA, or within regulatory sequences.

Preferably, the genetic modification does not integrate a phage genome or exogenous DNA into the host bacterial chromosome or endogenous plasmid(s). Preferably, the genetic modification does not result in expression of an exogenous protein from an integrated exogenous DNA in the host bacterial chromosome or endogenous plasmid(s). Most preferably, the genetic modification does not involve either NHEJ or HR endogenous repair mechanism of the host bacteria.

### Killing the Receiver Bacterial Cell

In a particular embodiment, said given effect is killing the receiver bacterial cell.

In a particular embodiment, said nucleic acid of interest is a gene encoding a nuclease.

In one embodiment, the nucleic acid of interest is a programmable nuclease circuit to be delivered to the targeted bacteria. This programmable nuclease circuit may be able to mediate in vivo sequence-specific elimination of bacteria that contain a target gene of interest (e.g. a gene that is harmful to humans). Some embodiments of the present disclosure relate to engineered variants of different CRISPR-Cas systems classes and types, such as the Type II CRISPR-Cas (Clustered Regularly Interspaced Short Palindromic Repeats-CRISPR-associated) system of Streptococcus pyogenes, as disclosed above. Other programmable nucleases that can be used include other CRISPR-Cas systems, engineered TALEN (Transcription Activator-Like Effector Nuclease) variants, engineered zinc finger nuclease (ZFN) variants, natural, evolved or engineered meganuclease or recombinase variants, and any combination or hybrids of programmable nucleases, as disclosed above. Thus, the programmable nuclease circuit provided herein may be used to selectively cleave DNA encoding a gene of interest such as, for example, a toxin gene, a virulence factor gene, an antibiotic resistance gene, a remodeling gene or a modulatory gene (cf. WO2014124226 and US2015/0064138).

In a particular embodiment, said nuclease is the Cpf1 nuclease.

In a particular embodiment, said nuclease is the Cas9 nuclease

In a particular embodiment, said nuclease is the Mad4 nuclease, as defined above.

In a particular embodiment, said nuclease is the Mad7 nuclease, as defined above.

In a particular embodiment, said nuclease is the Cms1 nuclease, as defined above.

In a particular embodiment, antibiotic resistant strains are targetly killed by programming the nuclease to perform a DNA cleavage, e.g. a double strand DNA break in an antibiotic resistance gene located on the chromosome of the target bacteria or on a plasmid with addictive systems (toxin/antitoxin).

Other sequences of interest, preferably programmable, can be delivered to targeted bacteria to kill it. For example, the nucleic acid of interest may encode holins or toxins.

In a particular embodiment, said nucleic acid of interest further makes the receiver bacterial cell produce a molecule of interest, as disclosed above, in particular a host modulatory molecule, as disclosed above, before being killed or just after being killed as a bacterial host for instance.

### Nucleic Acid of Interest

In the context of the invention, the nucleic acid of interest may be under the control of a promoter.

As known by the person skilled in the art, a promoter may be classified as strong or weak according to its affinity for RNA polymerase. The strength of a promoter may depend on whether initiation of transcription occurs at that promoter with high or low frequency. Different promoters with different strengths may be used in the present invention leading to different levels of gene/protein expression (e.g. the level of expression initiated from an mRNA originating from a weak promoter is lower than the level of expression initiated from a strong promoter).

It will be appreciated by those of ordinary skill in the art that a promoter sequence may be selected from a large number of known bacterial genes expressed by various bacterial species. Also, methods of prokaryotic promoter prediction exist, and can be based on DNA stability analysis as described in Kanhere and Bansal (BMC Bioinformatics 2005, 6:1). The choice of promoter on the vector according to the present invention can thus be made based on the bacteria to target.

In some embodiments, the nucleic acid of interest may be positioned under the control of a recombinant or heterologous promoter, which refers to a promoter that is not normally associated with the nucleic acid of interest in its natural environment.

Examples of bacterial promoters for use in accordance with the present invention include, without limitation, positively regulated E. coli promoters such as positively regulated a 70 promoters (e.g., inducible pBad/araC promoter, Lux cassette right promoter, modified lambda Prm promote, plac Or2-62 (positive), pBad/AraC with extra REN sites, pBad, P(Las) TetO, P(Las) CIO, P(Rhl), Pu, FecA, φRE, cadC, hns, pLas, pLux), a “s” promoter (e.g., Pdps), σ 32 promoters (e.g., heat shock) and σ 54 promoters (e.g., glnAp2); negatively regulated E. coli promoters such as negatively regulated σ 70 promoters (e.g., Promoter (PRM+), modified lambda Prm promoter, TetR-TetR-4C P(Las) TetO, P(Las) CIO, P(Lac) IQ, RecA_DlexO_DLac01, dapAp, FecA, Pspac-hy, pel, plux-cl, plux-lac, CinR, CinL, glucose controlled, modified Pr, modified Prm+, FecA, Pcya, rec A (SOS), Rec A (SOS), EmrR_regulated, BetI_regulated, pLac_lux, pTet_Lac, pLac/Mnt, pTet/Mnt, LsrA/cl, pLux/cl, LacI, LacIQ, pLacIQI, pLas/cl, pLas/Lux, pLux/Las, pRecA with LexA binding site, reverse BBa_R0011, pLacI/ara-1, pLacIq, rrnB PI, cadC, hns, PfhuA, pBad/araC, nhaA, OmpF, RcnR), σ S promoters (e.g., Lutz-Bujard LacO with alternative sigma factor σ 38), σ 32 promoters (e.g., Lutz-Bujard LacO with alternative sigma factor σ 32), a 54 promoters (e.g., glnAp2); negatively regulated B. subtilis promoters such as repressible B. subtilis σ A promoters (e.g., Gram-positive IPTG-inducible, Xyl, hyper-spank), a promoters, and the BioFAB promoters disclosed in Mutalik V K et al (Nature Methods, 2013, 10: 354-360, see in particular the supplementary data) as well as on the BioFAB website (http://biofab.synberc.org/data). Other inducible microbial promoters and/or bacterial promoters may be used in accordance with the present invention. An inducible promoter for use in accordance with the present disclosure may be induced by (or repressed by) one or more physiological condition(s), such as changes in pH, temperature, radiation, osmotic pressure, saline gradients, cell surface binding, and the concentration of one or more extrinsic or intrinsic inducing agent(s). The extrinsic inducer or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Particularly preferred bacterial promoters for use in accordance with the present invention may be selected from constitutive promoters regulated by α70 such as the promoters of the Anderson collection (http://parts.igem.org/Promoters/Catalog/Anderson): BBa_J23100, BBa_J23101, BBa_J23102, BBa_J23103, BBa_J23104, BBa_J23105, BBa_J23106, BBa_J23107, BBa_J23108, BBa_J23109, BBa_J23110, BBa_J23111, BBa_J23112, BBa_J23113, BBa_J23114, BBa_J23115, BBa_J23116, BBa_J23117, BBa_J23118, and BBa_J23119.

Other preferred bacterial promoters are the promoters disclosed in Stanton et al. (2014) Nat. Chem. Biol. 10:99-105, incorporated herein by reference, including in particular TetR, IcaR(A), AmtR, BetI, SrpR, Orf2, BM3R1, ButR, PhlF, PsrA, HlyIIR, AmeR, LmrA, QacR, ScbR, McbR, LitR, HapR, SmcR, TarA and variants thereof. In a particular embodiment, said promoter is SrpR and/or PhlF, or a variant thereof.

In some embodiments of the present invention, a promoter may or may not be used in conjunction with an “enhancer,” which refers to a ds-acting regulatory sequence involved in the transcriptional activation of a nucleic acid sequence downstream of the promoter. The enhancer may be located at any functional location before or after the promoter.

In some embodiments, the vector may comprise a terminator sequence, or terminator. A “terminator,” as used herein, is a nucleic acid sequence that causes transcription to stop. A terminator may be unidirectional or bidirectional. It is comprised of a DNA sequence involved in specific termination of an RNA transcript by an RNA polymerase. A terminator sequence prevents transcriptional activation of downstream nucleic acid sequences by upstream promoters. Thus, in certain embodiments, a terminator that ends the production of an RNA transcript is contemplated. A terminator may be necessary in vivo to achieve desirable gene/protein expression levels.

The most commonly used type of terminator is a forward terminator. When placed downstream of a nucleic acid of interest that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only. In prokaryotic systems, terminators usually fall into two categories (1) rho-independent terminators and (2) rho-dependent terminators. Rho-independent terminators are generally composed of palindromic sequence that forms a stem loop rich in G-C base pairs followed by a string of uracil bases.

Terminators for use in accordance with the present invention include any terminator of transcription described herein or known to one of ordinary skill in the art. Examples of terminators include, without limitation, the termination sequences of genes such as, for example, the bovine growth hormone terminator, and viral termination sequences such as, for example, the TO terminator, the TE terminator, lambda TI and the T1T2 terminator found in bacterial systems. In some embodiments, the termination signal may be a sequence that cannot be transcribed or translated, such as those resulting from a sequence truncation.

Terminators for use in accordance with the present invention also include terminators disclosed in Chen Y J et al (2013, Nature Methods, 10: 659-664), and the BioFAB terminators disclosed in Cambray G et al (Nucl Acids Res, 2013, 41(9): 5139-5148).

### Vector

As used herein, the term “vector” refers to a nucleic acid molecule, typically DNA or RNA that serves to transfer a passenger nucleic acid sequence, i.e. DNA or RNA, into a receiver or target cell. A vector may comprise an origin of replication, a selectable marker, and optionally a suitable site for the insertion of a gene such as the multiple cloning site. There are several common types of vectors including plasmids, bacteriophage genomes, phagemids, phage-plasmids, virus genomes, cosmids, and artificial chromosomes.

In the context of the invention, a vector may be referred to as a payload.

The vector used in the context of the invention may be a plasmid (e.g, a conjugative plasmid capable of transfer into a host cell), phage, phagemid or prophage.

The payload can be a phagemid or phasmid obtained from natural, evolved or engineered bacteriophage genome. The payload can also be composed only in part of phagemid or phasmid obtained from natural, evolved or engineered bacteriophage genome.

In some embodiments, the payload is the delivery vehicle as bacteria are naturally competent to take up a payload from the environment on their own.

As used herein, the terms “phagemid” and “phasmid” are equivalent and refer to a vector that derives from both a plasmid and a bacteriophage genome. A phagemid of the disclosure comprises a phage packaging site and an origin of replication (ori), as disclosed below.

As used herein, the term “packaged phagemid” refers to a phagemid which is encapsidated in a bacteriophage scaffold, bacterial virus particle or capsid. Particularly, it refers to a bacteriophage scaffold, bacterial virus particle or capsid devoid of a bacteriophage genome. The packaged phagemid may be produced with a helper phage strategy, well known from the man skilled in the art. The helper phage comprises all the genes coding for the structural and functional proteins that are indispensable for the phagemid according to the invention to be encapsidated. The packaged phagemid may be produced with a satellite virus strategy, also known from the man skilled in the art. Satellite virus are subviral agent and are composed of nucleic acid that depends on the co-infection of a host cell with a helper virus for all the morphogenetic functions, whereas for all its episomal functions (integration and immunity, multicopy plasmid replication) the satellite is completely autonomous from the helper. In one embodiment, the satellite genes can encode proteins that promote capsid size reduction of the helper phage, as described for the P4 Sid protein that controls the P2 capsid size to fit its smaller genome.

In a particular embodiment, when said vector is a packaged phagemid, said vector does not comprise any element derived from the organism from which the conditional origin of replication is derived. In particular, the packaging site of said vector is not derived from the organism from which the conditional origin of replication is derived.

Vectors can include, without limitation, plasmid vectors and recombinant phage vectors. The skilled artisan is well aware of the genetic elements that must be present on the vector in order to successfully transform and select host cells comprising any of the isolated nucleotides or nucleic acid sequences of the invention.

As used herein, the term “conjugative plasmid” refers to a plasmid that is transferred from one bacterial cell to another during conjugation and a “donor bacterium”, as used herein, is then a bacterium that is capable of transferring a conjugative plasmid to another bacterium.

The vector used in the context of the invention is devoid of antibiotic resistance marker.

Antibiotic resistance genes are well known in the art and include but are not limited to ampicillin resistance (Amp), chloramphenicol resistance (Cm), tetracycline resistance (Tet), kanamycin resistance (Kan), hygromycin resistance (Qiyg or hph genes), and zeomycin resistance (Zeo).

In a particular embodiment, the vector used in the context of the invention comprises an auxotrophic marker. Auxotrophic markers in bacteria have previously been described, for example, in U.S. Pat. Nos. 4,920,048, 5,691,185, 6,291,245, 6,413,768, and 6,752,994; U.S. Patent Publication No. 20050186666; Struhl et al. (1976) PNAS USA 73; 1471-1475; MacCormick et al., (1995) FEMS Microbiol. Lett. 127:105-109; Dickely et al. (1995) Mol. Microbiol. 15:839-847; Sorensen et al. (2000) Appl. Environ. Microbiol 66:1253-1258; and Fiedler & Skerra (2001) Gene 274: 111 118, all incorporated herein by reference, and typically include DapA and ThyA. In a particular embodiment, said auxotrophic marker is ThyA.

In a particular embodiment, said vector does not comprise any restriction site recognized by restriction enzymes which are frequently encoded by said targeted receiver bacterial cell. In another particular embodiment, said vector comprises no more than 40, 30, 20, 10, 9, 8, 7, 6, 5, 4, 3, 2 or 1 restriction site(s) recognized by restriction enzymes which are frequently encoded by said targeted bacterial cell or a population or a group of targeted bacterial cell(s).

As used herein, the terms “restriction site” and “restriction enzyme site” are equivalent and refer to locations on a nucleic acid containing specific sequences of nucleotides, which are recognized by restriction enzymes. In particular, the nucleic acid comprises specific sequences which are bound and cleaved by restriction enzymes. Restriction sites are generally palindromic sequences of 4-8 base pairs in length. More precisely, the restriction site refers to a particular sequence and a modification state, so as to be bound and cleaved by restriction enzymes. In particular, it refers to a particular unmodified sequence, so as to be bound and cleaved by restriction enzymes. Especially the sequence is not methylated, hydroxymethylated and glucosyl-hydroxymethylated. In this context, the restriction enzyme is of type I, II or III. Alternatively, it may refer to a particular modified sequence, so as to be bound and cleaved by restriction enzymes, for instance a methylated, hydroxymethylated and glucosyl-hydroxymethylated DNA. In this context, the restriction enzyme is of type IV.

As used herein, “recognized by” with respect to a restriction site and a restriction enzyme means that the restriction site is cleaved by the restriction enzyme.

In a restriction site sequence N means that the nucleotide can be A, C, G or T; B means that the nucleotide can be C, G or T; Y means that the nucleotide can be C or T; W means that the nucleotide can be A or T; R means that the nucleotide can be A or G; and D means A, G or T.

As used herein, the terms “restriction enzyme” and “restriction endonuclease” are equivalent and refer to an enzyme that cuts nucleic acids at or near restriction sites. Restriction enzymes are commonly classified into four types (types I to type IV). The REBASE database allow to list the restriction sites that a given bacterium can recognize according to the restriction enzymes that it expresses.

By “frequent” or “frequently” in a group of bacteria of interest is meant that at least 10, 20, 30, 40, 50, 60, 70, 75, 80, 85, 90, 95 or 99% of the bacteria of the group encode the restriction enzyme.

The vector according to the invention, preferably included into a delivery vehicle, preferably a bacteriophage capsid, preferably comprises no more than 100 restriction sites. In a preferred embodiment, the vector according to the invention, preferably included in a delivery vehicle, comprises no more than 10 restriction sites. In a most preferred embodiment, the vector according to the invention, preferably included in a delivery vehicle, does not comprise any restriction site.

The present invention also concerns a nucleic acid vector, as defined above, for use in in vivo delivery of a nucleic acid of interest, as defined above, into a targeted receiver bacterial cell, said nucleic acid of interest producing a given effect on said targeted receiver bacterial cell,

wherein said vector comprises:
said nucleic acid of interest, as defined above, and
a conditional origin of replication which is inactive in the targeted receiver bacterial cell but is active in a donor bacterial cell, and

wherein said vector is devoid of antibiotic resistance marker.

### Conditional Origin of Replication

The vector of the invention comprises a conditional origin of replication which is inactive in the targeted receiver bacterial cell but is active in a donor bacterial cell.

In the context of the invention, a “conditional origin of replication” refers to an origin of replication whose functionality may be controlled by the presence of a specific molecule.

In a particular embodiment, the conditional origin of replication is an origin of replication, the replication of which depends upon the presence of one or more given protein, peptid, RNA, nucleic acid, molecule or any combination thereof.

In a particular embodiment, the replication of said origin of replication may further depend on a process, such as transcription, to activate said replication.

In the context of the invention, said conditional origin of replication is inactive in the targeted receiver bacterial cell because of the absence of said given protein, peptid, RNA, nucleic acid, molecule or any combination thereof in said receiver bacterial cell.

In a particular embodiment, said conditional origin of replication is active in said donor bacterial cell because said donor bacterial cell expresses said given protein, peptid, RNA, nucleic acid, molecule or any combination thereof. In a particular embodiment, said protein, peptid, RNA nucleic acid, molecule or any combination thereof is expressed in trans in said donor bacterial cell.

By “in trans” is meant herein that said protein, peptid, RNA, nucleic acid, molecule or any combination thereof is not encoded on the same nucleic acid molecule as the one comprising the origin of replication. In a particular embodiment, said protein, peptid, RNA, nucleic acid, molecule or any combination thereof is encoded on a chromosome or on a plasmid. In a particular embodiment, said plasmid comprises an antibiotic resistance marker. In an alternative embodiment, said plasmid is devoid of antibiotic resistance marker.

Since said conditional origin of replication is inactive in the targeted receiver bacterial cell because of the absence of said given protein, peptid, RNA, nucleic acid, molecule or any combination thereof in said receiver bacterial cell, said conditional origin of replication may be selected depending on the specific receiver bacterial cell to be targeted.

The conditional origin of replication used according to the present invention may originate from plasmids, bacteriophages or PICIs which preferably share the following characteristics: they contain in their origin of replication repeat sequences, or iterons, and they code for at least one protein interacting with said origin of replication (i.e. Rep, protein O, protein P, pri) which is specific to them.

By way of example, mention may be made of the conditional replication systems of the following plasmids and bacteriophages: RK2, R1, pSC101, F, Rts1, RSF1010, P1, P4, lambda, phi82, phi80.

In a particular embodiment, said conditional origin of replication is selected from the group consisting of the R6Kλ DNA replication origin and derivatives thereof, the IncPa oriV origin of replication and derivatives thereof, ColE1 origins of replication modified to be under an inducible promoter, and origins of replication from phage-inducible chromosomal islands (PICIs) and derivatives thereof.

In a particular embodiment, said conditional origin of replication is an origin of replication present in less than 50%, or less than 40%, less than 30%, less than 20%, less than 10% or less than 5% of the bacteria of the host microbiome.

In another particular embodiment, said conditional origin of replication comprises or consists of a sequence less than 80% identical, in particular less than 70%, less than 60%, less than 50%, less than 40%, less than 30%, less than 20%, less than 10%, less than 5% or less than 1% identical to the sequences of the origins of replication of the bacteria of the host microbiome, in particular of the bacteria representing more than 50%, more particularly more than 60%, more than 70%, more than 80%, more than 90% or more than 95% of the host microbiome.

In the context of the invention, the term “phaqe-inducible chromosomal islands” or “PICIs” are mobile genetic elements having a conserved gene organization, and encode a pair of divergent regulatory genes, including a PICI master repressor. Typically, in Gram-positive bacteria, left of rpr, and transcribed in the same direction, PICIs encode a small set of genes including an integrase (int) gene; right of rpr, and transcribed in the opposite direction, the PICIs encode an excision function (xis), and a replication module consisting of a primase homolog (pri) and optionally a replication initiator (rep), which are sometimes fused, followed by a replication origin (ori), next to these genes, and also transcribed in the same direction, PICIs encode genes involved in phage interference, and optionally, a terminase small subunit homolog (terS).

In a particular embodiment, said conditional origin of replication is an origin of replication derived from phage-inducible chromosomal islands (PICIs).

The present inventors indeed designed herein a particular conditional origin of replication derived from PICIs.

The present inventors showed that it is possible to derive novel conditionally replicative plasmids, in particular based on the primase-helicase and origin of replication from PICIs. These origins may be relatively rare in target strains, and more advantageously the primase-ori pair may be unique for each PICI, significantly reducing the possibility of undesired recombination or payload spread events. They can further be modified to further limit recombination chances and remove restriction sites to bypass target bacteria defense systems.

In a particular embodiment, said conditional origin of replication is derived from the origin of replication from the PICI of the Escherichia coli strain CFT073, disclosed in Fillol-Salom et al. (2018) The ISME Journal 12:2114-2128.

In a particular embodiment, said conditional origin of replication is the primase ori from the PICI of the Escherichia coli strain CFT073, typically of sequence SEQ ID NO: 4.

In another particular embodiment, said conditional origin of replication is the primase ori from he PICI of the Escherichia coli strain CFT073, devoid of at least 1, at least 2, at least 3, at least 4, at least 5, at least 6, at least 7, at least 8, at least 9, at least 10, at least 11, at least 12, at least 13, at least 14, at least 15 or at least 16 restriction site(s) selected from the group consisting of GAAABCC, GCCGGC, RCCGGY, GCNGC, TWCANNNNNNTGG (SEQ ID NO: 5), TGGCCA, ACCYAC, YGGCCR, AGACC, GCWGC, GGGANGC, GKAGATD, GCCGGYYD, GGCYAC, RGCCGGYYD, and VGCCGGYBD.

In a particular embodiment, said conditional origin of replication is the primase ori from the PICI of the Escherichia coli strain CFT073, devoid of the restriction site GAAABCC. Preferably, said conditional origin of replication is of sequence SEQ ID NO: 6.

In another particular embodiment, said conditional origin of replication is the primase ori from he PICI of the Escherichia coli strain CFT073 devoid of the restriction sites selected from the group consisting of GAAABCC, GCCGGC, RCCGGY, GCNGC, TWCANNNNNNTGG (SEQ ID NO: 5), TGGCCA, ACCYAC, YGGCCR, AGACC, GCWGC, GGGANGC, GKAGATD, GCCGGYYD, GGCYAC, RGCCGGYYD, and VGCCGGYBD. Preferably, said conditional origin of replication is of sequence SEQ ID NO: 7.

In a particular embodiment, wherein said origin of replication is derived from phage-inducible chromosomal islands (PICIs), said conditional origin of replication is active in said donor bacterial cell because said donor bacterial cell expresses a rep protein, in particular a primase-helicase, in particular a primase-helicase of sequence SEQ ID NO: 8, typically encoded by a nucleic acid comprising or consisting of the sequence SEQ ID NO: 9.

The inventors demonstrated that these specific conditional origins of replication were particularly compatible with lambda-based packaging, leading to sufficiently high titers (>10¹⁰/mL) required for microbiota-related applications.

In a particular embodiment, the vector of the invention comprises or consists of the sequence SEQ ID NO: 10. In another particular embodiment, the vector of the invention comprises or consists of the sequence SEQ ID NO: 11.

In a particular embodiment, when said vector is a phagemid, said origin of replication may be derived from a microorganism which is different from the one that is used to encode the structural elements of the capsid packaging said phagemid.

### Bacterial Delivery Vehicle

In a particular embodiment, said vector is located inside a bacterial delivery vehicle. Preferably, the vector located inside a delivery vehicle is a phagemid and the delivery vehicle is a bacterial virus particle or a capsid.

As used herein, the term «delivery vehicle» refers to any vehicle that allows the transfer of a vector or payload into a bacterium.

There are several types of delivery vehicle encompassed by the present invention including, without limitation, bacteriophage scaffold, virus scaffold, bacterial virus particle, chemical based delivery vehicle (e.g., cyclodextrin, calcium phosphate, cationic polymers, cationic liposomes), protein-based or peptide-based delivery vehicle, lipid-based delivery vehicle, nanoparticle-based delivery vehicles, non-chemical-based delivery vehicles (e.g., transformation, electroporation, sonoporation, optical transfection), particle-based delivery vehicles (e.g., gene gun, magnetofection, impalefection, particle bombardment, cell-penetrating peptides) or donor bacteria (conjugation).

Any combination of delivery vehicles is also encompassed by the present invention.

The delivery vehicle can refer to a bacteriophage derived scaffold and can be obtained from a natural, evolved or engineered capsid.

In some embodiments, the delivery vehicle is the vector or payload as bacteria are naturally competent to take up a payload from the environment on their own.

The present disclosure is directed to a bacterial delivery vehicle containing the vector or payload as described herein. The bacterial delivery vehicles are typically prepared from bacterial virus. The bacterial delivery vehicles are typically chosen in order to be able to introduce the vector into the targeted bacteria.

Bacterial viruses, from which the bacterial delivery vehicles disclosed herein may be derived, include bacteriophages. Optionally, the bacteriophage is selected from the Order Caudovirales consisting of, based on the taxonomy of Krupovic et al, Arch Virol, 2015, the family Myoviridae, the family Podoviridae, the family Siphoviridae, and the family Ackermannviridae.

Bacteriophages may be selected from the family Myoviridae (such as, without limitation, genus Cp220virus, Cp8virus, Ea214virus, Felixo1virus, Mooglevirus, Suspvirus, Hp1virus, P2virus, Kayvirus, P100virus, Silviavirus, Spo1virus, Tsarbombavirus, Twortvirus, Cc31virus, Jd18virus, Js98virus, Kp15virus, Moonvirus, Rb49virus, Rb69virus, S16virus, Schizot4virus, Sp18virus, T4virus, Cr3virus, Se1virus, V5virus, Abouovirus, Agatevirus, Agrican357virus, Ap22virus, Arv1virus, B4virus, Bastillevirus, Bc431virus, Bcep78virus, Bcepmuvirus, Biquartavirus, Bxz1virus, Cd119virus, Cp51virus, Cvm10virus, Eah2virus, Elvirus, Hapunavirus, Jimmervirus, Kpp10virus, M12virus, Machinavirus, Marthavirus, Msw3virus, Muvirus, Myohalovirus, Nit1virus, P1virus, Pakpunavirus, Pbunavirus, Phikzvirus, Rheph4virus, Rsl2virus, Rslunavirus, Secunda5virus, Sep1virus, Spn3virus, Svunavirus, Tg1virus, Vhmlvirus and Wphvirus).

Bacteriophages may be selected from the family Podoviridae (such as, without limitation, genus Fri1virus, Kp32virus, Kp34virus, Phikmvvirus, Pradovirus, Sp6virus, T7virus, Cp1virus, P68virus, Phi29virus, Nona33virus, Pocjvirus, T12011virus, Bcep22virus, Bpp1virus, Cba41virus, Dfl12virus, Ea92virus, Epsilon15virus, F116virus, G7cvirus, Jwalphavirus, Kf1virus, Kpp25virus, Lit1virus, Luz24virus, Luz7virus, N4virus, Nonanavirus, P22virus, Pagevirus, Phieco32virus, Prtbvirus, Sp58virus, Una961virus and Vp5virus).

Bacteriophages may be selected from the family Siphoviridae (such as, without limitation, genus Camvirus, Likavirus, R4virus, Acadianvirus, Coopervirus, Pg1virus, Pipefishvirus, Rosebushvirus, Brujitavirus, Che9cvirus, Hawkeyevirus, Plotvirus, Jerseyvirus, K1gvirus, Sp31virus, Lmd1virus, Una4virus, Bongovirus, Reyvirus, Buttersvirus, Charlievirus, Redivirus, Baxtervirus, Nymphadoravirus, Bignuzvirus, Fishburnevirus, Phayoncevirus, Kp36virus, Rogue1virus, Rtpvirus, T1virus, Tlsvirus, Ab18virus, Amigovirus, Anatolevirus, Andromedavirus, Attisvirus, Barnyardvirus, Bernal13virus, Biseptimavirus, Bronvirus, C2virus, C5virus, Cba181virus, Cbastvirus, Cecivirus, Che8virus, Chivirus, Cjw1virus, Corndogvirus, Cronusvirus, D3112virus, D3virus, Decurrovirus, Demosthenesvirus, Doucettevirus, E125virus, Eiauvirus, Ff47virus, Gaiavirus, Gilesvirus, Gordonvirus, Gordtnkvirus, Harrisonvirus, Hk578virus, Hk97virus, Jenstvirus, Jwxvirus, Kelleziovirus, Korravirus, L5virus, lambdavirus, Laroyevirus, Liefievirus, Marvinvirus, Mudcatvirus, N15virus, Nonagvirus, Np1virus, Omegavirus, P12002virus, P12024virus, P23virus, P70virus, Pa6virus, Pamx74virus, Patiencevirus, Pbi1virus, Pepy6virus, Pfr1virus, Phic31virus, Phicbkvirus, Phietavirus, Phifelvirus, Phijl1virus, Pis4avirus, Psavirus, Psimunavirus, Rdjlvirus, Rer2virus, Sap6virus, Send513virus, Septima3virus, Seuratvirus, Sextaecvirus, Sfi11virus, Sfi21dt1virus, Sitaravirus, Sk1virus, Slashvirus, Smoothievirus, Soupsvirus, Spbetavirus, Ssp2virus, T5virus, Tankvirus, Tin2virus, Titanvirus, Tm4virus, Tp21virus, Tp84virus, Triavirus, Trigintaduovirus, Vegasvirus, Vendettavirus, Wbetavirus, Wildcatvirus, Wizardvirus, Woesvirus, Xp10virus, Ydn12virus and Yuavirus).

Bacteriophages may be selected from the family Ackermannviridae (such as, without limitation, genus Ag3virus, Limestonevirus, Cba120virus and Vi1virus).

Optionally, the bacteriophage is not part of the order Caudovirales but from families with unassigned order such as, without limitation, family Tectiviridae (such as genus Alphatectivirus, Betatectivirus), family Corticoviridae (such as genus Corticovirus), family Inoviridae (such as genus Fibrovirus, Habenivirus, Inovirus, Lineavirus, Plectrovirus, Saetivirus, Vespertiliovirus), family Cystoviridae (such as genus Cystovirus), family Leviviridae (such as genus Allolevivirus, Levivirus), family Microviridae (such as genus Alpha3microvirus, G4microvirus, Phix174microvirus, Bdellomicrovirus, Chlamydiamicrovirus, Spiromicrovirus) and family Plasmaviridae (such as genus Plasmavirus).

Optionally, the bacteriophage is targeting Archea not part of the Order Caudovirales but from families with unassigned order such as, without limitation, Ampullaviridae, FuselloViridae, Globuloviridae, Guttaviridae, Lipothrixviridae, Pleolipoviridae, Rudiviridae, Salterprovirus and Bicaudaviridae.

A non-exhaustive listing of bacterial genera and their known host-specific bacteria viruses is presented in the following paragraphs. Synonyms and spelling variants are indicated in parentheses. Homonyms are repeated as often as they occur (e.g., D, D, d). Unnamed phages are indicated by “NN” beside their genus and their numbers are given in parentheses.

Bacteria of the genus Actinomyces can be infected by the following phages: Av-1, Av-2, Av-3, BF307, CTI, CT2, CT3, CT4, CT6, CT7, CT8 and 1281.

Bacteria of the genus Aeromonas can be infected by the following phages: AA-, Aeh2, N, PMI, TP446, 3, 4, 11, 13, 29, 31, 32, 37, 43, 43-10T, 51, 54, 55R.1, 56, 56RR2, 57, 58, 59.1, 60, 63, AehI, F, PM2, 1, 25, 31, 40RR2.8t, (syn=44R), (syn=44RR2.8t), 65, PM3, PM4, PM5 and PM6.

Bacteria of the genus Bacillus can be infected by the following phages: A, aizI, AI-K-I, B, BCJAI, BCI, BC2, BLLI, BLI, BP142, BSLI, BSL2, BSI, BS3, BS8, BS15, BS18, BS22, BS26, BS28, BS31, BS104, BS105, BS106, BTB, B1715V1, C, CK-1, ColI, CorI, CP-53, CS-1, CSi, D, D, D, D5, entI, FP8, FP9, FSi, FS2, FS3, FS5, FS8, FS9, G, GH8, GT8, GV-1, GV-2, GT-4, g3, g12, g13, g14, g16, g17, g21, g23, g24, g29, H2, kenI, KK-88, KumI, KyuI, J7W-1, LP52, (syn=LP-52), L7, MexI, MJ-I, mor2, MP-7, MPIO, MP12, MP14, MP15, NeoI, N^{o}2, N5, N6P, PBCI, PBLA, PBPI, P2, S-a, SF2, SF6, ShaI, SilI, SP02, (syn=ΦSPP1), SPβ, STI, STi, SU-II, t, TbI, Tb2, Tb5, TblO, Tb26, Tb51, Tb53, Tb55, Tb77, Tb97, Tb99, Tb560, Tb595, Td8, Td6, Tdl5, TgI, Tg4, Tg6, Tg7, Tg9, TglO, TglI, Tgl3, Tg15, Tg21, TinI, Tin7, Tin8, Tin13, Tm3, TocI, TogI, tolI, TP-1, TP-10vir, TP-15c, TP-16c, TP-17c, TP-19, TP35, TP51, TP-84, Tt4, Tt6, type A, type B, type C, type D, type E, TP3, VA-9, W, wx23, wx26, YunI, α, γ, pl I, φmed-2, φT, pp-4, φ3T, φ75, φIO5, (syn=φIO5), IA, IB, 1-97A, 1-97B, 2, 2, 3, 3, 3, 5, 12, 14, 20, 30, 35, 36, 37, 38, 41C, 51, 63, 64, 138D, 1, 11, IV, NN-Bacillus (13), aleI, ARI, AR2, AR3, AR7, AR9, Bace-11, (syn=11), Bastille, BLI, BL2, BL3, BL4, BL5, BL6, BL8, BL9, BP124, BS28, BS80, Ch, CP-51, CP-54, D-5, darI, denI, DP-7, entI, FoSi, FoS2, FS4, FS6, FS7, G, gall, gamma, GEI, GF-2, GSi, GT-I, GT-2, GT-3, GT-4, GT-5, GT-6, GT-7, GV-6, g15, 19, 110, ISi, K, MP9, MP13, MP21, MP23, MP24, MP28, MP29, MP30, MP32, MP34, MP36, MP37, MP39, MP40, MP41, MP43, MP44, MP45, MP47, MP50, NLP-I, No.I, N17, N19, PBSI, PKI, PMBI, PMB12, PMJI, S, SPOI, SP3, SP5, SP6, SP7, SP8, SP9, SPIO, SP-15, SP50, (syn=SP-50), SP82, SST, subI, SW, Tg8, Tg12, Tgl3, Tg14, thuI, thuA, thuS, Tin4, Tin23, TP-13, TP33, TP50, TSP-I, type V, type VI, V, Vx, β22, cpe, pNR2, φ25, φ63, 1, 1, 2, 2C, 3NT, 4, 5, 6, 7, 8, 9, 10, 12, 12, 17, 18, 19, 21, 138, 111, 4 (B. megateriwn), 4 (B. sphaericus), AR13, BPP-IO, BS32, BS107, BI, B2, GA-1, GP-IO, GV-3, GV-5, g8, MP20, MP27, MP49, Nf, PP5, PP6, SF5, Tgl8, TP-1, Versailles, φ15, φ29, 1-97, 837/IV, mï-Bacillus (1), BatIO, BSLIO, BSLI 1, BS6, BSI 1, BS16, BS23, BSIOI, BS102, g18, morI, PBLI, SN45, thu2, thu3, Tml, Tm2, TP-20, TP21, TP52, type F, type G, type IV, HN-BacMus (3), BLE, (syn=θc), BS2, BS4, BS5, BS7, BIO, B12, BS20, BS21, F, MJ-4, PBA12, AP50, AP50-04, AP50-11, AP50-23, AP50-26, AP50-27 and Bam35. The following Bacillus-specific phages are defective: DLP10716, DLP-11946, DPB5, DPB12, DPB21, DPB22, DPB23, GA-2, M, No. IM, PBLB, PBSH, PBSV, PBSW, PBSX, PBSY, PBSZ, phi, SPa, type 1 and μ.

Bacteria of the genus Bacteroides can be infected by the following phages: ad I2, Baf-44, Baf-48B, Baf-64, Bf-1, Bf-52, B40-8, FI, βI, φAI, φBrOI, φBrO2, 11, 67.1, 67.3, 68.1, mt-Bacteroides (3), Bf42, Bf71, HN-Bdellovibrio (1) and BF-41.

Bacteria of the genus Bordetella can be infected by the following phages: 134 and NN-Bordetella (3).

Bacteria of the genus Borrelia can be infected by the following phages: NN-Borrelia (1) and NN-Borrelia (2).

Bacteria of the genus Brucella can be infected by the following phages: A422, Bk, (syn=Berkeley), BM29, FOi, (syn=FOI), (syn=FQI), D, FP2, (syn=FP2), (syn=FD2), Fz, (syn=Fz75/13), (syn=Firenze 75/13), (syn=Fi), Fi, (syn=FI), Fim, (syn=Flm), (syn=Fim), FiU, (syn=FIU), (syn=FiU), F2, (syn=F2), F3, (syn=F3), F4, (syn=F4), F5, (syn=F5), F6, F7, (syn=F7), F25, (syn=F25), (syn=£25), F25U, (syn=F25u), (syn=F25U), (syn=F25V), F44, (syn-F44), F45, (syn=F45), F48, (syn=F48), I, Im, M, MC/75, M51, (syn=M85), P, (syn=D), S708, R, Tb, (syn=TB), (syn=Tbilisi), W, (syn=Wb), (syn=Weybridge), X, 3, 6, 7, 10/1, (syn=10), (syn=F8), (syn=F8), 12m, 24/11, (syn=24), (syn=F9), (syn=F9), 45/111, (syn=45), 75, 84, 212/XV, (syn=212), (syn=Fi0), (syn=FIO), 371/XXIX, (syn=371), (syn=Fn), (syn=FI I) and 513.

Bacteria of the genus Burkholderia can be infected by the following phages: CP75, NN-Burkholderia (1) and 42.

Bacteria of the genus Campylobacter can be infected by the following phages: C type, NTCC12669, NTCC12670, NTCC12671, NTCC12672, NTCC12673, NTCC12674, NTCC12675, NTCC12676, NTCC12677, NTCC12678, NTCC12679, NTCC12680, NTCC12681, NTCC12682, NTCC12683, NTCC12684, 32f, 111c, 191, NN-Campylobacter (2), Vfi-6, (syn=V19), VfV-3, V2, V3, V8, V16, (syn=Vfi-1), V19, V20(V45), V45, (syn=V-45) and NN-Campylobacter (1).

Bacteria of the genus Chlamydia can be infected by the following phages: ChpI.

Bacteria of the genus Clostridium can be infected by the following phages: CAKI, CA5, Ca7, CEβ, (syn=1C), CEγ, CldI, c-n71, c-203 Tox-, DEβ, (syn=ID), (syn=IDt0X+), HM3, KMI, KT, Ms, NAI, (syn=Naltox+), PA135Oe, Pfó, PL73, PL78, PL81, PI, P50, P5771, P19402, ICt0X+, 2Ct0X\ 2D3 (syn=2Dt0X+), 3C, (syn=3Ctox+), 4C, (syn=4Ct0X+), 56, III-I, NN-Clostridium (61), NBlt0X+, αI, CAI, HMT, HM2, PFI5 P-23, P-46, Q-05, Q-oe, Q-16, Q-21, Q-26, Q-40, Q-46, S111, SA02, WA01, WA03, Wm, W523, 80, C, CA2, CA3, CPTI, CPT4, cl, c4, c5, HM7, H11/A1, H18/Ax, FWS23, Hi58ZA1, K2ZA1, K21ZS23, ML, NA2t0X; Pf2, Pf3, Pf4, S9ZS3, S41ZA1, S44ZS23, α2, 41, 112ZS23, 214/S23, 233/Ai, 234/S23, 235/S23, II-I, II-2, II-3, NN-Clostridium (12), CAI, FI, K, S2, 1, 5 and NN-Clostridium (8).

Bacteria of the genus Corynebacterium can be infected by the following phages: CGKI (defective), A, A2, A3, AIOI, A128, A133, A137, A139, A155, A182, B, BF, B17, B18, B51, B271, B275, B276, B277, B279, B282, C, capi, CCI, CGI, CG2, CG33, CL31, Cog, (syn=CG5), D, E, F, H, H-I, hqi, hq2, 11ZH33, Ii/31, J, K, K, (syn=Ktox″), L, L, (syn=Ltox+), M, MC-I, MC-2, MC-3, MC-4, MLMa, N, O, ovi, ov2, ov3, P, P, R, RP6, RS29, S, T, U, UB1, ub2, UH1, UH3, uh3, uh5, uh6, β, (syn=βtox+), βhv64, βvir, γ, (syn=γtoχ−), γI9, δ, (syn=δ′ox+), p, (syn=ptoχ−), D9, φ984, ω, IA, 1/1180, 2, 2/1180, 5/1180, 5ad/9717, 7/4465, 8/4465, 8ad/10269, 10/9253, 13Z9253, 15/3148, 21/9253, 28, 29, 55, 2747, 2893, 4498 and 5848.

Bacteria of the genus Enterococcus can be infected by the following phages: DF78, FI, F2, 1, 2, 4, 14, 41, 867, DI, SB24, 2BV, 182, 225, C2, C2F, E3, E62, DS96, H24, M35, P3, P9, SBIOI, S2, 2BII, 5, 182a, 705, 873, 881, 940, 1051, 1057, 21096C, NN-Enterococcus (1), PEI, FI, F3, F4, VD13, 1, 200, 235 and 341.

Bacteria of the genus Erysipelothrix can be infected by the following phage: NN-Eiysipelothrix (1).

Bacteria of the genus Escherichia can be infected by the following phages: BW73, B278, D6, D108, E, EI, E24, E41, FI-2, FI-4, FI-5, HI8A, Ffl8B, i, MM, Mu, (syn=mu), (syn=MuI), (syn=Mu-I), (syn=MU-I), (syn=MuI), (syn=p), 025, Phl-5, Pk, PSP3, PI, PID, P2, P4 (defective), SI, Wφ, φK13, φR73 (defective), φ1, φ2, φ7, φ92, ψ (defective), 7 A, 8φ, 9φ, 15 (defective), 18, 28-1, 186, 299, HH-Escherichia (2), AB48, CM, C4, C16, DD-VI, (syn=Dd-Vi), (syn=DDVI), (syn=DDVi), E4, E7, E28, FII, FI3, H, HI, H3, H8, K3, M, N, ND-2, ND-3, ND4, ND-5, ND6, ND-7, Ox-I (syn=OXI), (syn=HF), Ox-2 (syn=0x2), (syn=0X2), Ox-3, Ox-4, Ox-5, (syn=0X5), Ox-6, (syn=66F), (syn=φ66t), (syn=φ66t-)5 0111, Phl-I, RB42, RB43, RB49, RB69, S, Sal-I, Sal-2, Sal-3, Sal-4, Sal-5, Sal-6, TC23, TC45, TulI*-6, (syn=TulI*), TulP-24, TulI*46, TuIP-60, T2, (syn=ganuTia), (syn=γ), (syn=PC), (syn=P.C.), (syn=T-2), (syn=T2), (syn=P4), T4, (syn=T-4), (syn=T4), T6, T35, αI, 1, IA, 3, (syn=Ac3), 3A, 3T+, (syn=3), (syn=MI), 5φ, (syn=φ5), 9266Q, CFO103, HK620, J, K, KIF, m59, no. A, no. E, no. 3, no. 9, N4, sd, (syn=Sd), (syn=SD), (syn=Sa)3 (syn=sd), (syn=SD), (syn=CD), T3, (syn=T-3), (syn=T3), T7, (syn=T-7), (syn=T7), WPK, W31, ΔH, φC3888, φK3, φK7, φK12, φV-1, Φ04-CF, Φ05, Φ06, Φ07, φI, φI.2, φ20, φ95, φ263, φIO92, φI, φII, (syn=φW), Ω8, 1, 3, 7, 8, 26, 27, 28-2, 29, 30, 31, 32, 38, 39, 42, 933W, NN-Escherichia (1), Esc-7-11, AC30, CVX-5, CI, DDUP, ECI, EC2, E21, E29, FI, F26S, F27S, Hi, HK022, HK97, (syn=ΦHK97), HK139, HK253, HK256, K7, ND-I, no.D, PA-2, q, S2, TI, (syn=a), (syn=P28), (syn=T-I), (syn=Tx), T3C, T5, (syn=T-5), (syn=T5), UC-I, w, β4, γ2, λ (syn=lambda), (syn=Φλ), ΦD326, φγ, Φ06, 0Φ7, Φ10, φ80, χ, (syn=χi), (syn=φχ), (syn=φχi), 2, 4, 4A, 6, 8A, 102, 150, 168, 174, 3000, AC6, AC7, AC28, AC43, AC50, AC57, AC81, AC95, H K243, KIO, ZG/3A, 5, 5A, 21EL, H19-J and 933H.

Bacteria of the genus Fusobacterium can be infected by the following phages: NN-Fusobacterium (2), fv83-554/3, fv88-531/2, 227, fv2377, fv2527 and fv8501.

Bacteria of the genus Haemophilus can be infected by the following phages: HPI, S2 and N3.

Bacteria of the genus Helicobacter can be infected by the following phages: HPI and \hat{ }\hat{ }-Helicobacter (1).

Bacteria of the genus Klebsiella can be infected by the following phages: AIO-2, KI4B, KI6B, KI9, (syn=K19), KI14, KI15, KI21, KI28, KI29, KI32, KI33, KI35, KI106B, KI171B, KI181B, KI832B, AIO-I, AO-I, AO-2, AO-3, FC3-10, K, KI1, (syn=KII), KI2, (syn=K12), KI3, (syn=K13), (syn=KI 70/11), KI4, (syn=K14), KI5, (syn=K15), KI6, (syn=K16), KI7, (syn=K17), KI8, (syn=KI8), KI19, (syn=K19), KI27, (syn=K127), KI31, (syn=K131), KI35, KI171B, II, VI, IX, CI-I, KI4B, KI8, KI11, KI12, KI13, KI16, KI17, KI18, KI20, KI22, KI23, KI24, KI26, KI30, KI34, KI106B, KIi65B, KI328B, KLXI, K328, P5046, 11, 380, III, IV, VII, VIII, FC3-11, KI2B, (syn=K12B), K125, (syn=K125), K142B, (syn=K142), (syn=K142B), K1181B, (syn=KII 81), (syn=K1181B), K1765/!, (syn=K1765/1), KI842B, (syn=K1832B), KI937B, (syn=K1937B), LI, φ28, 7, 231, 483, 490, 632 and 864/100.

Bacteria of the genus Lepitospira can be infected by the following phages: LEI, LE3, LE4 and ˜NN-Leptospira (1).

Bacteria of the genus Listeria can be infected by the following phages: A511, 01761, 4211, 4286, (syn=BO54), A005, A006, A020, A500, A502, A511, AI 18, A620, A640, B012, B021, B024, B025, B035, B051, B053, B054, B055, B056, BIOI, BI IO, B545, B604, B653, C707, D441, HSO47, HIOG, H8/73, H19, H21, H43, H46, H107, H108, HI IO, H163/84, H312, H340, H387, H391/73, H684/74, H924A, PSA, U153, φMLUP5, (syn=P35), 00241, 00611, 02971A, 02971C, 5/476, 5/911, 5/939, 5/11302, 5/11605, 5/11704, 184, 575, 633, 699/694, 744, 900, 1090, 1317, 1444, 1652, 1806, 1807, 1921/959, 1921/11367, 1921/11500, 1921/11566, 1921/12460, 1921/12582, 1967, 2389, 2425, 2671, 2685, 3274, 3550, 3551, 3552, 4276, 4277, 4292, 4477, 5337, 5348/11363, 5348/11646, 5348/12430, 5348/12434, 10072, 11355C, 11711A, 12029, 12981, 13441, 90666, 90816, 93253, 907515, 910716 and NN-Listeria (15).

Bacteria of the genus Morganella can be infected by the following phage: 47.

Bacteria of the genus Mycobacterium can be infected by the following phages: 13, AGI, ALi, ATCC 11759, A2, B.C3, BG2, BKI, BK5, butyricum, B-I, B5, B7, B30, B35, Clark, CI, C2, DNAIII, DSP1, D4, D29, GS4E, (syn=GS4E), GS7, (syn=GS-7), (syn=GS7), Pa, lacticola, Legendre, Leo, L5, (syn=ΦL-5), MC-I, MC-3, MC-4, minetti, MTPHI I, Mx4, MyF3P/59a, phlei, (syn=phlei 1), phlei 4, Polonus II, rabinovitschi, smegmatis, TM4, TM9, TMIO, TM20, Y7, YIO, φ630, IB, IF, IH, 1/1, 67, 106, 1430, BI, (syn=Bol), B24, D, D29, F-K, F-S, HP, Polonus I, Roy, RI, (syn=RI-Myb), (syn=Ri), 11, 31, 40, 50, 103a, 103b, 128, 3111-D, 3215-D and NN-Mycobacterium (1).

Bacteria of the genus Neisseria can be infected by the following phages: Group I, group II and NPI.

Bacteria of the genus Nocardia can be infected by the following phages: MNP8, NJ-L, NS-8, N5 and TtiN-Nocardia.

Bacteria of the genus Proteus can be infected by the following phages: Pm5, 13vir, 2/44, 4/545, 6/1004, 13/807, 20/826, 57, 67b, 78, 107/69, 121, 9/0, 22/608, 30/680, PmI, Pm3, Pm4, Pm6, Pm7, Pm9, PmIO, PmI I, Pv2, πI, φm, 7/549, 9B/2, 10A/31, 12/55, 14, 15, 16/789, 17/971, 19A/653, 23/532, 25/909, 26/219, 27/953, 32A/909, 33/971, 34/13, 65, 5006M, 7480b, VI, 13/3a, Clichy 12, π2600, φχ7, 1/1004, 5/742, 9, 12, 14, 22, 24/860, 2600/D52, Pm8 and 24/2514.

Bacteria of the genus Providencia can be infected by the following phages: PL25, PL26, PL37, 9211/9295, 9213/921 Ib, 9248, 7/R49, 7476/322, 7478/325, 7479, 7480, 9000/9402 and 9213/921 Ia.

Bacteria of the genus Pseudomonas can be infected by the following phages: Pfl, (syn=Pf-I), Pf2, Pf3, PP7, PRRI, 7s, im-Pseudomonas (1), AI-I, AI-2, B 17, B89, CB3, Col 2, Col 11, Col 18, Col 21, C154, C163, C167, C2121, E79, F8, ga, gb, H22, K1, M4, N2, Nu, PB-I, (syn=PBI), pfl6, PMN17, PPI, PP8, PsaI, PsPI, PsP2, PsP3, PsP4, PsP5, PS3, PS17, PTB80, PX4, PX7, PYOI, PYO2, PYO5, PYO6, PYO9, PYOIO, PYO13, PYO14, PYO16, PYO18, PYO19, PYO20, PYO29, PYO32, PYO33, PYO35, PYO36, PYO37, PYO38, PYO39, PYO41, PYO42, PYO45, PYO47, PYO48, PYO64, PYO69, PYO103, PIK, SLPI, SL2, S2, UNL-I, wy, Yai, Ya4, Yan, φBE, φCTX, φC17, φKZ, (syn=ΦKZ), T-LT, Φmu78, φNZ, φPLS-1, φST-1, φW-14, φ-2, 1/72, 2/79, 3, 3/DO, 4/237, 5/406, 6C, 6/6660, 7, 7v, 7/184, 8/280, 9/95, 10/502, 11/DE, 12/100, 12S, 16, 21, 24, 25F, 27, 31, 44, 68, 71, 95, 109, 188, 337, 352, 1214, HN-Pseudomonas (23), A856, B26, CI-I, CI-2, C5, D, gh-1, FI 16, HF, H90, K5, K6, KI 04, K109, K166, K267, N4, N5, 06N-25P, PE69, Pf, PPN25, PPN35, PPN89, PPN91, PP2, PP3, PP4, PP6, PP7, PP8, PP56, PP87, PPI 14, PP206, PP207, PP306, PP651, Psp231a, Pssy401, Pssy9220, psi, PTB2, PTB20, PTB42, PXI, PX3, PXIO, PX12, PX14, PYO70, PYO71, R, SH6, SH133, tf, Ya5, Ya7, φBS, ΦKf77, φ-MC, ΦmnF82, φPLS27, φPLS743, φS-1, 1, 2, 2, 3, 4, 5, 6, 7, 7, 8, 9, 10, 11, 12, 12B, 13, 14, 15, 14, 15, 16, 17, 18, 19, 20, 20, 21, 21, 22, 23, 23, 24, 25, 31, 53, 73, 119x, 145, 147, 170, 267, 284, 308, 525, NN-Pseudomonas (5), af, A7, B3, B33, B39, BI-1, C22, D3, D37, D40, D62, D3112, F7, FIO, g, gd, ge, gξ Hwl2, Jb 19, KFI, L°, OXN-32P, O6N-52P, PCH-1, PC13-1, PC35-1, PH2, PH51, PH93, PH132, PMW, PM13, PM57, PM61, PM62, PM63, PM69, PM105, PMI 13, PM681, PM682, P04, PPI, PP4, PP5, PP64, PP65, PP66, PP71, PP86, PP88, PP92, PP401, PP711, PP891, Pssy41, Pssy42, Pssy403, Pssy404, Pssy420, Pssy923, PS4, PS-10, Pz, SDI, SLI, SL3, SL5, SM, φC5, φCI I, φCI I-1, φC13, φC15, φMO, φX, φO4, φI I, φ240, 2, 2F, 5, 7m, 11, 13, 13/441, 14, 20, 24, 40, 45, 49, 61, 73, 148, 160, 198, 218, 222, 236, 242, 246, 249, 258, 269, 295, 297, 309, 318, 342, 350, 351, 357-1, 400-1, HN-Pseudomonas (6), GIOI, M6, M6a, LI, PB2, Pssyl5, Pssy4210, Pssy4220, PYO12, PYO34, PYO49, PYO50, PYO51, PYO52, PYO53, PYO57, PYO59, PYO200, PX2, PX5, SL4, φO3, φO6 and 1214.

Bacteria of the genus Rickettsia can be infected by the following phage: NN-Rickettsia.

Bacteria of the genus Salmonella can be infected by the following phages: b, Beccles, CT, d, Dundee, f, Fels 2, GI, GUI, GVI, GVIII, k, K, i, j, L, 01, (syn=0-1), (syn=O1), (syn=O-I), (syn=7), 02, 03, P3, P9a, PIO, Sab3, Sab5, SanIS, SanI7, SI, Taunton, Vil, (syn=Vil), 9, imSalmonella (1), N-I, N-5, N-IO, N-17, N-22, 11, 12, 16-19, 20.2, 36, 449C/C178, 966A/C259, a, B.A.O.R., e, G4, GUI, L, LP7, M, MG40, N-18, PSA68, P4, P9c, P22, (syn=P22), (syn=PLT22), (syn=PLT22), P22aI, P22-4, P22-7, P22-11, SNT-I, SNT-2, SP6, ViIIi, ViIV, ViV, ViVI, ViVII, Worksop, Sj5, ε34, 1,37, 1(40), (syn=φI[40]), 1,422, 2, 2.5, 3b, 4, 5, 6,14(18), 8, 14(6,7), 10, 27, 28B, 30, 31, 32, 33, 34, 36, 37, 39, 1412, SNT-3, 7-11, 40.3, c, C236, C557, C625, C966N, g, GV, G5, GI 73, h, IRA, Jersey, MB78, P22-1, P22-3, P22-12, SabI, Sab2, Sab2, Sab4, SanI, San2, San3, San4, San6, San7, San8, San9, SanI3, SanI4, SanI6, SanI8, SanI9, San20, San21, San22, San23, San24, San25, San26, SasLI, SasL2, SasL3, SasL4, SasL5, SIBL, SII, ViII, φI, 1, 2, 3a, 3al, 1010, Ym-Salmonella (1), N-4, SasL6 and 27.

Bacteria of the genus Serratia can be infected by the following phages: A2P, PS20, SMB3, SMP, SMP5, SM2, V40, V56, ic, ΦCP-3, ΦCP-6, 3M, 10/Ia, 20A, 34CC, 34H, 38T, 345G, 345P, 501B, SMB2, SMP2, BC, BT, CW2, CW3, CW4, CW5, Lt232, L2232, L34, L.228, SLP, SMPA, V.43, σ, ΦCWI, ΦCP6-1, ΦCP6-2, ΦCP6-5, 3T, 5, 8, 9F, 10/1, 20E, 32/6, 34B, 34CT, 34P, 37, 41, 56, 56D, 56P, 60P, 61/6, 74/6, 76/4, 101/8900, 226, 227, 228, 229F, 286, 289, 290F, 512, 764a, 2847/10, 2847/1Oa, L.359 and SMBI.

Bacteria of the genus Shigella can be infected by the following phages: Fsa, (syn=a), FSD2d, (syn=D2d), (syn=W2d), FSD2E, (syn=W2e), fv, F6, f7.8, H-Sh, PE5, P90, SflI, Sh, SHm, SHrv, (syn=HIV), SHvi, (syn=HVI), SHVvm, (syn=HVIII), SKy66, (syn=gamma 66), (syn=yββ), (syn=γ66b), SKm, (syn=SIIIb)5 (syn=UI), SKw, (syn=Siva), (syn=IV), SIC™, (syn=SIVA.), (syn=IVA), SKvi, (syn=KVI), (syn=Svi), (syn=VI), SKvm, (syn=Svm), (syn=VIII), SKVfI-A, (syn=SvmA), (syn=VIIIA), STvi, STK, STx1, STxn, S66, W2, (syn=D2c), (syn=D20), φI, φIVb 3-SO-R, 8368-SO-R, F7, (syn=FS7), (syn=K29), FIO, (syn=FSIO), (syn=K31), 11, (syn=alfa), (syn=FSa), (syn=KI 8), (syn=a), 12, (syn=a), (syn=K19), SG33, (syn=G35), (syn=SO-35/G), SG35, (syn=SO-55/G), SG3201, (syn=SO-3201/G), SHn, (syn=HII), SHv, (syn=SHV), SHx, SHX, SKn, (syn=K2), (syn=KII), (syn=Sn), (syn=SsII), (syn=II), SKrv, (syn=Sm), (syn=SslV), (syn=IV), SK1Va, (syn=Swab), (syn=SsIVa), (syn=IVa), SKV, (syn=K4), (syn=KV), (syn=SV), (syn=SsV), (syn=V), SKx, (syn=K9), (syn=KX), (syn=SX), (syn=SsX), (syn=X), STV, (syn=T35), (syn=35-50-R), STvm, (syn=T8345), (syn=8345-SO-S-R), W1, (syn=D8), (syn=FSD8), W2a, (syn=D2A), (syn=FS2a), DD-2, Sf6, FSi, (syn=FI), SF6, (syn=F6), SG42, (syn=SO-42/G), SG3203, (syn=SO-3203/G), SKF12, (syn=SsF12), (syn=F12), (syn=F12), STn, (syn=1881-SO-R), γ66, (syn=gamma 66a), (syn=Ssγ66), P2, BII, DDVII, (syn=DD7), FSD2b, (syn=W2B), FS2, (syn=F2), (syn=F2), FS4, (syn=F4), (syn=F4), FS5, (syn=F5), (syn=F5), FS9, (syn=F9), (syn=F9), FI I, P2-SO-S, SG36, (syn=SO-36/G), (syn=G36), SG3204, (syn=SO-3204/G), SG3244, (syn=SO-3244/G), SHi, (syn=HI), SHvπ, (syn=HVII), SHK, (syn=HIX), SHx1, SHxπ, (syn=HXn), SKI, KI, (syn=S1), (syn=Ssl), SKVII, (syn=KVII), (syn=Svπ), (syn=SsVII), SKIX, (syn=KIX), (syn=S1x), (syn=SslX), SKXII, (syn=KXII), (syn=Sxn), (syn=SsXII), STi, STffl, STrv, STVi, STvπ, S70, S206, U2-SO-S, 3210-SO-S, 3859-SO-S, 4020-SO-S, φ3, φ5, φ7, φ8, φ9, φIO, φI I, φI3, φI4, φI8, SHm, (syn=Hπi), SHχi, (syn=HXt) and SKxI, (syn=KXI), (syn=Sχi), (syn=SsXI), (syn=XI).

Bacteria of the genus Staphylococcus can be infected by the following phages: A, EW, K, Ph5, Ph9, PhIO, PhI3, PI, P2, P3, P4, P8, P9, PIO, RG, SB-i, (syn=Sb-I), S3K, Twort, ΦSK311, φ812, 06, 40, 58, 119, 130, 131, 200, 1623, STCI, (syn=stcl), STC2, (syn=stc2), 44AHJD, 68, ACI, AC2, A6″C″, A9″C, b581, CA-I, CA-2, CA-3, CA-4, CA-5, DI I, L39x35, L54a, M42, NI, N2, N3, N4, N5, N7, N8, NIO, Ni I, N12, N13, N14, N16, Ph6, PhI2, PhI4, UC-18, U4, U15, SI, S2, S3, S4, S5, X2, Z1, φB5-2, φD, ω, 11, (syn=φI I), (syn=P11-M15), 15, 28, 28A, 29, 31, 31B, 37, 42D, (syn=P42D), 44A, 48, 51, 52, 52A, (syn=P52A), 52B, 53, 55, 69, 71, (syn=P71), 71A, 72, 75, 76, 77, 79, 80, 80a, 82, 82A, 83 A, 84, 85, 86, 88, 88A, 89, 90, 92, 95, 96, 102, 107, 108, 111, 129-26, 130, 130A, 155, 157, 157A, 165, 187, 275, 275A, 275B, 356, 456, 459, 471, 471A, 489, 581, 676, 898, 1139, 1154A, 1259, 1314, 1380, 1405, 1563, 2148, 2638A, 2638B, 2638C, 2731, 2792A, 2792B, 2818, 2835, 2848A, 3619, 5841, 12100, AC3, A8, AIO, A13, b594n, D, HK2, N9, N15, P52, P87, SI, S6, Z4, φRE, 3A, 3B, 3C, 6, 7, 16, 21, 42B, 42C, 42E, 44, 47, 47A5 47C, 51, 54, 54x1, 70, 73, 75, 78, 81, 82, 88, 93, 94, 101, 105, 110, 115, 129/16, 174, 594n, 1363/14, 2460 and mS-Staphylococcus (1).

Bacteria of the genus Streptococcus can be infected by the following phages: EJ-1, NN-Streptococais (1), a, CI, FL0Ths, H39, Cp-I, Cp-5, Cp-7, Cp-9, Cp-IO, AT298, A5, aIO/JI, aIO/J2, aIO/J5, aIO/J9, A25, BTI I, b6, CAI, c20-1, c20-2, DP-I, Dp-4, DTI, ET42, eIO, FA101, FEThs, F_{K}, FKKIOI, FKLIO, FKP74, FKH, FLOThs, FyIOI, fl, F10, F20140/76, g, GT-234, HB3, (syn=HB-3), HB-623, HB-746, M102, O1205, φO1205, PST, PO, PI, P2, P3, P5, P6, P8, P9, P9, P12, P13, P14, P49, P50, P51, P52, P53, P54, P55, P56, P57, P58, P59, P64, P67, P69, P71, P73, P75, P76, P77, P82, P83, P88, sc, sch, sf, SflI 1, (syn=SFil I), (syn=φSFilI), (syn=CDSfil I), (syn=φSfil I), sfiI9, (syn=SFiI9), (syn=φSFiI9), (syn=pSfiI9), Sfi21, (syn=SFi21), (syn=φSFi21), (syn=φSfi21), ST0, STX, st2, ST2, ST4, S3, (syn=φS3), s265, Φ17, φ42, φ57, φ80, φ81, φ82, φ83, φ84, φ85, φ86, φ87, φ88, φ89, φ90, φ91, φ92, φ93, φ94, φ95, φ96, φ97, φ98, φ99, φIOO, φIOI, φIO2, φ227, Φ7201, ω1, ω2, ω3, ω4, ω5, ω6, ω8, ωIO, 1, 6, 9, 1OF, 12/12, 14, 17SR, 19S, 24, 50/33, 50/34, 55/14, 55/15, 70/35, 70/36, 71/ST15, 71/45, 71/46, 74F, 79/37, 79/38, 80/J4, 80/J9, 80/ST16, 80/15, 80/47, 80/48, 101, 103/39, 103/40, 121/41, 121/42, 123/43, 123/44, 124/44, 337/ST17 and mStreptococcus (34).

Bacteria of the genus Treponema can be infected by the following phage: NN-Treponema (1).

Bacteria of the genus Vibrio can be infected by the following phages: CTXΦ, fs, (syn=si), fs2, Ivpf5, Vfl2, Vf33, VPIΦ, VSK, v6, 493, CP-TI, ET25, kappa, K139, Labol, )XN-69P, OXN-86, O6N-21P, PB-I, P147, rp-1, SE3, VA-1, (syn=VcA-1), VcA-2, VPI, VP2, VP4, VP7, VP8, VP9, VPIO, VP17, VP18, VP19, X29, (syn=29 d'Herelle), t, ΦHAWI-1, ΦHAWI-2, ΦHAWI-3, ΦHAWI-4, ΦHAWI-5, ΦHAWI-6, ΦHAWI-7, XHAWI-8, ΦHAWI-9, ΦHAWI-10, ΦHCl-1, ΦHCl-2, ΦHCl-3, ΦHC1-4, ΦHC2-1, >HC2-2, ΦHC2-3, ΦHC2-4, ΦHC3-1, ΦHC3-2, ΦHC3-3, ΦHD1S-1, ΦHD1S-2, ΦHD2S-1, ΦHD2S-2, ΦHD2S-3, ΦHD2S-4, ΦHD2S-5, ΦHDO-1, ΦHDO-2, ΦHDO-3, ΦHDO-4, ΦHDO-5, ΦHDO-6, ΦKL-33, ΦKL-34, ΦKL-35, ΦKL-36, ΦKWH-2, ΦKWH-3, ΦKWH-4, ΦMARQ-1, ΦMARQ-2, ΦMARQ-3, ΦMOAT-1, ΦO139, ΦPEL1A-1, ΦPEL1A-2, ΦPEL8A-1, ΦPEL8A-2, ΦPEL8A-3, ΦPEL8C-1, ΦPEL8C-2, ΦPEL13A-1, ΦPEL13B-1, ΦPEL13B-2, ΦPEL13B-3, ΦPEL13B-4, ΦPEL13B-5, ΦPEL13B-6, ΦPEL13B-7, ΦPEL13B-8, ΦPEL13B-9, ΦPEL13B-10, φVP143, φVP253, Φ16, φI38, 1-II, 5, 13, 14, 16, 24, 32, 493, 6214, 7050, 7227, II, (syn=group II), (syn==p2), V, VIII, ˜m-Vibrio (13), KVP20, KVP40, nt-1, 06N-22P, P68, el, e2, e3, e4, e5, FK, G, I, K, nt-6, NI, N2, N3, N4, N5, O6N-34P, OXN-72P, OXN-85P, OXN-100P, P, Ph-I, PL163/10, Q, S, T, φ92, 1-9, 37, 51, 57, 70A-8, 72A-4, 72A-10, 110A-4, 333, 4996, 1 (syn=group 1), III (syn=group III), VI, (syn=A-Saratov), VII, IX, X, HN-Vibrio (6), pAI, 7, 7-8, 70A-2, 71A-6, 72A-5, 72A-8, 108A-10, 109A-6, 109A-8, IIOA-1, 110A-5, 110A-7, hv-1, OXN-52P, P13, P38, P53, P65, P108, PiII, TP13 VP3, VP6, VP12, VP13, 70A-3, 70A-4, 70A-10, 72A-1, 108A-3, 109-B1, 110A-2, 149, (syn=p149), IV, (syn=group IV), NN-Vibrio (22), VP5, VPII, VP15, VP16, αI, α2, α3a, α3b, 353B and HN-Vibrio (7).

Bacteria of the genus Yersinia can be infected by the following phages: H, H-1, H-2, H-3, H-4, Lucas 110, Lucas 303, Lucas 404, YerA3, YerA7, YerA20, YerA41, 3/M64-76, 5/G394-76, 6/C753-76, 8/C239-76, 9/F18167, 1701, 1710, PST, 1/F2852-76, D'Herelle, EV, H, Kotljarova, PTB, R, Y, YerA41, φYerO3-12, 3, 4/C1324-76, 7/F783-76, 903, 1/M6176 and Yer2AT.

In an embodiment, the bacteriophage is selected in the group consisting of Salmonella virus SKML39, Shigella virus AG3, Dickeya virus Limestone, Dickeya virus RC2014, Escherichia virus CBA120, Escherichia virus PhaxI, Salmonella virus 38, Salmonella virus Det7, Salmonella virus GG32, Salmonella virus PM10, Salmonella virus SFP10, Salmonella virus SH19, Salmonella virus SJ3, Escherichia virus ECML4, Salmonella virus Marshall, Salmonella virus Maynard, Salmonella virus SJ2, Salmonella virus STML131, Salmonella virus Vil, Erwinia virus Ea2809, Klebsiella virus 0507KN21, Serratia virus IME250, Serratia virus MAM1, Campylobacter virus CP21, Campylobacter virus CP220, Campylobacter virus CPt10, Campylobacter virus IBB35, Campylobacter virus CP81, Campylobacter virus CP30A, Campylobacter virus CPX, Campylobacter virus NCTC12673, Erwinia virus Ea214, Erwinia virus M7, Escherichia virus AYO145A, Escherichia virus EC6, Escherichia virus HY02, Escherichia virus JH2, Escherichia virus TP1, Escherichia virus VpaE1, Escherichia virus wV8, Salmonella virus Felix01, Salmonella virus HB2014, Salmonella virus Mushroom, Salmonella virus UAB87, Citrobacter virus Moogle, Citrobacter virus Mordin, Escherichia virus SUSP1, Escherichia virus SUSP2, Aeromonas virus phiO18P, Haemophilus virus HP1, Haemophilus virus HP2, Pasteurella virus F108, Vibrio virus K139, Vibrio virus Kappa, Burkholderia virus phi52237, Burkholderia virus phiE122, Burkholderia virus phiE202, Escherichia virus 186, Escherichia virus P4, Escherichia virus P2, Escherichia virus Wphi, Mannheimia virus PHL101, Pseudomonas virus phiCTX, Ralstonia virus RSA1, Salmonella virus Fels2, Salmonella virus PsP3, Salmonella virus SopEphi, Yersinia virus L413C, Staphylococcus virus G1, Staphylococcus virus G15, Staphylococcus virus JD7, Staphylococcus virus K, Staphylococcus virus MCE2014, Staphylococcus virus P108, Staphylococcus virus Rodi, Staphylococcus virus S253, Staphylococcus virus S25-4, Staphylococcus virus SA12, Listeria virus A511, Listeria virus P100, Staphylococcus virus Remus, Staphylococcus virus SA11, Staphylococcus virus Stau2, Bacillus virus Camphawk, Bacillus virus SPO1, Bacillus virus BCP78, Bacillus virus TsarBomba, Staphylococcus virus Twort, Enterococcus virus phiEC24C, Lactobacillus virus Lb338-1, Lactobacillus virus LP65, Enterobacter virus PG7, Escherichia virus CC31, Klebsiella virus JD18, Klebsiella virus PKO111, Escherichia virus Bp7, Escherichia virus IME08, Escherichia virus JS10, Escherichia virus JS98, Escherichia virus QL01, Escherichia virus VR5, Enterobacter virus Eap3, Klebsiella virus KP15, Klebsiella virus KP27, Klebsiella virus Matisse, Klebsiella virus Miro, Citrobacter virus Merlin, Citrobacter virus Moon, Escherichia virus JSE, Escherichia virus phi1, Escherichia virus RB49, Escherichia virus HX01, Escherichia virus JS09, Escherichia virus RB69, Shigella virus UTAM, Salmonella virus S16, Salmonella virus STML198, Vibrio virus KVP40, Vibrio virus nt1, Vibrio virus ValKK3, Escherichia virus VR7, Escherichia virus VR20, Escherichia virus VR25, Escherichia virus VR26, Shigella virus SP18, Escherichia virus AR1, Escherichia virus C40, Escherichia virus E112, Escherichia virus ECML134, Escherichia virus HY01, Escherichia virus Ime09, Escherichia virus RB3, Escherichia virus RB14, Escherichia virus T4, Shigella virus Pss1, Shigella virus Shfl2, Yersinia virus D1, Yersinia virus PST, Acinetobacter virus 133, Aeromonas virus 65, Aeromonas virus Aeh1, Escherichia virus RB16, Escherichia virus RB32, Escherichia virus RB43, Pseudomonas virus 42, Cronobacter virus CR3, Cronobacter virus CR8, Cronobacter virus CR9, Cronobacter virus PBES02, Pectobacterium virus phiTE, Cronobacter virus GAP31, Escherichia virus 4MG, Salmonella virus SE1, Salmonella virus SSE121, Escherichia virus FFH2, Escherichia virus FV3, Escherichia virus JES2013, Escherichia virus V5, Brevibacillus virus Abouo, Brevibacillus virus Davies, Bacillus virus Agate, Bacillus virus Bobb, Bacillus virus Bp8pC, Erwinia virus Deimos, Erwinia virus Ea35-70, Erwinia virus RAY, Erwinia virus Simmy50, Erwinia virus SpecialG, Acinetobacter virus AB1, Acinetobacter virus AB2, Acinetobacter virus AbC62, Acinetobacter virus AP22, Arthrobacter virus ArV1, Arthrobacter virus Trina, Bacillus virus AvesoBmore, Bacillus virus B4, Bacillus virus Bigbertha, Bacillus virus Riley, Bacillus virus Spock, Bacillus virus Troll, Bacillus virus Bastille, Bacillus virus CAM003, Bacillus virus Bc431, Bacillus virus Bcp1, Bacillus virus BCP82, Bacillus virus BM15, Bacillus virus Deepblue, Bacillus virus JBP901, Burkholderia virus Bcep1, Burkholderia virus Bcep43, Burkholderia virus Bcep781, Burkholderia virus BcepNY3, Xanthomonas virus OP2, Burkholderia virus BcepMu, Burkholderia virus phiE255, Aeromonas virus 44RR2, Mycobacterium virus Alice, Mycobacterium virus Bxz1, Mycobacterium virus Dandelion, Mycobacterium virus HyRo, Mycobacterium virus 13, Mycobacterium virus Nappy, Mycobacterium virus Sebata, Clostridium virus phiC2, Clostridium virus phiCD27, Clostridium virus phiCD119, Bacillus virus CP51, Bacillus virus JL, Bacillus virus Shanette, Escherichia virus CVM10, Escherichia virus ep3, Erwinia virus Asesino, Erwinia virus EaH2, Pseudomonas virus EL, Halomonas virus HAP1, Vibrio virus VP882, Brevibacillus virus Jimmer, Brevibacillus virus Osiris, Pseudomonas virus Ab03, Pseudomonas virus KPP10, Pseudomonas virus PAKP3, Sinorhizobium virus M7, Sinorhizobium virus M12, Sinorhizobium virus N3, Erwinia virus Machina, Arthrobacter virus Brent, Arthrobacter virus Jawnski, Arthrobacter virus Martha, Arthrobacter virus Sonny, Edwardsiella virus MSW3, Edwardsiella virus PEi21, Escherichia virus Mu, Shigella virus SfMu, Halobacterium virus phiH, Bacillus virus Grass, Bacillus virus NIT1, Bacillus virus SPG24, Aeromonas virus 43, Escherichia virus P1, Pseudomonas virus CAb1, Pseudomonas virus CAb02, Pseudomonas virus JG004, Pseudomonas virus PAKP1, Pseudomonas virus PAKP4, Pseudomonas virus PaP1, Burkholderia virus BcepF1, Pseudomonas virus 141, Pseudomonas virus Ab28, Pseudomonas virus DL60, Pseudomonas virus DL68, Pseudomonas virus F8, Pseudomonas virus JG024, Pseudomonas virus KPP12, Pseudomonas virus LBL3, Pseudomonas virus LMA2, Pseudomonas virus PB1, Pseudomonas virus SN, Pseudomonas virus PA7, Pseudomonas virus phiKZ, Rhizobium virus RHEph4, Ralstonia virus RSF1, Ralstonia virus RSL2, Ralstonia virus RSL1, Aeromonas virus 25, Aeromonas virus 31, Aeromonas virus Aes12, Aeromonas virus Aes508, Aeromonas virus AS4, Stenotrophomonas virus IME13, Staphylococcus virus IPLAC1C, Staphylococcus virus SEP1, Salmonella virus SPN3US, Bacillus virus 1, Geobacillus virus GBSV1, Yersinia virus R1RT, Yersinia virus TG1, Bacillus virus G, Bacillus virus PBS1, Microcystis virus Ma-LMM01, Vibrio virus MAR, Vibrio virus VHML, Vibrio virus VP585, Bacillus virus BPS13, Bacillus virus Hakuna, Bacillus virus Megatron, Bacillus virus WPh, Acinetobacter virus AB3, Acinetobacter virus Abp1, Acinetobacter virus Fri1, Acinetobacter virus IME200, Acinetobacter virus PD6A3, Acinetobacter virus PDAB9, Acinetobacter virus phiAB1, Escherichia virus K30, Klebsiella virus K5, Klebsiella virus K11, Klebsiella virus Kp1, Klebsiella virus KP32, Klebsiella virus KpV289, Klebsiella virus F19, Klebsiella virus K244, Klebsiella virus Kp2, Klebsiella virus KP34, Klebsiella virus KpV41, Klebsiella virus KpV71, Klebsiella virus KpV475, Klebsiella virus SU503, Klebsiella virus SU552A, Pantoea virus Limelight, Pantoea virus Limezero, Pseudomonas virus LKA1, Pseudomonas virus phiKMV, Xanthomonas virus f20, Xanthomonas virus f30, Xylella virus Prado, Erwinia virus Eral03, Escherichia virus K5, Escherichia virus K1-5, Escherichia virus K1E, Salmonella virus SP6, Escherichia virus T7, Kluyvera virus Kvp1, Pseudomonas virus gh1, Prochlorococcus virus PSSP7, Synechococcus virus P60, Synechococcus virus Syn5, Streptococcus virus Cp1, Streptococcus virus Cp7, Staphylococcus virus 44AHJD, Streptococcus virus C1, Bacillus virus B103, Bacillus virus GA1, Bacillus virus phi29, Kurthia virus 6, Actinomyces virus Av1, Mycoplasma virus P1, Escherichia virus 24B, Escherichia virus 933W, Escherichia virus Min27, Escherichia virus PA28, Escherichia virus Stx2 II, Shigella virus 7502Stx, Shigella virus POCJ13, Escherichia virus 191, Escherichia virus PA2, Escherichia virus TL2011, Shigella virus VASD, Burkholderia virus Bcep22, Burkholderia virus Bcepil02, Burkholderia virus Bcepmigl, Burkholderia virus DC1, Bordetella virus BPP1, Burkholderia virus BcepC6B, Cellulophaga virus Cba41, Cellulophaga virus Cba172, Dinoroseobacter virus DFL12, Erwinia virus Ea9-2, Erwinia virus Frozen, Escherichia virus phiV10, Salmonella virus Epsilon15, Salmonella virus SPN1S, Pseudomonas virus F116, Pseudomonas virus H66, Escherichia virus APEC5, Escherichia virus APEC7, Escherichia virus Bp4, Escherichia virus EC1UPM, Escherichia virus ECBP1, Escherichia virus G7C, Escherichia virus IME11, Shigella virus Sb1, Achromobacter virus Axp3, Achromobacter virus JWAlpha, Edwardsiella virus KF1, Pseudomonas virus KPP25, Pseudomonas virus R18, Pseudomonas virus Ab09, Pseudomonas virus LIT1, Pseudomonas virus PA26, Pseudomonas virus Ab22, Pseudomonas virus CHU, Pseudomonas virus LUZ24, Pseudomonas virus PAA2, Pseudomonas virus PaP3, Pseudomonas virus PaP4, Pseudomonas virus TL, Pseudomonas virus KPP21, Pseudomonas virus LUZ7, Escherichia virus N4, Salmonella virus 9NA, Salmonella virus SP069, Salmonella virus BTP1, Salmonella virus HK620, Salmonella virus P22, Salmonella virus ST64T, Shigella virus Sf6, Bacillus virus Page, Bacillus virus Palmer, Bacillus virus Pascal, Bacillus virus Pony, Bacillus virus Pookie, Escherichia virus 172-1, Escherichia virus ECB2, Escherichia virus NJ01, Escherichia virus phiEco32, Escherichia virus Septima11, Escherichia virus SU10, Brucella virus Pr, Brucella virus Tb, Escherichia virus Pollock, Salmonella virus FSL SP-058, Salmonella virus FSL SP-076, Helicobacter virus 1961 P, Helicobacter virus KHP30, Helicobacter virus KHP40, Hamiltonella virus APSE1, Lactococcus virus KSY1, Phormidium virus WMP3, Phormidium virus WMP4, Pseudomonas virus 119X, Roseobacter virus SIO1, Vibrio virus VpV262, Vibrio virus VC8, Vibrio virus VP2, Vibrio virus VP5, Streptomyces virus Amela, Streptomyces virus phiCAM, Streptomyces virus Aaronocolus, Streptomyces virus Caliburn, Streptomyces virus Danzina, Streptomyces virus Hydra, Streptomyces virus Izzy, Streptomyces virus Lannister, Streptomyces virus Lika, Streptomyces virus Sujidade, Streptomyces virus Zemlya, Streptomyces virus ELB20, Streptomyces virus R4, Streptomyces virus phiHau3, Mycobacterium virus Acadian, Mycobacterium virus Baee, Mycobacterium virus Reprobate, Mycobacterium virus Adawi, Mycobacterium virus Bane1, Mycobacterium virus BrownCNA, Mycobacterium virus Chrisnmich, Mycobacterium virus Cooper, Mycobacterium virus JAMaL, Mycobacterium virus Nigel, Mycobacterium virus Stinger, Mycobacterium virus Vincenzo, Mycobacterium virus Zemanar, Mycobacterium virus Apizium, Mycobacterium virus Manad, Mycobacterium virus Oline, Mycobacterium virus Osmaximus, Mycobacterium virus Pg1, Mycobacterium virus Soto, Mycobacterium virus Suffolk, Mycobacterium virus Athena, Mycobacterium virus Bernardo, Mycobacterium virus Gadjet, Mycobacterium virus Pipefish, Mycobacterium virus Godines, Mycobacterium virus Rosebush, Mycobacterium virus Babsiella, Mycobacterium virus Brujita, Mycobacterium virus Che9c, Mycobacterium virus Sbash, Mycobacterium virus Hawkeye, Mycobacterium virus Plot, Salmonella virus AG11, Salmonella virus Ent1, Salmonella virus f18SE, Salmonella virus Jersey, Salmonella virus L13, Salmonella virus LSPA1, Salmonella virus SE2, Salmonella virus SETP3, Salmonella virus SETP7, Salmonella virus SETP13, Salmonella virus SP101, Salmonella virus SS3e, Salmonella virus wksl3, Escherichia virus K1G, Escherichia virus K1H, Escherichia virus K1ind1, Escherichia virus K1ind2, Salmonella virus SP31, Leuconostoc virus Lmd1, Leuconostoc virus LN03, Leuconostoc virus LN04, Leuconostoc virus LN12, Leuconostoc virus LN6B, Leuconostoc virus P793, Leuconostoc virus 1A4, Leuconostoc virus Ln8, Leuconostoc virus Ln9, Leuconostoc virus LN25, Leuconostoc virus LN34, Leuconostoc virus LNTR3, Mycobacterium virus Bongo, Mycobacterium virus Rey, Mycobacterium virus Butters, Mycobacterium virus Michelle, Mycobacterium virus Charlie, Mycobacterium virus Pipsqueaks, Mycobacterium virus Xeno, Mycobacterium virus Panchino, Mycobacterium virus Phrann, Mycobacterium virus Redi, Mycobacterium virus Skinnyp, Gordonia virus BaxterFox, Gordonia virus Yeezy, Gordonia virus Kita, Gordonia virus Zirinka, Gorrdonia virus Nymphadora, Mycobacterium virus Bignuz, Mycobacterium virus Brusacoram, Mycobacterium virus Donovan, Mycobacterium virus Fishburne, Mycobacterium virus Jebeks, Mycobacterium virus Malithi, Mycobacterium virus Phayonce, Enterobacter virus F20, Klebsiella virus 1513, Klebsiella virus KLPN1, Klebsiella virus KP36, Klebsiella virus PKP126, Klebsiella virus Sushi, Escherichia virus AHP42, Escherichia virus AHS24, Escherichia virus AKS96, Escherichia virus C119, Escherichia virus E41c, Escherichia virus Eb49, Escherichia virus Jk06, Escherichia virus KP26, Escherichia virus Rogue1, Escherichia virus ACGM12, Escherichia virus Rtp, Escherichia virus ADB2, Escherichia virus JMPW1, Escherichia virus JMPW2, Escherichia virus T1, Shigella virus PSf2, Shigella virus Shfl1, Citrobacter virus Stevie, Escherichia virus TLS, Salmonella virus SP126, Cronobacter virus Esp2949-1, Pseudomonas virus Ab18, Pseudomonas virus Ab19, Pseudomonas virus PaMx11, Arthrobacter virus Amigo, Propionibacterium virus Anatole, Propionibacterium virus B3, Bacillus virus Andromeda, Bacillus virus Blastoid, Bacillus virus Curly, Bacillus virus Eoghan, Bacillus virus Finn, Bacillus virus Glittering, Bacillus virus Riggi, Bacillus virus Taylor, Gordonia virus Attis, Mycobacterium virus Barnyard, Mycobacterium virus Konstantine, Mycobacterium virus Predator, Mycobacterium virus Bernal13, Staphylococcus virus 13, Staphylococcus virus 77, Staphylococcus virus 108PVL, Mycobacterium virus Bron, Mycobacterium virus Faith1, Mycobacterium virus Joedirt, Mycobacterium virus Rumpelstiltskin, Lactococcus virus blL67, Lactococcus virus c2, Lactobacillus virus c5, Lactobacillus virus Ld3, Lactobacillus virus Ld17, Lactobacillus virus Ld25A, Lactobacillus virus LLKu, Lactobacillus virus phiLdb, Cellulophaga virus Cba121, Cellulophaga virus Cba171, Cellulophaga virus Cba181, Cellulophaga virus ST, Bacillus virus 250, Bacillus virus IEBH, Mycobacterium virus Ardmore, Mycobacterium virus Avani, Mycobacterium virus Boomer, Mycobacterium virus Che8, Mycobacterium virus Che9d, Mycobacterium virus Deadp, Mycobacterium virus Dlane, Mycobacterium virus Dorothy, Mycobacterium virus Dotproduct, Mycobacterium virus Drago, Mycobacterium virus Fruitloop, Mycobacterium virus Gumbie, Mycobacterium virus Ibhubesi, Mycobacterium virus Llij, Mycobacterium virus Mozy, Mycobacterium virus Mutaforma13, Mycobacterium virus Pacc40, Mycobacterium virus PMC, Mycobacterium virus Ramsey, Mycobacterium virus Rockyhorror, Mycobacterium virus SG4, Mycobacterium virus Shauna1, Mycobacterium virus Shilan, Mycobacterium virus Spartacus, Mycobacterium virus Taj, Mycobacterium virus Tweety, Mycobacterium virus Wee, Mycobacterium virus Yoshi, Salmonella virus Chi, Salmonella virus FSLSPO30, Salmonella virus FSLSP088, Salmonella virus iEPS5, Salmonella virus SPN19, Mycobacterium virus 244, Mycobacterium virus Bask21, Mycobacterium virus CJW1, Mycobacterium virus Eureka, Mycobacterium virus Kostya, Mycobacterium virus Porky, Mycobacterium virus Pumpkin, Mycobacterium virus Sirduracell, Mycobacterium virus Toto, Mycobacterium virus Corndog, Mycobacterium virus Firecracker, Rhodobacter virus RcCronus, Pseudomonas virus D3112, Pseudomonas virus DMS3, Pseudomonas virus FHA0480, Pseudomonas virus LPB1, Pseudomonas virus MP22, Pseudomonas virus MP29, Pseudomonas virus MP38, Pseudomonas virus PA1KOR, Pseudomonas virus D3, Pseudomonas virus PMG1, Arthrobacter virus Decurro, Gordonia virus Demosthenes, Gordonia virus Katyusha, Gordonia virus Kvothe, Propionibacterium virus B22, Propionibacterium virus Doucette, Propionibacterium virus E6, Propionibacterium virus G4, Burkholderia virus phi6442, Burkholderia virus phi1026b, Burkholderia virus phiE125, Edwardsiella virus eiAU, Mycobacterium virus Ff47, Mycobacterium virus Muddy, Mycobacterium virus Gaia, Mycobacterium virus Giles, Arthrobacter virus Captnmurica, Arthrobacter virus Gordon, Gordonia virus GordTnk2, Paenibacillus virus Harrison, Escherichia virus EK99P1, Escherichia virus HK578, Escherichia virus JL1, Escherichia virus SSL2009a, Escherichia virus YD2008s, Shigella virus EP23, Sodalis virus SO1, Escherichia virus HK022, Escherichia virus HK75, Escherichia virus HK97, Escherichia virus HK106, Escherichia virus HK446, Escherichia virus HK542, Escherichia virus HK544, Escherichia virus HK633, Escherichia virus mEp234, Escherichia virus mEp235, Escherichia virus mEpX1, Escherichia virus mEpX2, Escherichia virus mEp043, Escherichia virus mEp213, Escherichia virus mEp237, Escherichia virus mEp390, Escherichia virus mEp460, Escherichia virus mEp505, Escherichia virus mEp506, Brevibacillus virus Jenst, Achromobacter virus 83-24, Achromobacter virus JWX, Arthrobacter virus Kellezzio, Arthrobacter virus Kitkat, Arthrobacter virus Bennie, Arthrobacter virus DrRobert, Arthrobacter virus Glenn, Arthrobacter virus HunterDalle, Arthrobacter virus Joann, Arthrobacter virus Korra, Arthrobacter virus Preamble, Arthrobacter virus Pumancara, Arthrobacter virus Wayne, Mycobacterium virus Alma, Mycobacterium virus Arturo, Mycobacterium virus Astro, Mycobacterium virus Backyardigan, Mycobacterium virus BBPiebs31, Mycobacterium virus Benedict, Mycobacterium virus Bethlehem, Mycobacterium virus Billknuckles, Mycobacterium virus Bruns, Mycobacterium virus Bxb1, Mycobacterium virus Bxz2, Mycobacterium virus Che12, Mycobacterium virus Cuco, Mycobacterium virus D29, Mycobacterium virus Doom, Mycobacterium virus Ericb, Mycobacterium virus Euphoria, Mycobacterium virus George, Mycobacterium virus Gladiator, Mycobacterium virus Goose, Mycobacterium virus Hammer, Mycobacterium virus Heldan, Mycobacterium virus Jasper, Mycobacterium virus JC27, Mycobacterium virus Jeffabunny, Mycobacterium virus JHC117, Mycobacterium virus KBG, Mycobacterium virus Kssjeb, Mycobacterium virus Kugel, Mycobacterium virus L5, Mycobacterium virus Lesedi, Mycobacterium virus LHTSCC, Mycobacterium virus lockley, Mycobacterium virus Marcell, Mycobacterium virus Microwolf, Mycobacterium virus Mrgordo, Mycobacterium virus Museum, Mycobacterium virus Nepal, Mycobacterium virus Packman, Mycobacterium virus Peaches, Mycobacterium virus Perseus, Mycobacterium virus Pukovnik, Mycobacterium virus Rebeuca, Mycobacterium virus Redrock, Mycobacterium virus Ridgecb, Mycobacterium virus Rockstar, Mycobacterium virus Saintus, Mycobacterium virus Skipole, Mycobacterium virus Solon, Mycobacterium virus Switzer, Mycobacterium virus SWU1, Mycobacterium virus Ta17a, Mycobacterium virus Tiger, Mycobacterium virus Timshel, Mycobacterium virus Trixie, Mycobacterium virus Turbido, Mycobacterium virus Twister, Mycobacterium virus U2, Mycobacterium virus Violet, Mycobacterium virus Wonder, Escherichia virus DE3, Escherichia virus HK629, Escherichia virus HK630, Escherichia virus lambda, Arthrobacter virus Laroye, Mycobacterium virus Halo, Mycobacterium virus Liefie, Mycobacterium virus Marvin, Mycobacterium virus Mosmoris, Arthrobacter virus Circum, Arthrobacter virus Mudcat, Escherichia virus N15, Escherichia virus 9g, Escherichia virus JenK1, Escherichia virus JenP1, Escherichia virus JenP2, Pseudomonas virus NP1, Pseudomonas virus PaMx25, Mycobacterium virus Baka, Mycobacterium virus Courthouse, Mycobacterium virus Littlee, Mycobacterium virus Omega, Mycobacterium virus Optimus, Mycobacterium virus Thibault, Polaribacter virus P12002L, Polaribacter virus P12002S, Nonlabens virus P12024L, Nonlabens virus P12024S, Thermus virus P23-45, Thermus virus P74-26, Listeria virus LP26, Listeria virus LP37, Listeria virus LP110, Listeria virus LP114, Listeria virus P70, Propionibacterium virus ATCC29399BC, Propionibacterium virus ATCC29399BT, Propionibacterium virus Attacne, Propionibacterium virus Keiki, Propionibacterium virus Kubed, Propionibacterium virus Lauchelly, Propionibacterium virus MrAK, Propionibacterium virus Ouroboros, Propionibacterium virus P91, Propionibacterium virus P105, Propionibacterium virus P144, Propionibacterium virus P1001, Propionibacterium virus P1.1, Propionibacterium virus P100A, Propionibacterium virus P100D, Propionibacterium virus P101A, Propionibacterium virus P104A, Propionibacterium virus PA6, Propionibacterium virus Pacnes201215, Propionibacterium virus PAD20, Propionibacterium virus PAS50, Propionibacterium virus PHL009M11, Propionibacterium virus PHL025M00, Propionibacterium virus PHL037M02, Propionibacterium virus PHL041M10, Propionibacterium virus PHL060L00, Propionibacterium virus PHL067M01, Propionibacterium virus PHL070N00, Propionibacterium virus PHL071N05, Propionibacterium virus PHL082M03, Propionibacterium virus PHL092M00, Propionibacterium virus PHL095N00, Propionibacterium virus PHL111M01, Propionibacterium virus PHL112N00, Propionibacterium virus PHL113M01, Propionibacterium virus PHL114L00, Propionibacterium virus PHL116M00, Propionibacterium virus PHL117M00, Propionibacterium virus PHL117M01, Propionibacterium virus PHL132N00, Propionibacterium virus PHL141N00, Propionibacterium virus PHL151M00, Propionibacterium virus PHL151 N00, Propionibacterium virus PHL152M00, Propionibacterium virus PHL163M00, Propionibacterium virus PHL171M01, Propionibacterium virus PHL179M00, Propionibacterium virus PHL194M00, Propionibacterium virus PHL199M00, Propionibacterium virus PHL301M00, Propionibacterium virus PHL308M00, Propionibacterium virus Pirate, Propionibacterium virus Procrass1, Propionibacterium virus SKKY, Propionibacterium virus Solid, Propionibacterium virus Stormborn, Propionibacterium virus Wizzo, Pseudomonas virus PaMx28, Pseudomonas virus PaMx74, Mycobacterium virus Patience, Mycobacterium virus PB11, Rhodococcus virus Pepy6, Rhodococcus virus Poco6, Propionibacterium virus PFR1, Streptomyces virus phiBT1, Streptomyces virus phiC31, Streptomyces virus TG1, Caulobacter virus Karma, Caulobacter virus Magneto, Caulobacter virus phiCbK, Caulobacter virus Rogue, Caulobacter virus Swift, Staphylococcus virus 11, Staphylococcus virus 29, Staphylococcus virus 37, Staphylococcus virus 53, Staphylococcus virus 55, Staphylococcus virus 69, Staphylococcus virus 71, Staphylococcus virus 80, Staphylococcus virus 85, Staphylococcus virus 88, Staphylococcus virus 92, Staphylococcus virus 96, Staphylococcus virus 187, Staphylococcus virus 52a, Staphylococcus virus 80alpha, Staphylococcus virus CNPH82, Staphylococcus virus EW, Staphylococcus virus IPLA5, Staphylococcus virus IPLA7, Staphylococcus virus IPLA88, Staphylococcus virus PH15, Staphylococcus virus phiETA, Staphylococcus virus phiETA2, Staphylococcus virus phiETA3, Staphylococcus virus phiMR11, Staphylococcus virus phiMR25, Staphylococcus virus phiNM1, Staphylococcus virus phiNM2, Staphylococcus virus phiNM4, Staphylococcus virus SAP26, Staphylococcus virus X2, Enterococcus virus FL1, Enterococcus virus FL2, Enterococcus virus FL3, Lactobacillus virus ATCC8014, Lactobacillus virus phiJL1, Pediococcus virus cP1, Aeromonas virus pIS4A, Listeria virus LP302, Listeria virus PSA, Methanobacterium virus psiM1, Roseobacter virus RDJL1, Roseobacter virus RDJL2, Rhodococcus virus RER2, Enterococcus virus BC611, Enterococcus virus IMEEF1, Enterococcus virus SAP6, Enterococcus virus VD13, Streptococcus virus SPQS1, Mycobacterium virus Papyrus, Mycobacterium virus Send513, Burkholderia virus KL1, Pseudomonas virus 73, Pseudomonas virus Ab26, Pseudomonas virus Kakheti25, Escherichia virus Cajan, Escherichia virus Seurat, Staphylococcus virus SEP9, Staphylococcus virus Sextaec, Streptococcus virus 858, Streptococcus virus 2972, Streptococcus virus ALQ132, Streptococcus virus 01205, Streptococcus virus Sfi11, Streptococcus virus 7201, Streptococcus virus DT1, Streptococcus virus phiAbc2, Streptococcus virus Sfi19, Streptococcus virus Sfi21, Paenibacillus virus Diva, Paenibacillus virus Hb10c2, Paenibacillus virus Rani, Paenibacillus virus Shelly, Paenibacillus virus Sitara, Paenibacillus virus Willow, Lactococcus virus 712, Lactococcus virus ASCC191, Lactococcus virus ASCC273, Lactococcus virus ASCC281, Lactococcus virus ASCC465, Lactococcus virus ASCC532, Lactococcus virus Bibb29, Lactococcus virus blL170, Lactococcus virus CB13, Lactococcus virus CB14, Lactococcus virus CB19, Lactococcus virus CB20, Lactococcus virus jj50, Lactococcus virus P2, Lactococcus virus P008, Lactococcus virus sk1, Lactococcus virus S14, Bacillus virus Slash, Bacillus virus Stahl, Bacillus virus Staley, Bacillus virus Stills, Gordonia virus Bachita, Gordonia virus ClubL, Gordonia virus OneUp, Gordonia virus Smoothie, Gordonia virus Soups, Bacillus virus SPbeta, Vibrio virus MAR10, Vibrio virus SSP002, Escherichia virus AKFV33, Escherichia virus BF23, Escherichia virus DT57C, Escherichia virus EPS7, Escherichia virus FFH1, Escherichia virus H8, Escherichia virus slur09, Escherichia virus T5, Salmonella virus 118970sal2, Salmonella virus Shivani, Salmonella virus SPC35, Salmonella virus Stitch, Arthrobacter virus Tank, Tsukamurella virus TIN2, Tsukamurella virus TIN3, Tsukamurella virus TIN4, Rhodobacter virus RcSpartan, Rhodobacter virus RcTitan, Mycobacterium virus Anaya, Mycobacterium virus Angelica, Mycobacterium virus Crimd, Mycobacterium virus Fionnbarth, Mycobacterium virus Jaws, Mycobacterium virus Larva, Mycobacterium virus Macncheese, Mycobacterium virus Pixie, Mycobacterium virus TM4, Bacillus virus BMBtp2, Bacillus virus TP21, Geobacillus virus Tp84, Staphylococcus virus 47, Staphylococcus virus 3a, Staphylococcus virus 42e, Staphylococcus virus IPLA35, Staphylococcus virus phi12, Staphylococcus virus phiSLT, Mycobacterium virus 32HC, Rhodococcus virus RGL3, Paenibacillus virus Vegas, Gordonia virus Vendetta, Bacillus virus Wbeta, Mycobacterium virus Wildcat, Gordonia virus Twister6, Gordonia virus Wizard, Gordonia virus Hotorobo, Gordonia virus Monty, Gordonia virus Woes, Xanthomonas virus CP1, Xanthomonas virus OP1, Xanthomonas virus phil7, Xanthomonas virus Xop411, Xanthomonas virus Xp10, Streptomyces virus TP1604, Streptomyces virus YDN12, Alphaproteobacteria virus phiJI001, Pseudomonas virus LKO4, Pseudomonas virus M6, Pseudomonas virus MP1412, Pseudomonas virus PAE1, Pseudomonas virus Yua, Pseudoalteromonas virus PM2, Pseudomonas virus phi6, Pseudomonas virus phi8, Pseudomonas virus phi12, Pseudomonas virus phi13, Pseudomonas virus phi2954, Pseudomonas virus phiNN, Pseudomonas virus phiYY, Vibrio virus fs1, Vibrio virus VGJ, Ralstonia virus RS603, Ralstonia virus RSM1, Ralstonia virus RSM3, Escherichia virus M13, Escherichia virus 122, Salmonella virus IKe, Acholeplasma virus L51, Vibrio virus fs2, Vibrio virus VFJ, Escherichia virus If1, Propionibacterium virus B5, Pseudomonas virus Pf1, Pseudomonas virus Pf3, Ralstonia virus PE226, Ralstonia virus RSS1, Spiroplasma virus SVTS2, Stenotrophomonas virus PSH1, Stenotrophomonas virus SMA6, Stenotrophomonas virus SMA7, Stenotrophomonas virus SMA9, Vibrio virus CTXphi, Vibrio virus KSF1, Vibrio virus VCY, Vibrio virus Vf33, Vibrio virus VfO3K6, Xanthomonas virus Cf1c, Spiroplasma virus C74, Spiroplasma virus R8A2B, Spiroplasma virus SkV1CR23x, Escherichia virus F1, Escherichia virus Qbeta, Escherichia virus BZ13, Escherichia virus MS2, Escherichia virus alpha3, Escherichia virus ID21, Escherichia virus ID32, Escherichia virus ID62, Escherichia virus NC28, Escherichia virus NC29, Escherichia virus NC35, Escherichia virus phiK, Escherichia virus St1, Escherichia virus WA45, Escherichia virus G4, Escherichia virus ID52, Escherichia virus Talmos, Escherichia virus phiX174, Bdellovibrio virus MAC1, Bdellovibrio virus MH2K, Chlamydia virus Chp1, Chlamydia virus Chp2, Chlamydia virus CPAR39, Chlamydia virus CPG1, Spiroplasma virus SpV4, Acholeplasma virus L2, Pseudomonas virus PR4, Pseudomonas virus PRD1, Bacillus virus AP50, Bacillus virus Bam35, Bacillus virus GIL16, Bacillus virus Wip1, Escherichia virus phi80, Escherichia virus RB42, Escherichia virus T2, Escherichia virus T3, Escherichia virus T6, Escherichia virus VT2-Sa, Escherichia virus VT1-Sakai, Escherichia virus VT2-Sakai, Escherichia virus CP-933V, Escherichia virus P27, Escherichia virus Stx2phi-I, Escherichia virus Stx1phi, Escherichia virus Stx2phi-II, Escherichia virus CP-1639, based on the Escherichia virus BP-4795, Escherichia virus 86, Escherichia virus Min27, Escherichia virus 2851, Escherichia virus 1717, Escherichia virus YYZ-2008, Escherichia virus EC026_P06, Escherichia virus ECO103_P15, Escherichia virus ECO103_P12, Escherichia virus ECO111_P16, Escherichia virus ECO111_P11, Escherichia virus VT2phi_272, Escherichia virus TL-2011c, Escherichia virus P13374, Escherichia virus Sp5.

In one embodiment, the bacterial virus particles typically target E. coli and include the capsid of a bacteriophage selected in the group consisting of BW73, B278, D6, D108, E, EI, E24, E41, FI-2, FI-4, FI-5, HI8A, Ffl8B, i, MM, Mu, 025, Phl-5, Pk, PSP3, PI, PID, P2, P4, SI, Wφ, φK13, φI, φ2, φ7, φ92, 7 A, 8φ, 9φ, 18, 28-1, 186, 299, HH-Escherichia (2), AB48, CM, C4, C16, DD-VI, E4, E7, E28, FII, F13, H, HI, H3, H8, K3, M, N, ND-2, ND-3, ND4, ND-5, ND6, ND-7, Ox-I, Ox-2, Ox-3, Ox-4, Ox-5, Ox-6, Phl-I, RB42, RB43, RB49, RB69, S, Sal-I, Sal-2, Sal-3, Sal-4, Sal-5, Sal-6, TC23, TC45, TulI*-6, TulP-24, TulI*46, TulP-60, T2, T4, T6, T35, al, 1, IA, 3, 3A, 3T+, 5φ, 9266Q, CFO103, HK620, J, K, KIF, m59, no. A, no. E, no. 3, no. 9, N4, sd, T3, T7, WPK, W31, ΔH, φC3888, φK3, φK7, φK12, φV-1, Φ04-CF, Φ05, Φ06, Φ07, φI, φI.2, φ20, φ95, φ263, φIO92, φI, φII, Ω8, 1, 3, 7, 8, 26, 27, 28-2, 29, 30, 31, 32, 38, 39, 42, 933W, NN-Escherichia (1), Esc-7-11, AC30, CVX-5, CI, DDUP, ECI, EC2, E21, E29, F1, F26S, F27S, Hi, HK022, HK97, HK139, HK253, HK256, K7, ND-I, PA-2, q, S2, TI, ), T3C, T5, UC-I, w, β4, γ2, λ, ΦD326, φγ, Φ06, Φ7, Φ10, φ80, χ, 2, 4, 4A, 6, 8A, 102, 150, 168, 174, 3000, AC6, AC7, AC28, AC43, AC50, AC57, AC81, AC95, HK243, KIO, ZG/3A, 5, 5A, 21EL, H19-J and 933H.

The present invention thus also concerns a bacterial delivery vehicle, as defined above, for use in in vivo delivery of a nucleic acid of interest into a targeted receiver bacterial cell, as defined above, wherein said bacterial delivery vehicle comprises the vector of the invention.

### Donor Bacterial Cell

In the context of the application, the term “donor bacterial cell” refers to a bacterial cell hosting a vector or a plasmid, to a production cell line or to a bacterium that is capable of transferring a conjugative plasmid to another bacterium.

The present invention also concerns a donor bacterial cell comprising the vector of the invention or producing the bacterial delivery vehicle of the invention, wherein said donor bacterial cell stably comprises the vector of the invention and is able to replicate said vector.

In a particular embodiment, when the conditional origin of replication of said vector is an origin of replication, the replication of which depends upon the presence of a given protein, peptid, nucleic acid, RNA, molecule or any combination thereof, said donor bacterial cell expresses said protein, peptid, nucleic acid, RNA, molecule or any combination thereof. Preferably, said protein, peptid, nucleic acid, RNA, molecule or any combination thereof is expressed in trans, as defined in the section “Conditional origin of replication” above.

In a particular embodiment, said donor bacterial cell stably comprises a nucleic acid encoding said protein, peptid, nucleic acid, RNA, molecule or any combination thereof.

In a particular embodiment, when said origin of replication is derived from phage-inducible chromosomal islands (PICIs), said conditional origin of replication is active in said donor bacterial cell because said donor bacterial cell expresses a rep protein, in particular a primase-helicase, in particular a primase-helicase of sequence SEQ ID NO: 8.

In a particular embodiment, said donor bacterial cell stably comprises a nucleic acid encoding said rep protein, in particular said primase-helicase, said nucleic acid typically comprising or consisting of the sequence SEQ ID NO: 9.

In a particular embodiment, said donor bacterial cell is a production cell line, in particular a cell line producing packaged phagemids including the vector of the invention.

Generation of packaged phagemids and bacteriophage particles by production cell lines are routine techniques well-known to one skilled in the art. In an embodiment, a satellite phage and/or helper phage may be used to promote the packaging of the vector in the delivery vehicles disclosed herein. Helper phages provide functions in trans and are well known to the man skilled in the art. The helper phage comprises all the genes coding for the structural and functional proteins that are indispensable for the phagemid to be packaged, (i.e. the helper phage provides all the necessary gene products for the assembly of the delivery vehicle). The helper phage may contain a defective origin of replication or packaging signal, or completely lack the latter, and hence it is incapable of self-packaging, thus only bacterial delivery particles carrying the vector or plasmid will be produced. Helper phages may be chosen so that they cannot induce lysis of the bacterial cells used for the delivery particle production. One skilled in the art would understand that some bacteriophages are defective and need a helper phage for payload packaging. Thus, depending on the bacteriophage chosen to prepare the bacterial delivery particles, the person skilled in the art would know if a helper phage is required. Sequences coding for one or more proteins or regulatory processes necessary for the assembly or production of packaged payloads may be supplied in trans. For example, STF, gpJ and gpH proteins may be provided in a plasmid under the control of an inducible promoter or expressed constitutively. In this case, the phage wild-type sequence may or not contain a deletion of the gene or sequence supplied in trans. Additionally, chimeric or modified phage sequences encoding a new function, like an engineered STF, gpJ or gpH protein, may be directly inserted into the desired position in the genome of the helper phage, hence bypassing the necessity of providing the modified sequence in trans. Methods for both supplying a sequence or protein in trans in the form of a plasmid, as well as methods to generate direct genomic insertions, modifications and mutations are well known to those skilled in the art.

In a particular embodiment, said helper phage comprises a nucleic acid sequence encoding a chimeric STF comprising or consisting of the sequence SEQ ID NO: 12, said nucleic acid sequence typically comprising or consisting of the sequence SEQ ID NO: 13, and said helper phage optionally further comprises a nucleic acid sequence encoding a chimeric gpJ variant comprising or consisting of the sequence SEQ ID NO: 14, said nucleic acid sequence typically comprising or consisting of the sequence SEQ ID NO: 15.

In a particular embodiment, said helper phage is a lambda prophage wherein (i) the nucleic acid encoding a wild-type STF protein has been replaced by a nucleic acid sequence encoding a chimeric STF comprising or consisting of the sequence SEQ ID NO: 12, said nucleic acid sequence typically comprising or consisting of the sequence SEQ ID NO: 13, (ii) the nucleic acid encoding a wild-type gpJ protein has been replaced by a nucleic acid sequence encoding a chimeric gpJ variant comprising or consisting of the sequence SEQ ID NO: 14, said nucleic acid sequence typically comprising or consisting of the sequence SEQ ID NO: 15, and (iii) the Cos site has been removed, and wherein optionally (iv) the helper prophage contains a mutation which prevents spontaneous cell lysis, such as the Sam7 mutation and (v) the helper prophage contains a thermosensitive version of the master cl repressor, such as the c1857 version.

In a particular embodiment, the donor bacterial cell of the invention comprises the above-defined helper phage.

### Treatment of Disease—Cosmetic Treatment

The vector used in the method of modulation of the invention may be administered as such, in a bacterial delivery vehicle or through a donor bacterial cell delivering said vector to the receiver bacterial cell. Said vector, bacterial delivery vehicle or donor bacterial cell may be more particularly administered in the form of a pharmaceutical or cosmetic composition comprising said vector, bacterial delivery vehicle or donor bacterial cell and a pharmaceutically acceptable carrier.

Generally, for pharmaceutical or cosmetic use, the vector, bacterial delivery vehicle or donor bacterial cell may be formulated as a pharmaceutical or cosmetic preparation or compositions comprising at least one vector, bacterial delivery vehicle or donor bacterial cell, and at least one pharmaceutically or cosmetically acceptable carrier, diluent or excipient, and optionally one or more further pharmaceutically or cosmetically active compounds. Such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. In a particular embodiment, said composition is for oral administration. Such administration forms may be solid, semi-solid or liquid, depending on the manner and route of administration. For example, formulations for oral administration may be provided with an enteric coating that will allow the vector, bacterial delivery vehicle or donor bacterial cell, in the formulation to resist the gastric environment and pass into the intestines. More generally, vector formulations, bacterial delivery vehicle formulations or donor bacterial cell formulations for oral administration may be suitably formulated for delivery into any desired part of the gastrointestinal tract. In addition, suitable suppositories may be used for delivery into the gastrointestinal tract. Various pharmaceutically or cosmetically acceptable carriers, diluents and excipients useful in pharmaceutical or veterinary or cosmetic compositions are known to the skilled person

The pharmaceutical or veterinary composition according to the invention may further comprise a pharmaceutically acceptable vehicle. The cosmetic composition of the invention may further comprise a cosmetically acceptable vehicle. A solid pharmaceutically or cosmetically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. Suitable solid vehicles include, for example calcium phosphate, magnesium stearate, talc, sugars, lactose, dextrin, starch, gelatin, cellulose, polyvinylpyrrolidone, low melting waxes and ion exchange resins.

The pharmaceutical or veterinary or cosmetic composition may be prepared as a sterile solid composition that may be suspended at the time of administration using sterile water, saline, or other appropriate sterile injectable medium. The pharmaceutical or veterinary or cosmetic compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 8o (oleate esters of sorbitol and its anhydrides copolymerized with ethylene oxide) and the like. The particles according to the disclosure can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for enteral administration include sterile solutions, emulsions, and suspensions.

The vectors, bacterial delivery vehicles or donor bacterial cells disclosed herein may be dissolved or suspended in a pharmaceutically or cosmetically acceptable liquid vehicle such as water, an organic solvent, a mixture of both or pharmaceutically acceptable oils or fats. The liquid vehicle can contain other suitable pharmaceutical or cosmetic additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and enteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for enteral administration. The liquid vehicle for pressurized compositions can be a halogenated hydrocarbon or other pharmaceutically or cosmetically acceptable propellant.

In some embodiments, the invention encompasses pharmaceutical or veterinary or cosmetic composition formulated for delayed or gradual enteric release. In preferred embodiments, formulations or pharmaceutical or cosmetic preparations of the invention are formulated for delivery of the vector into the distal small bowel and/or the colon. The formulation can allow the vector to pass through stomach acid and pancreatic enzymes and bile, and reach undamaged to be viable in the distal small bowel and colon.

In some embodiments, the pharmaceutical or veterinary or cosmetic composition is micro-encapsulated, formed into tablets and/or placed into capsules, preferably enteric-coated capsules.

In some embodiments, the pharmaceutical or veterinary or cosmetic compositions are formulated for delayed or gradual enteric release, using cellulose acetate (CA) and polyethylene glycol (PEG). In some embodiments, the pharmaceutical or veterinary or cosmetic compositions are formulated for delayed or gradual enteric release using a hydroxypropylmethylcellulose (HPMC), a microcrystalline cellulose (MCC) and magnesium stearate. the pharmaceutical or veterinary compositions are formulated for delayed or gradual enteric release using e.g., a poly(meth)acrylate, e.g. a methacrylic acid copolymer B, a methyl methacrylate and/or a methacrylic acid ester, or a polyvinylpyrrolidone (PVP).

In some embodiments, the pharmaceutical or veterinary or cosmetic compositions are formulated for delayed or gradual enteric release using a release-retarding matrix material such as: an acrylic polymer, a cellulose, a wax, a fatty acid, shellac, zein, hydrogenated vegetable oil, hydrogenated castor oil, polyvinylpyrrolidone, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, polyvinylalcohols, polyvinylalcohol copolymers, polyethylene glycols, non-crosslinked polyvinylpyrrolidone, polyvinyl acetates, polyvinylacetate copolymers or any combination thereof.

In some embodiments, the pharmaceutical or veterinary compositions are formulated for delayed or gradual enteric release as described in U.S. Pat. App. Pub. 20110218216, which describes an extended release pharmaceutical composition for oral administration, and uses a hydrophilic polymer, a hydrophobic material and a hydrophobic polymer or a mixture thereof, with a microenvironment pH modifier. The hydrophobic polymer can be ethylcellulose, cellulose acetate, cellulose propionate, cellulose butyrate, methacrylic acid-acrylic acid copolymers or a mixture thereof. The hydrophilic polymer can be polyvinylpyrrolidone, hydroxypropylcellulose, methylcellulose, hydroxypropylmethyl cellulose, polyethylene oxide, acrylic acid copolymers or a mixture thereof. The hydrophobic material can be a hydrogenated vegetable oil, hydrogenated castor oil, carnauba wax, candellia wax, beeswax, paraffin wax, stearic acid, glyceryl behenate, cetyl alcohol, cetostearyl alcohol or and a mixture thereof. The microenvironment pH modifier can be an inorganic acid, an amino acid, an organic acid or a mixture thereof. Alternatively, the microenvironment pH modifier can be lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid, a salicylic acid, tosylic acid, mesylic acid or malic acid or a mixture thereof.

In some embodiments, the pharmaceutical or veterinary or cosmetic compositions are a powder that can be included into a tablet or a suppository. In alternative embodiments, a formulation or pharmaceutical or cosmetic preparation of the invention can be a ‘powder for reconstitution’ as a liquid to be drunk or otherwise administered.

In some embodiments, the pharmaceutical or veterinary or cosmetic compositions can be administered in a cream, gel, lotion, liquid, feed, or aerosol spray. In some embodiments, a bacteriophage is immobilized to a solid surface using any substance known in the art and any technology known in the art, for example, but not limited to immobilization of bacteriophages onto polymeric beads using technology as outlined in U.S. Pat. No. 7,482,115, which is incorporated herein by reference. Phages may be immobilized onto appropriately sized polymeric beads so that the coated beads may be added to aerosols, creams, gels or liquids. The size of the polymeric beads may be from about 0.1 μm to 500 μm, for example 50 μm to 100 μm. The coated polymeric beads may be incorporated into animal feed, including pelleted feed and feed in any other format, incorporated into any other edible devise used to present phage to the animals, added to water offered to animals in a bowl, presented to animals through water feeding systems. In some embodiments, the compositions are used for treatment of surface wounds and other surface infections using creams, gels, aerosol sprays and the like.

In some embodiments, the pharmaceutical or veterinary or cosmetic compositions can be administered by inhalation, in the form of a suppository or pessary, topically (e.g., in the form of a lotion, solution, cream, ointment or dusting powder), epi- or transdermally (e.g., by use of a skin patch), orally (e.g., as a tablet, which may contain excipients such as starch or lactose), as a capsule, ovule, elixirs, solutions, or suspensions (each optionally containing flavoring, coloring agents and/or excipients), or they can be injected parenterally (e.g., intravenously, intramuscularly or subcutaneously). For parenteral administration, the compositions may be used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner. In a preferred embodiment, a bacteriophage and/or polypeptide of the present invention is administered topically, either as a single agent, or in combination with other antibiotic treatments, as described herein or known in the art.

In some embodiments, the pharmaceutical or veterinary or cosmetic compositions can also be dermally or transdermally administered. For topical application to the skin, the pharmaceutical or veterinary or cosmetic composition can be combined with one or a combination of carriers, which can include but are not limited to, an aqueous liquid, an alcohol base liquid, a water soluble gel, a lotion, an ointment, a nonaqueous liquid base, a mineral oil base, a blend of mineral oil and petrolatum, lanolin, liposomes, proteins carriers such as serum albumin or gelatin, powdered cellulose carmel, and combinations thereof. A topical mode of delivery may include a smear, a spray, a bandage, a time-release patch, a liquid-absorbed wipe, and combinations thereof. The pharmaceutical or veterinary or cosmetic composition can be applied to a patch, wipe, bandage, etc., either directly or in a carrier(s). The patches, wipes, bandages, etc., may be damp or dry, wherein the phage and/or polypeptide (e.g., a lysin) is in a lyophilized form on the patch. The carriers of topical compositions may comprise semi-solid and gel-like vehicles that include a polymer thickener, water, preservatives, active surfactants, or emulsifiers, antioxidants, sun screens, and a solvent or mixed solvent system. U.S. Pat. No. 5,863,560 discloses a number of different carrier combinations that can aid in the exposure of skin to a medicament, and its contents are incorporated herein.

For intranasal or administration by inhalation, the pharmaceutical or veterinary or cosmetic composition is conveniently delivered in the form of a dry powder inhaler or an aerosol spray presentation from a pressurized container, pump, spray, or nebuliser with the use of a suitable propellant, e.g., dichlorodifluoromethane, trichlorofluoromethane, dichlorotetrafluoroethane, a hydrofluoroalkane such as 1,1,1,2-tetrafluoroethane or 1,1,1,2,3,3,3-heptafluoropropane, carbon dioxide, or other suitable gas. In the case of a pressurized aerosol, the dosage unit may be determined by providing a valve to deliver a metered amount. The pressurized container, pump, spray, or nebuliser may contain a solution or suspension of the active compound, e.g., using a mixture of ethanol and the propellant as the solvent, which may additionally contain a lubricant, e.g., sorbitan trioleate. Capsules and cartridges (made, for example, from gelatin) for use in an inhaler or insufflator may be formulated to contain a powder mix of the bacteriophage and/or polypeptide of the invention and a suitable powder base such as lactose or starch.

For administration in the form of a suppository or pessary, the pharmaceutical or veterinary composition can be applied topically in the form of a gel, hydrogel, lotion, solution, cream, ointment, or dusting powder. Compositions of the invention may also be administered by the ocular route. For ophthalmic use, the compositions of the invention can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

Dosages and desired drug concentrations of the pharmaceutical and veterinary composition compositions of the present invention may vary depending on the particular use. The determination of the appropriate dosage or route of administration is within the skill of an ordinary physician. Animal experiments can provide reliable guidance for the determination of effective doses in human therapy.

For transdermal administration, the pharmaceutical or veterinary composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compounds can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

In a particular embodiment, the composition of the invention may further comprise at least one additional active ingredient, for instance a prebiotic and/or a probiotic and/or an antibiotic, and/or another antibacterial or antibiofilm agent, and/or any agent enhancing the targeting of the vector to a bacteria and/or the delivery of the vector into a bacteria.

As used herein, a “prebiotic” refers to an ingredient that allows specific changes, both in the composition and/or activity in the gastrointestinal microbiota that may confer benefits upon the host. A prebiotic can be a comestible food or beverage or ingredient thereof. A prebiotic may be a selectively fermented ingredient. Prebiotics may include complex carbohydrates, amino acids, peptides, minerals, or other essential nutritional components for the survival of the bacterial composition. Prebiotics include, but are not limited to, amino acids, biotin, fructo-oligosaccharide, galacto-oligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-1), dietary fibers (e.g., soy fiber, sugarbeet fiber, pea fiber, corn bran, and oat fiber) and xylooligosaccharides.

As used herein, a “probiotic” refers to a dietary supplement based on living microbes which, when taken in adequate quantities, has a beneficial effect on the host organism by strengthening the intestinal ecosystem. Probiotic can comprise a non-pathogenic bacterial or fungal population, e.g., an immunomodulatory bacterial population, such as an anti-inflammatory bacterial population, with or without one or more prebiotics. They contain a sufficiently high number of living and active probiotic microorganisms that can exert a balancing action on gut flora by direct colonisation. It must be noted that, for the purposes of the present description, the term “probiotic” is taken to mean any biologically active form of probiotic, preferably including but not limited to lactobacilli, bifidobacteria, streptococci, enterococci, propionibacteria or saccharomycetes but even other microorganisms making up the normal gut flora, or also fragments of the bacterial wall or of the DNA of these microorganisms. These compositions are advantageous in being suitable for safe administration to humans and other mammalian subjects and are efficacious for the treatment, prevention, of a disease or disorder caused by bacteria such as bacterial infection. Probiotics include, but are not limited to lactobacilli, bifidobacteria, streptococci, enterococci, propionibacteria, saccharomycetes, lactobacilli, bifidobacteria, or proteobacteria.

In a particular embodiment, said probiotic is not affected by the vector of the invention. In a particular embodiment, when said vector is comprised in a bacterial delivery vehicle, said vehicle may bind to said probiotic but said probiotic is not affected by said vector. In an alternative embodiment, when said vector is comprised in a bacterial delivery vehicle, said vehicle does not bind to said probiotic and said probiotic is not affected by said vector.

In a particular embodiment, the effect of said vector induces or increases a synergy with the effect of the additional active ingredient. In a more particular embodiment, said vector enables said probiotic to engraft into said host organism.

The antibiotic can be selected from the group consisting of penicillins such as penicillin G, penicillin K, penicillin N, penicillin O, penicillin V, methicillin, benzylpenicillin, nafcillin, oxacillin, cloxacillin, dicloxacillin, ampicillin, amoxicillin, pivampicillin, hetacillin, bacampicillin, metampicillin, talampicillin, epicillin, carbenicillin, ticarcillin, temocillin, mezlocillin, and piperacillin; cephalosporins such as cefacetrile, cefadroxil, cephalexin, cefaloglycin, cefalonium, cephaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, loracarbef, cefbuperazone, cefminox, cefotetan, cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, latamoxef, cefclidine, cefepime, cefluprenam, cefoselis, cefozopran, cefpirome, cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; polymyxins such as polysporin, neosporin, polymyxin B, and polymyxin E, rifampicins such as rifampicin, rifapentine, and rifaximin; Fidaxomicin; quinolones such as cinoxacin, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, temafloxacin, tosufloxacin, clinafloxacin, gatifloxacin, gemifloxacin, moxifloxacin, sitafloxacin, trovafloxacin, prulifloxacin, delafloxacin, nemonoxacin, and zabofloxacin; sulfonamides such as sulfafurazole, sulfacetamide, sulfadiazine, sulfadimidine, sulfafurazole, sulfisomidine, sulfadoxine, sulfamethoxazole, sulfamoxole, sulfanitran, sulfadimethoxine, sulfametho-xypyridazine, sulfametoxydiazine, sulfadoxine, sulfametopyrazine, and terephtyl; macrolides such as azithromycin, clarithromycin, erythromycin, fidaxomicin, telithromycin, carbomycin A, josamycin, kitasamycin, midecamycin, oleandomycin, solithromycin, spiramycin, troleandomycin, tylosin, and roxithromycin; ketolides such as telithromycin, and cethromycin; fluoroketolides such as solithromycin; lincosamides such as lincomycin, clindamycin, and pirlimycin; tetracyclines such as demeclocycline, doxycycline, minocycline, oxytetracycline, and tetracycline; aminoglycosides such as amikacin, dibekacin, gentamicin, kanamycin, neomycin, netilmicin, sisomicin, tobramycin, paromomycin, and streptomycin; ansamycins such as geldanamycin, herbimycin, and rifaximin; carbacephems such as loracarbef; carbapenems such as ertapenem, doripenem, imipenem (or cilastatin), and meropenem; glycopeptides such as teicoplanin, vancomycin, telavancin, dalbavancin, and oritavancin; lincosamides such as clindamycin and lincomycin; lipopeptides such as daptomycin; monobactams such as aztreonam; nitrofurans such as furazolidone, and nitrofurantoin; oxazolidinones such as linezolid, posizolid, radezolid, and torezolid; teixobactin, clofazimine, dapsone, capreomycin, cycloserine, ethambutol, ethionamide, isoniazid, pyrazinamide, rifabutin, arsphenamine, chloramphenicol, fosfomycin, fusidic acid, metronidazole, mupirocin, platensimycin, quinupristin (or dalfopristin), thiamphenicol, tigecycline, tinidazole, trimethoprim, alatrofloxacin, fidaxomicin, nalidixic acid, rifampin, derivatives and combination thereof.

In a particular embodiment, the modulating method of the invention is for treating and/or preventing a disease in said host subject.

In a particular embodiment, said disease is caused or mediated by bacteria.

The diseases or disorders caused or mediated by bacteria may be selected from the group consisting of skin chronic inflammation such as acne (acne vulgaris), progressive macular hypomelanosis, abdominal cramps, acute epiglottitis, arthritis, bacteraemia, bloody diarrhea, botulism, Brucellosis, brain abscess, cardiomyopathy, chancroid venereal disease, Chlamydia, Crohn's disease, conjunctivitis, cholecystitis, colorectal cancer, polyposis, dysbiosis, Lyme disease, diarrhea, diphtheria, duodenal ulcers, endocarditis, erysipelothricosis, enteric fever, fever, glomerulonephritis, gastroenteritis, gastric ulcers, Guillain-Barre syndrome tetanus, gonorrhoea, gingivitis, inflammatory bowel diseases, irritable bowel syndrome, leptospirosis, leprosy, listeriosis, tuberculosis, Lady Windermere syndrome, Legionaire's disease, meningitis, mucopurulent conjunctivitis, multi-drug resistant bacterial infections, multi-drug resistant bacterial carriage, myocarditis, myonecrosis-gas gangrene, Mycobacterium avium complex, neonatal necrotizing enterocolitis, nocardiosis, nosocomial infection, otitis, periodontitis, phalyngitis, pneumonia, peritonitis, purpuric fever, Rocky Mountain spotted fever, shigellosis, syphilis, sinusitis, sigmoiditis, septicaemia, subcutaneous abscesses, tularaemia, tracheobronchitis, tonsillitis, typhoid fever, ulcerative colitis, urinary infection, whooping cough, Nonalcoholic Fatty Liver Disease (NAFLD), Nonalcoholic steatohepatitis (NASH).

The infection caused by bacteria may be selected from the group consisting of infections, preferably intestinal infections such as esophagitis, gastritis, enteritis, colitis, sigmoiditis, rectitis, and peritonitis, urinary tract infections, vaginal infections, female upper genital tract infections such as salpingitis, endometritis, oophoritis, myometritis, parametritis and infection in the pelvic peritoneum, respiratory tract infections such as pneumonia, intra-amniotic infections, odontogenic infections, endodontic infections, fibrosis, meningitis, bloodstream infections, nosocomial infection such as catheter-related infections, hospital acquired pneumonia, postpartum infection, hospital acquired gastroenteritis, hospital acquired urinary tract infections, or a combination thereof. Preferably, the infection according to the invention is caused by a bacterium presenting an antibiotic resistance. In a particular embodiment, the infection is caused by a bacterium as listed above in the targeted bacteria.

The disclosure also concerns a pharmaceutical or veterinary composition of the invention for the treatment of a metabolic disorder including, for example, obesity, type 2 diabetes and nonalcoholic fatty liver disease. Indeed, emerging evidence indicates that these disorders are characterized by alterations in the intestinal microbiota composition and its metabolites (Tilg et al., Nature Reviews Immunology, volume 20, pages 40-54, 2020). The pharmaceutical or veterinary composition may thus be used to deliver in some intestinal bacteria a nucleic acid of interest which can alter the intestinal microbiota composition or its metabolites (e.g. by inducing expression, overexpression or secretion of some molecules by said bacteria, for example molecules having a beneficial role on metabolic inflammation). The disclosure also concerns the use of a pharmaceutical or veterinary composition of the invention for the manufacture of a medicament for the treatment of a metabolic disorder including, for example, obesity, type 2 diabetes and nonalcoholic fatty liver disease. It also relates to a method for treating a metabolic disorder including, for example, obesity, type 2 diabetes and nonalcoholic fatty liver disease, comprising administering to a subject having a metabolic disorder in need of treatment the provided pharmaceutical or veterinary composition, in particular a therapeutically effective amount of the provided pharmaceutical or veterinary composition.

In a particular embodiment, the invention concerns a pharmaceutical or veterinary composition for use in the treatment of pathologies involving bacteria of the human microbiome, such as inflammatory and auto-immune diseases, cancers, infections or brain disorders. Indeed, some bacteria of the microbiome, without triggering any infection, can secrete molecules that will induce and/or enhance inflammatory or auto-immune diseases or cancer development. More specifically, the present invention relates also to modulating microbiome composition to improve the efficacy of immunotherapies based, for example, on CAR-T (Chimeric Antigen Receptor T) cells, TIL (Tumor Infiltrating Lymphocytes) and Tregs (Regulatory T cells) also known as suppressor T cells. Modulation of the microbiome composition to improve the efficacy of immunotherapies may also include the use of immune checkpoint inhibitors well known in the art such as, without limitation, PD-1 (programmed cell death protein 1) inhibitor, PD-L1 (programmed death ligand 1) inhibitor and CTLA-4 (cytotoxic T lymphocyte associated protein 4).

In an alternative embodiment, said disease is not caused by bacteria.

In certain embodiments, the disease to be treated is cancer or a proliferative disorder, including but not limited to, breast cancer (e.g., triple negative breast cancer, ER+ breast cancer, or ER− breast cancer), basal cell carcinoma, skin cancer, lung cancer, small cell lung cancer, non-small cell lung cancer, brain cancer, medulloblastoma, glioma (including glioblastoma, oligodendroglioma, astrocytoma, ependymoma), neuroblastoma, colorectal cancer, ovarian cancer, liver cancer, pancreatic cancer (e.g., carcinoma, angiosarcoma, adenosarcoma), gastric cancer, gastroesophageal junction cancer, prostate cancer, cervical cancer, bladder cancer, head and neck cancer, lymphoma (e.g., mantle cell lymphoma, diffuse large B-cell lymphoma), solid tumors that cannot be removed by surgery, locally advanced solid tumors, metastatic solid tumors, leukemia (e.g., acute myeloid leukemia (AML), acute lymphoblastic leukemia (ALL), or chronic myeloid leukemia (CML)), or recurrent or refractory tumors.

In one embodiment, the diseases to be treated include, but are not limited to, inflammatory or allergic diseases, including systemic anaphylaxis and hypersensitivity disorders, atopic dermatitis, urticaria, drug allergies, insect sting allergies, food allergies (including celiac disease and the like), and mastocytosis; inflammatory bowel diseases, including Crohn's disease, ulcerative colitis, ileitis, and enteritis; vasculitis, and Behcet's syndrome; psoriasis and inflammatory dermatoses, including dermatitis, eczema, atopic dermatitis, allergic contact dermatitis, urticaria, viral cutaneous pathologies including those derived from human papillomavirus, HIV or RLV infection, bacterial, flugal, and other parasital cutaneous pathologies, and cutaneous lupus erythematosus; asthma and respiratory allergic diseases, including allergic asthma, exercise induced asthma, allergic rhinitis, otitis media, allergic conjunctivitis, hypersensitivity lung diseases, and chronic obstructive pulmonary disease; autoimmune diseases, including arthritis (including rheumatoid and psoriatic), systemic lupus erythematosus, type I diabetes, myasthenia gravis, multiple sclerosis, Graves' disease, and glomerulonephritis; graft rejection (including allograft rejection and graft-v-host disease), e.g., skin graft rejection, solid organ transplant rejection, bone marrow transplant rejection; fever; cardiovascular disorders, including acute heart failure, hypotension, hypertension, angina pectoris, myocardial infarction, cardiomyopathy, congestive heart failure, atherosclerosis, coronary artery disease, restenosis, and vascular stenosis; cerebrovascular disorders, including traumatic brain injury, stroke, ischemic reperfusion injury and aneurysm; fibrosis, connective tissue disease, and sarcoidosis, genital and reproductive conditions, including erectile dysfunction; gastrointestinal disorders, including gastritis, ulcers, nausea, pancreatitis, and vomiting; neurologic disorders, including Alzheimer's disease; sleep disorders, including insomnia, narcolepsy, sleep apnea syndrome, and Pickwick Syndrome; pain; renal disorders; ocular disorders, including glaucoma; and non-bacterial infectious diseases, including HIV.

In some aspects, the disease to be treated may be an autoimmune disease such as autoimmune hemolytic anemia, autoimmune neonatal thrombocytopenia, autoimmune neutropenia, autoimmunocytopenia, antiphospholipid syndrome, dermatitis, gluten-sensitive enteropathy, allergic encephalomyelitis, myocarditis, relapsing polychondritis, rheumatic heart disease, glomerulonephritis, Multiple Sclerosis, Neuritis, Uveitis Ophthalmia, Polyendo-crinopathies, Purpura, Reiter's Disease, Stiff-Man Syndrome, Autoimmune Pulmonary Inflammation, myocarditis, IgA glomerulonephritis, dense deposit disease, rheumatic heart disease, Guillain-Barre Syndrome, insulin dependent diabetes mellitis, autoimmune inflammatory eye, autoimmune thyroiditis, hypothyroidism, systemic lupus erythematosus, discoid lupus, Goodpasture's syndrome, Pemphigus, Graves' Disease, Myasthenia Gravis, and insulin resistance, autoimmune hemolytic anemia, autoimmune thrombocytopenic purpura, rheumatoid arthritis, schleroderma with anti-collagen antibodies, mixed connective tissue disease, polymyositis/dermatomyositis, pernicious anemia, idiopathic Addison's disease, infertility, glomerulonephritis, bullous pemphigoid, Sjogren's syndrome, diabetes mellitus, adrenergic drug resistance with asthma or cystic fibrosis, chronic active hepatitis, primary biliary cirrhosis, endocrine gland failure, vitiligo, vasculitis, post-MI, cardiotomy syndrome, urticaria, atopic dermatitis, asthma, inflammatory myopathies, an inflammatory disorder, a granulomatous disorder, an atrophic disorder, or an alloimmune disease.

The subject to be treated may have been diagnosed with, or may be at risk of developing an infection, a disorder and/or a disease preferably due to a bacterium. Diagnostic method of such infection, disorder and/or disease are well known by the man skilled in the art.

In a particular embodiment, the infection, disorder and/or disease presents a resistance to treatment, preferably the infection, disorder or disease presents an antibiotic resistance.

In a particular embodiment, the subject has never received any treatment prior to the administration of the vectors according to the invention, particularly a vector packaged into a delivery vehicle according to the invention, preferably a packaged plasmid or phagemid into a bacterial virus particle according to the invention, or of a pharmaceutical or veterinary composition according to the invention.

In a particular embodiment, the subject has already received at least one line of treatment, preferably several lines of treatment, prior to the administration of the vectors according to the invention, particularly a vector packaged into a delivery vehicle according to the invention, preferably a packaged plasmid or phagemid into a bacterial virus particle according to the invention, or of a pharmaceutical or veterinary composition according to the invention.

Preferably, the treatment is administered regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

The duration of treatment with vectors according to the invention, particularly a vector packaged into a delivery vehicle according to the invention, preferably a packaged plasmid or phagemid into a bacterial virus particle according to the invention, or with a pharmaceutical or veterinary composition according to the invention, is preferably comprised between 1 day and 20 weeks, more preferably between 1 day and 10 weeks, still more preferably between 1 day and 4 weeks, even more preferably between 1 day and 2 weeks. In a particular embodiment, the duration of the treatment is of about 1 week. Alternatively, the treatment may last as long as the infection, disorder and/or disease persists.

The form of the pharmaceutical or veterinary compositions, the route of administration and the dose of administration of vectors according to the invention, particularly of a vector packaged into a delivery vehicle according to the invention, preferably of a packaged plasmid or phagemid into a bacterial virus particle according to the invention, or of a pharmaceutical or veterinary composition according to the invention can be adjusted by the man skilled in the art according to the type and severity of the infection (e.g. depending on the bacteria species involved in the disease, disorder and/or infection and its localization in the patient's or subject's body), and to the patient or subject, in particular its age, weight, sex, and general physical condition.

Particularly, the amount of vectors according to the invention, particularly a vector packaged into a delivery vehicle according to the invention, preferably a packaged plasmid or phagemid into a bacterial virus particle according to the invention, or of a pharmaceutical or veterinary composition according to the invention, to be administered has to be determined by standard procedure well known by those of ordinary skills in the art. Physiological data of the patient or subject (e.g. age, size, and weight) and the routes of administration have to be taken into account to determine the appropriate dosage, so as a therapeutically effective amount will be administered to the patient or subject.

For example, the total amount of vectors, particularly a vector packaged into a delivery vehicle according to the invention, preferably a plasmid or phagemid packaged into a bacterial virus particle according to the invention, for each administration is comprised between 10⁴ and 10¹⁵ delivery vehicles.

In another particular embodiment, the modulating method of the invention is for the cosmetic treatment of said host subject.

### Plant Treatment and Other Applications

In another particular embodiment, the host organism is a plant, and the modulating method of the invention is for the agronomical, prophylactic or phytotherapeutic treatment of said host plant.

In a particular embodiment, said modulating method is for improving the growth of said host plants, for preventing a disease or for treating diseases affecting said host plants.

The present invention also concerns a method for ex vivo modulating a microbiome from an environment by collecting targeted receiver bacterial cell from said environment and by delivering a nucleic acid of interest into said targeted receiver bacterial cell of said microbiome, said nucleic acid of interest producing a given effect, as disclosed above, on said targeted receiver bacterial cell,
wherein said method comprises contacting a nucleic acid vector comprising said nucleic acid of interest with said microbiome,
   wherein said vector further comprises a conditional origin of replication which is inactive in the targeted receiver bacterial cell but is active in a donor bacterial cell, and said vector is devoid of antibiotic resistance marker,
thereby delivering said nucleic acid of interest into the targeted receiver bacterial cell, and
wherein, once delivered into said targeted receiver bacterial cell, said nucleic acid of interest produces said given effect on said targeted receiver bacterial cell while said vector is not replicated in said targeted receiver bacterial cell.

By “environment” is meant herein all the elements which surround a species and among which some directly or indirectly contribute to the subsistence of said species. In a particular embodiment, said environment is not an animal. In an alternative embodiment, said environment is an animal, in particular a human.

In a particular embodiment, said environment can be any medium wherein said microbiome lives, such as a solid or semi-solid surface or a liquid medium, such as water, in particular waste water.

In a particular embodiment, said ex vivo method is for protecting a surface against biofouling. In another particular embodiment, said ex vivo method is for decontaminating water.

The present invention further concerns the following embodiments.
1. A method for in vivo modulating the microbiome of a host organism by delivering a nucleic acid of interest into a targeted receiver bacterial cell of said microbiome, said nucleic acid of interest producing a given effect on said targeted receiver bacterial cell,
   wherein said method comprises administering, in said organism, a nucleic acid vector comprising said nucleic acid of interest,
      wherein said vector further comprises a conditional origin of replication which is inactive in the targeted receiver bacterial cell but is active in a donor bacterial cell, and said vector is devoid of antibiotic resistance marker,
   thereby delivering said nucleic acid of interest into the targeted receiver bacterial cell, and
   wherein, once delivered into said targeted receiver bacterial cell, said nucleic acid of interest produces said given effect on said targeted receiver bacterial cell while said vector is not replicated in said targeted receiver bacterial cell.
2. The method according to embodiment 1, wherein said nucleic acid of interest is expressed in said targeted receiver bacterial, thereby producing said given effect.
3. The method according to embodiment 1 or 2, wherein said modulation of the microbiome is a modulation of the microbiome function or of the microbiome composition.
4. The method according to any one of embodiments 1 to 3, wherein said given effect is selected from the group consisting of killing the receiver bacterial cell, making the receiver bacterial cell stop producing a given molecule and making the receiver bacterial cell produce a molecule of interest.
5. The method according to any one of embodiments 1 to 4, wherein said given effect is making the receiver bacterial cell produce a molecule of interest and said molecule of interest is a host modulatory molecule.
6. The method according to embodiment 5, wherein said host modulatory molecule is selected from the group consisting of non-coding nucleic acids, coding nucleic acids, proteins, lipids, sugars, LPS, metabolites and small molecules.
7. The method according to any one of embodiments 5 to 6, wherein said host modulatory molecule is selected from the group consisting of host endogenous molecules, host exogenous molecules expressed naturally by other organisms, and synthetic compounds.
8. The method according to any one of embodiments 5 to 7, wherein said host modulatory molecule is selected from the group consisting of secreted molecules, intracellular molecules and membrane-displayed molecules.
9. The method according to any one of embodiments 5 to 8, wherein said molecule of interest is encoded by a nucleic acid selected from the group consisting of a gene encoding said host modulatory molecule, several genes encoding a protein complex that is the host modulatory molecule, a gene or group of genes encoding enzyme(s) of a metabolic pathway leading to the production of the host modulatory molecule, a coding nucleic acid which is the host modulatory molecule, and a non-coding nucleic acid which is the host modulatory molecule.
10. The method according to any one of embodiments 1 to 4, wherein said given effect is making the receiver bacterial cell stop producing a given molecule and wherein said given molecule is selected from the group consisting of a toxin, a toxic factor, a virulence protein, a virulence factor, a protein encoded by an antibiotic resistance gene, a protein encoded by a remodeling gene or by a modulatory gene.
11. The method according to embodiment 10, wherein said nucleic acid of interest is a gene or group of genes encoding one or more exogenous enzyme(s) which result(s) in a genetic modification.
12. The method according to embodiment 11, wherein said nucleic acid of interest is gene encoding a base-editor or a prime-editor.
13. The method according to any one of embodiments 1 to 4, wherein said given effect is killing the receiver bacterial cell and wherein said nucleic acid of interest is a gene encoding a nuclease.
14. The method according to any one of embodiments 1 to 13, wherein the conditional origin of replication is an origin of replication, the replication of which depends upon the presence of a given protein, peptid, nucleic acid, RNA, molecule or any combination thereof.
15. The method according to embodiment 14, wherein said conditional origin of replication is active in said donor bacterial cell because said donor bacterial cell expresses said given protein, peptid, nucleic acid, RNA, molecule or any combination thereof.
16. The method according to embodiment 15, wherein said protein, peptid, RNA, molecule or any combination thereof is expressed in trans in said donor bacterial cell.
17. The method according to embodiment 14 or 15, wherein said conditional origin of replication is an origin of replication derived from phage-inducible chromosomal islands (PICIs).
18. The method according to embodiment 17, wherein said conditional origin of replication is active in said donor bacterial cell because said donor bacterial cell expresses a rep protein, in particular a primase-helicase.
19. The method according to embodiment 17 or 18, wherein said conditional origin of replication is derived from the origin of replication from the PICI of the Escherichia coli strain CFT073.
20. The method according to any one of embodiments 1 to 19, wherein said vector does not comprise any restriction site of restriction enzymes which are frequently encoded in said targeted receiver bacterial cell.
21. The method according to any one of embodiments 1 to 20, for treating a disease in said host subject.
22. The method according to any one of embodiments 1 to 20, for a cosmetic treatment of said host subject.
23. A nucleic acid vector for use in in vivo delivery of a nucleic acid of interest into a targeted receiver bacterial cell, said nucleic acid of interest producing a given effect on said targeted receiver bacterial cell,
   wherein said vector comprises:
      said nucleic acid of interest, and
      a conditional origin of replication which is inactive in the targeted receiver bacterial cell but is active in a donor bacterial cell, and
   wherein said vector is devoid of antibiotic resistance marker.
24. The nucleic acid vector according to embodiment 23, wherein said conditional origin of replication is the primase on from the PICI of the Escherichia coli strain CFT073 or a derivative thereof.
25. The nucleic acid vector according to embodiment 23, wherein said conditional origin of replication comprises or consists of the sequence SEQ ID NO: 6 or SEQ ID NO: 7.
26. A bacterial delivery vehicle for use in in vivo delivery of a nucleic acid of interest into a targeted receiver bacterial cell, wherein said bacterial delivery vehicle comprises the vector according to any one of embodiments 23 to 25.
27. A donor cell line comprising the vector according to any one of embodiments 23 to 25 or producing the bacterial delivery vehicle according to embodiment 26, wherein said donor cell line stably comprises the vector according to any one of embodiments 23 to 25 and is able to replicate said vector.
28. The donor cell line according to embodiment 27, wherein the conditional origin of replication of said vector is an origin of replication, the replication of which depends upon the presence of a given protein, peptid, nucleic acid, RNA, molecule or any combination thereof, and said donor cell line expresses said protein, peptid, nucleic acid, RNA, molecule or any combination thereof.
29. The donor cell line according to embodiment 26, wherein said protein, peptid, nucleic acid, RNA, molecule or any combination thereof is expressed in trans.
30. A method for ex vivo modulating a microbiome from an environment by collecting targeted receiver bacterial cell from said environment and by delivering a nucleic acid of interest into a targeted receiver bacterial cell of said microbiome, said nucleic acid of interest producing a given effect on said targeted receiver bacterial cell,
   wherein said method comprises contacting a nucleic acid vector comprising said nucleic acid of interest with said microbiome,
   wherein said vector further comprises a conditional origin of replication which is inactive in the targeted receiver bacterial cell but is active in a donor bacterial cell, and said vector is devoid of antibiotic resistance marker,
thereby delivering said nucleic acid of interest into the targeted receiver bacterial cell, and
wherein, once delivered into said targeted receiver bacterial cell, said nucleic acid of interest produces said given effect on said targeted receiver bacterial cell while said vector is not replicated in said targeted receiver bacterial cell.

Unless defined otherwise, all technical and scientific terms used herein have the same meaning as commonly understood by one of ordinary skill in the art to which this invention belongs.

All publications mentioned herein are incorporated herein by reference. It is understood that the present disclosure supersedes any disclosure of an incorporated publication to the extent there is a contradiction.

It must be noted that as used herein, the singular forms “a”, “an” and “the” include plural referents unless the context clearly dictates otherwise. Thus, for example, reference to “a cell” includes a plurality of such cells (e.g., a population of such cells). Similarly, reference to “a nucleic acid” includes one or more of such nucleic acids.

The present invention will be further illustrated by the figures and examples below.

### BRIEF DESCRIPTION OF THE FIGURES

FIG. 1A-1B: BLAST analysis of the PICI-CFT073 origin in Escherichia coli.

FIG. 2A-2B: BLAST analysis of modified p15a origin of replication of sequence SEQ ID NO: 4 in Escherichia coli.

FIG. 3A-3B: BLAST analysis of the PICI-CFT073 origin in the domain Bacteria.

FIG. 4A-4B: BLAST analysis of the modified p15a origin in the domain Bacteria.

FIG. 5: Transformation of a 2.3 kb payload containing the primase-origin of replication (p1319) in a production strain harboring an inducible primase RBS library in trans.

FIG. 6: Comparison of packaged phagemids titers obtained with a plasmid containing the primase-ori in production strains (p1319) tested against 7 different primase RBS. Right column, in black, control plasmid with a p15a-derived origin of replication (p1220). Titers shown are after a 10× concentration.

FIG. 7: Comparison of cells transduced with a primase-ori plasmid (top row, p1319) and a p15a-based packaged phagemids (p1220) on LB agar plus 25 μg/mL chloramphenicol. RBS 3 is SEQ ID NO: 20.

FIG. 8: Comparison of killing activity in the absence of antibiotic selection of E. coli MG1655 transduced with a nuclease circuit targeting the lacZ gene. Black line, primase-ori (conditional replication, p1322); grey line, modified p15a-ori, replicative (p780).

FIG. 9: Packaged phagemids titers obtained with ˜12 kb plasmids harboring the mutated primase-ori in production strains containing primase RBS 3. Titers shown are after a 10× concentration. Left bar, lacZ target (p1326); right bar, 4stx target (p1327).

FIG. 10: Nuclease-mediated killing of different O157 strains mediated by targeting lacZ by transduction of packaged phagemids harboring a conditional origin of replication, payload p1326 (grey line c, an O157 strain lacking the lacZ gene serves as a non-killing control).

FIG. 11: Nuclease-mediated killing of four O157 strains mediated by stx targeting after transduction of packaged phagemids harboring a conditional origin of replication (payload p1327).

FIG. 12: Change in colonization in colonized mice orally administered with either a neutralizing buffer alone (‘+buffer’) or 10¹² particles of packaged phagemids in a neutralizing buffer (‘+EB’). Change in colonization between TO and T8h is reported for each animal, and the median and quartiles of each experimental group were graphed. ****p<0.0001 by unpaired t test.

FIG. 13: Adenine base editing of β-lactamase on the E. coli genome after phagemid transduction in vitro using a payload comprising a conditional origin of replication of sequence SEQ ID NO: 7. 96 individual colonies for each MOI were spotted on LB and LB (carbenicillin) plates and base editing efficiencies were calculated.

BRIEF DESCRIPTION OF THE SEQUENCES

| SEQ | | |
|---|---|---|
| ID NO: | Description | Type |
| 1 | insulin B9-25 epitope | Protein |
| 2 | T cell (β2GPI) epitope | Protein |
| 3 | B cell epitope | Protein |
| 4 | primase ori from the PICI of the E. coli strain CFT073 | DNA |
| 5 | Restriction site | DNA |
| 6 | Primase ori deltaGAAABCC | DNA |
| 7 | Primase ori devoid of restriction sites | DNA |
| 8 | PICI primase-helicase | Protein |
| 9 | PICI primase-helicase | DNA |
| 10 | payload p1392 plasmid | DNA |
| 11 | payload p1900 plasmid | DNA |
| 12 | chimeric STF (STF-V10-[Helix]) | Protein |
| 13 | chimeric STF (STF-V10-[Helix]) | DNA |
| 14 | chimeric gpJ (1A2) | Protein |
| 15 | chimeric gpJ (1A2) | DNA |
| 16 | p2.3 pri-ori p1319 | DNA |
| 17 | p2.8 p15a, p1220 | DNA |
| 18 | primase RBS 1 | DNA |
| 19 | primase RBS 2 | DNA |
| 20 | primase RBS 3 | DNA |
| 21 | primase RBS 4 | DNA |
| 22 | primase RBS 5 | DNA |
| 23 | primase RBS 6 | DNA |
| 24 | primase RBS 7 | DNA |
| 25 | plasmid lacz6 pri-ori, p1322 | DNA |
| 26 | primase RBS 11 | DNA |
| 27 | plasmid LacZ6 p15a, p780 | DNA |
| 28 | plasmid LacZ6 pri-ori deltaGAAABCC, p1326 | DNA |
| 29 | plasmid 4stx pri-ori deltagaaabcc, p1327 | DNA |
| 30 | β-lactamase gene | DNA |

### EXAMPLES

Packaged phagemids are being used to deliver a DNA payload to target bacteria with high efficiency. Features required for phagemid packaging are the presence of a packaging site and an origin of replication that is functional in the producer cell line.

The use of a constitutive origin of replication to produce packaged phagemids has several advantages, notably:
It can be stably maintained in production strains, simplifying the engineering, production and manufacturing processes,
Some constitutive ORIs compatible with lambda-based packaging, lead to sufficiently high titers (>10¹⁰/mL) required for microbiota-related applications (killing, delivery of therapeutic payloads, etc),
Since the payload will replicate in the target strain once injected, the effect of the expression of the gene of interest may be sustained long enough to have the desired outcome, for instance the killing efficiency may be higher when delivering a CRISPR-cas system targeted towards a chromosomal sequence, since it will be more difficult for the target strains to get rid of the payload by division: the residence time is increased.

Since phages have a precise tropism towards the same or closely related species in which they are produced, the packaged phagemids derived from this phage, once their payloads delivered in the target bacteria, will keep replicating, unless the phage has been engineered to infect/inject in a new group of bacteria.

However, having a phagemid harbouring a constitutive origin of replication may pose some risks when used in a clinical, industrial, or non-contained setup:
Since the payload is replicative, some events of injection will cause the plasmid to spread.
Moreover, when the payload is based on a common origin of replication present in many Enterobacteria (for example, a ColE-type origin), the risk of recombination with already-existing plasmids in target bacterial strains may be high. For regulatory purposes, this poses a problem since the transduced cells are considered as GMOs and are then replicative GMOs, which poses a containment risk that has to be evaluated accordingly.

For all these reasons, the inventors aimed to develop a conditional system of replication that encompasses all the advantages mentioned above while reducing the spread and recombination risks. Such a system needs to have the following features:
Replication of the payload must occur only in the production strain, the payload must be easy to maintain and be stable,
The system must allow for sufficiently high titers to be obtained (>10¹⁰/mL) to be relevant in an industrial setting,
The system must be amenable to sequence changes in case restriction sites need to be removed,
The system needs to be sufficiently rare in potential target strains as to reduce the risks of spread and recombination,
Finally, the system must allow for the gene of interest to be expressed and create the desired outcome (for instance killing of target strains at similar MOIs as when using replicative payloads).

In the following examples, the present inventors developed PICI-based conditional origins of replication.

First, they verified how common the origin region is in bacterial genomes, to assess the possibility of undesired recombination or payload spread events.

Second, they developed a system with the primase and ori in trans (ori on the phagemid
primase gene in the chromosome or on another plasmid carried by the bacteria) to assess if replication is truly conditional and dependant on the presence of the primase and to verify the titers obtained when such a system is used to package DNA payload.

Third, they tested in vitro killing of E. coli and compared it to the current generation of replicative payloads.

Finally, they assessed if the primase-origin was amenable to removal of undesired restriction sites.

In the following examples,
The inventors show for the first time that phagemids can be packaged at high titers with a conditional ORI,
The inventors show for the first time that phagemids can be packaged at high titers with a conditional ORI with ori and protein required for replication in trans,
The inventors show the additional advantage of using a ORI system that can be found in PICI genomes as opposed to other systems based on plasmid derived ORI (from a bacterial origin), which significantly limits the risk of spread. Furthermore, even if the ORI system is actually present in the transduced bacteria, meaning that a natural PICI harboring the same ORI system is found in the bacteria, it has to be active (in a lytic cycle) for the introduced phagemid to be replicated, since the primase gene in a PICI is inactive unless found in the induced (lytic) state. This is totally different for a bacterial ORI, since it would mean that it would be active naturally and constitutively.

### Example 1

### Blasting the Ori Region to Assess Frequency in E. coli and Other Bacteria

The 282 bp region right after the stop codon of the PICI-CFT073 primase (SEQ ID NO: 4) was used to BLAST against all sequenced Escherichia coli genomes, filtering to give up to 20,000 hits.

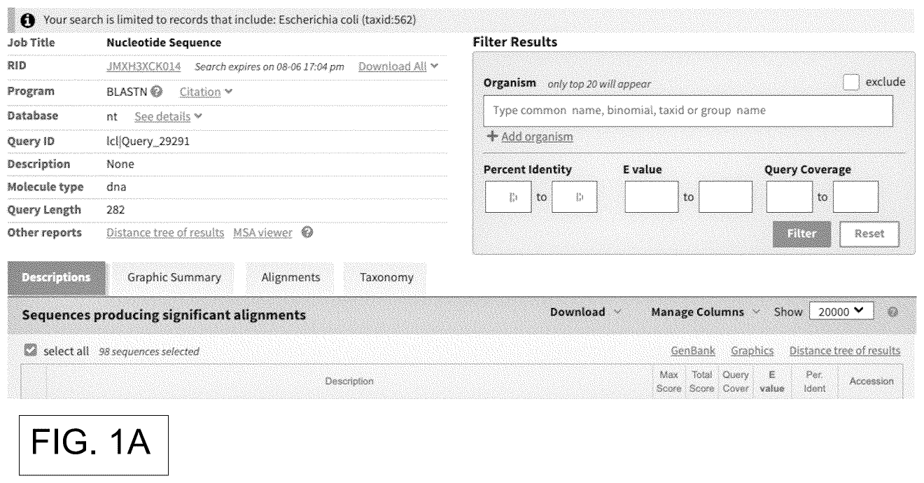

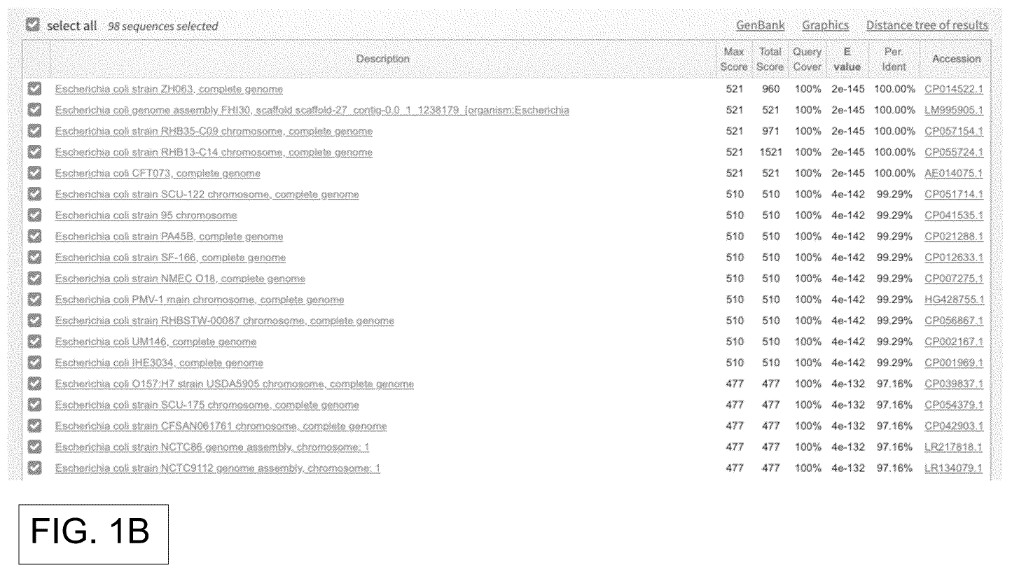

As shown in FIG. 1, out of all sequenced E. coli genomes, there are only 98 hits, which means that this specific primase-ori is very rare and hence, will drastically reduce the risk of recombination and replication in target and non-target strains.

It also needs to be noted that, under normal circumstances, the primase of the PICI is inactive, meaning that even if injection occurs in a strain containing this specific PICI, it will not replicate unless the cell is under a phage-induction state, which further reduces the chances of the introduced payload replicating when not desired.

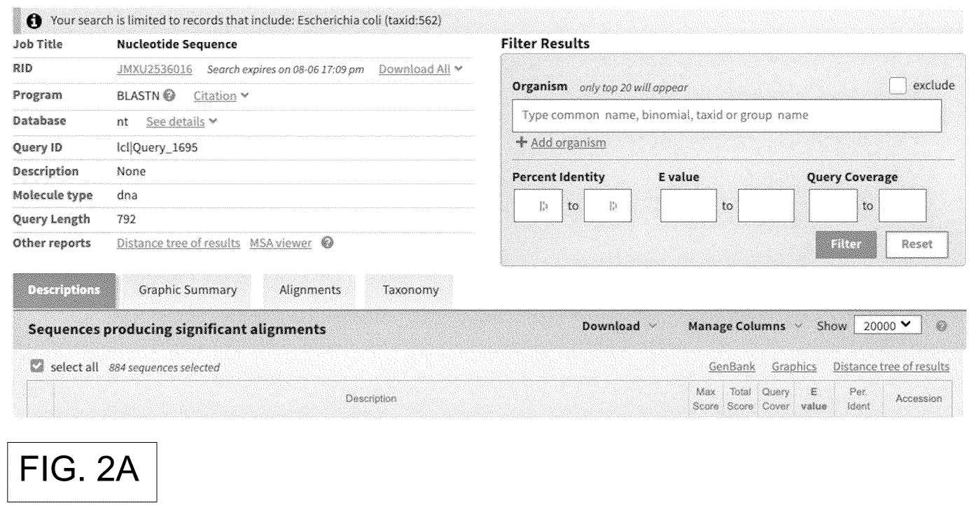

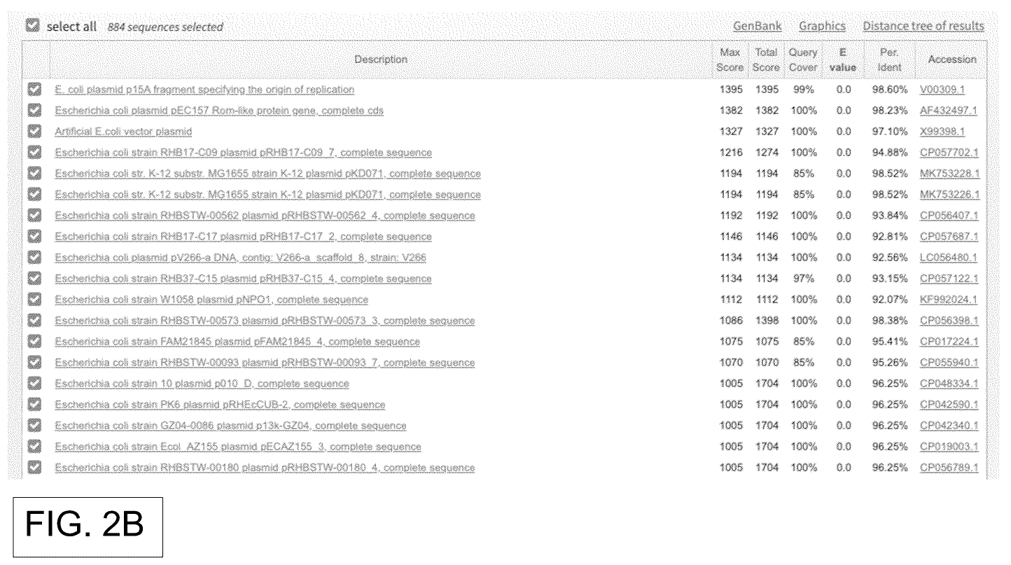

As a comparison, performing a BLAST analysis with a non-conditional modified p15a-based origin of replication returns the hits shown in FIG. 2.

884 sequences were found. It also needs to be noted that when sequencing strains, plasmids may be left out of the assembly if they are small (for example, the pOSAK found in STEC O157 strains), so the number of hits may be higher.

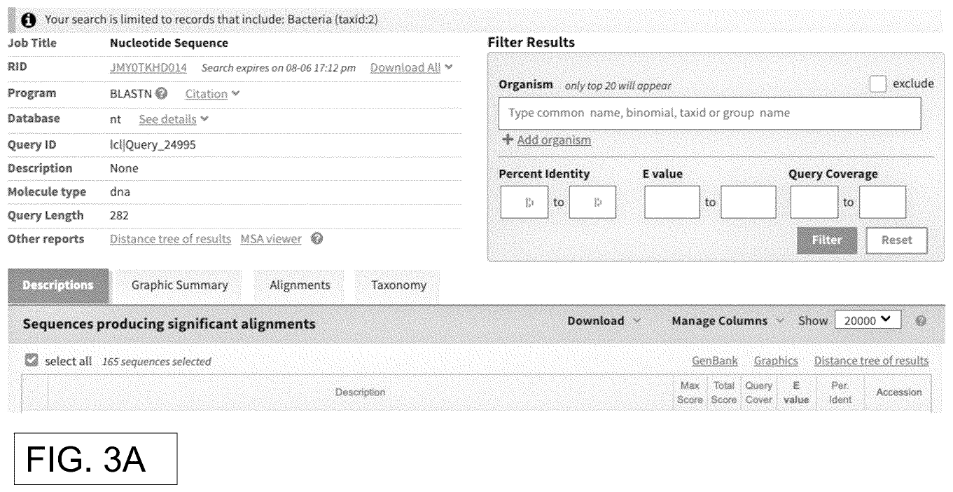

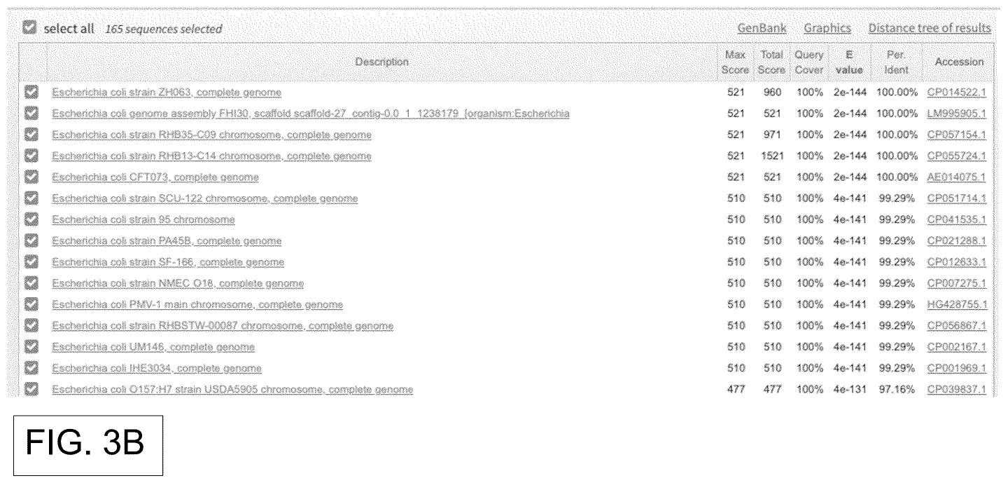

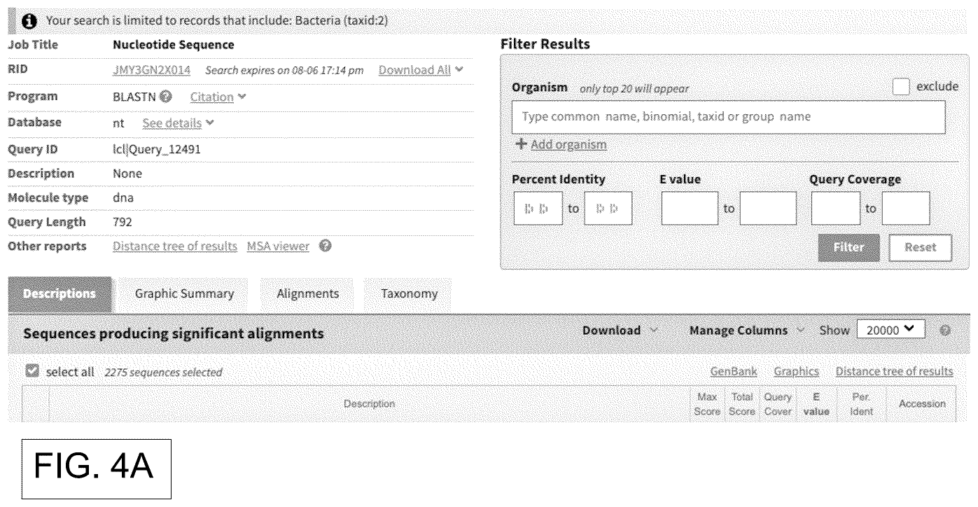

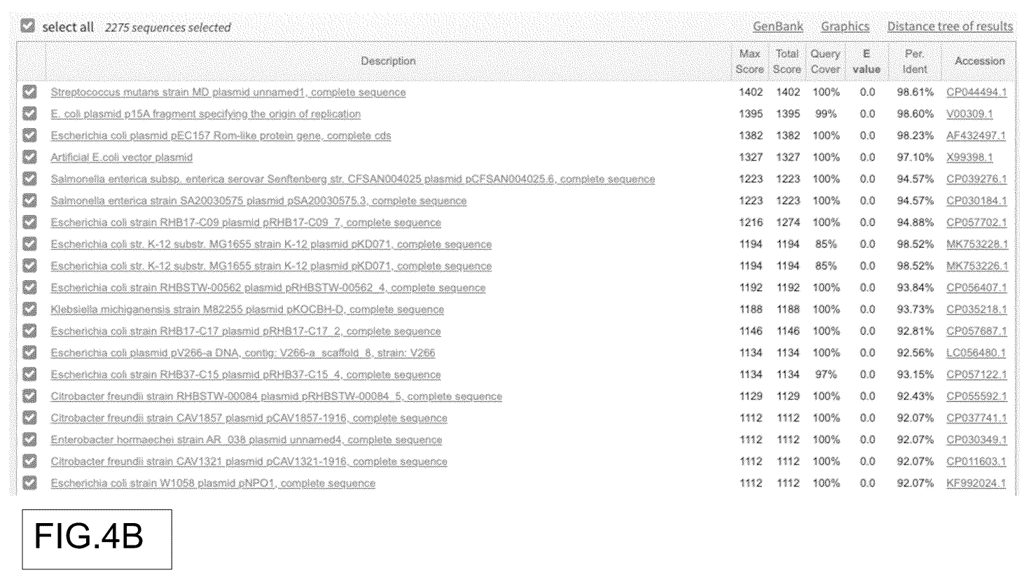

Next, the inventors performed the same search but this time using the Domain Bacteria to assess the presence of the PICI-ori in other non-E. coli species: 165 hits were found for the PICI origin while more than 2000 hits were found for the p15a-based origin (see FIGS. 3 and 4).

In conclusion the inventors showed that the primase-ori was a good candidate to reduce the risk of recombination and undesired replication in target and non-target bacteria since its occurrence, based on BLAST analyses, is 10 to 20 fold lower than a p15a-based origin; and for effective replication, the cell where the payload is injected will need to be undergoing active phage production for the PICI primase to be present.

### Example 2

### Developing a System with Primase-Ori in Trans Compatible with Phagemids Packaging

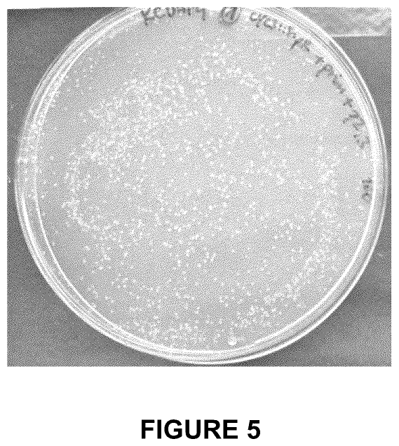

Next, the inventors sought to develop a system in which the payload contains the 282-bp primase origin and the primase protein is supplied in trans (SEQ ID NO: 8 and SEQ ID NO: 9). To simplify the engineering process, the PICI primase gene was extracted from the genome of E. coli CFT073, cloned into a plasmid under the control of an inducible system and an RBS (ribosome-binding site) library generated. This series of plasmids were cloned in the lambda production strain s1965. Next, the inventors constructed a small payload harboring the primase-ori instead of the p15a-based origin of replication to yield the 2.3 kb payload p1319 (SEQ ID NO: 16). Since this plasmid is, in principle, non-replicative, competent cells of s1965 harboring the RBS library of inducible primase constructs were made, the p1319 plasmid transformed in them and plated in LB agar+kanamycin and chloramphenicol in the presence of the inducer DAPG (to induce the expression of the primase in trans). Next day, the inventors observed that the plates contained hundreds of colonies, suggesting that the primase-origin system in trans works (FIG. 5).

Several clones were sequenced to verify that the p1319 plasmid contained no p15a-based origin and that they also contained an intact primase gene with an RBS coming from the library.

After that, 7 of these clones were grown overnight and lambda productions were carried out in the presence of kanamycin, chloramphenicol and DAPG. As a control, the inventors included the original 2.8 kb plasmid containing a derivative of the p15a origin of replication to compare titers (p1220, SEQ ID NO: 17)

To verify the sequence of the RBS variants obtained, the plasmid encoding the inducible primase in the 7 clones tested was miniprepped and sequenced (SEQ ID NO: 18 to 24). They were also transformed into MG1655 cells (s003): these strains were used to verify the titers obtained, since the payloads should not be replicative in the absence of the primase protein supplied in trans.

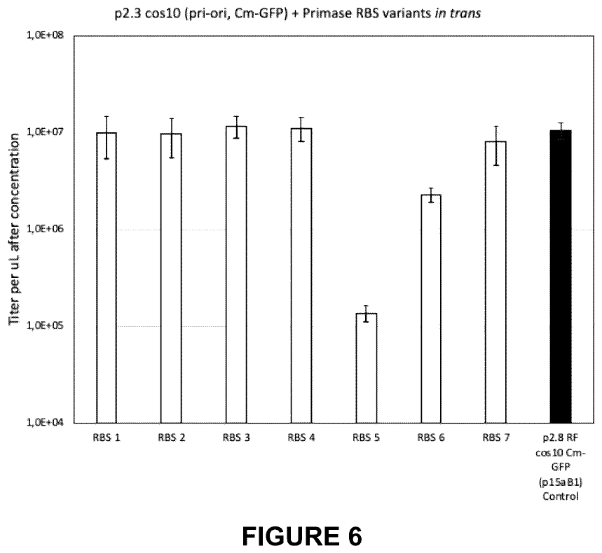

As can be seen on FIG. 6, the titers of 5 out of 7 primase-containing samples, as measured in MG1655 containing the primase plasmid in trans, were the same as those of a packaged phagemid carrying the original modified p15a origin.

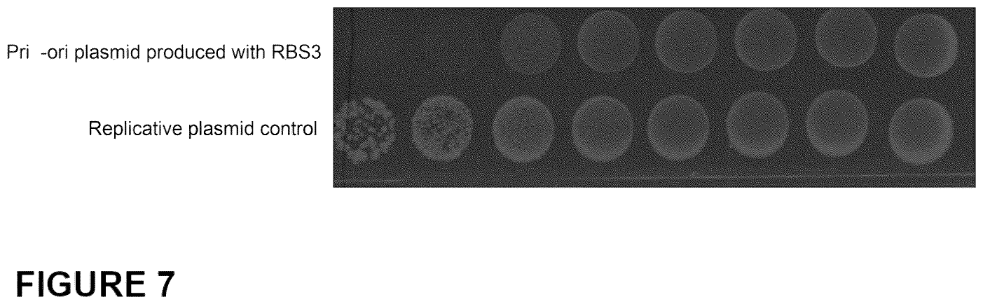

Finally, the inventors tested if the primase-ori containing payloads could replicate in MG1655 strains without the primase plasmid in trans. To do this, serial 5X dilutions of the primase-ori containing plasmids coming from the production strains with different primase RBS, plus a p15a-origin control, were transduced into a dense culture (OD600˜0.8) of MG1655 and plated on LB agar plates containing chloramphenicol. As can be seen on FIG. 7, while the p15a-origin control shows healthy colonies up to the last dilution, indicative of active plasmid replication, the samples containing the primase-containing payload show colonies only at high MOIs: since the strain will lose the payload by division, those drops that contained a high number of transduced bacteria will appear as dense spots since division will be halted at high cell densities; as the MOIs are reduced, the spots become more transparent and single colonies are hard to distinguish, indicative of cells that are dying due to plasmid loss and exposure to antibiotics. This is also indicative of a burst of expression of the chloramphenicol acetyltransferase gene upon transduction, which, in the absence of active replication, will get diluted over time; this may cause the receiver cells to survive for a certain amount of time until the intracellular concentration of chloramphenicol acetyltransferase drops below a critical level to support growth in antibiotic-supplemented media.

In conclusion, PICI primase and origin can be stably maintained in production strains, are compatible with lambda-based phagemids packaging judging by the titers obtained and the payloads are dependent on the presence of its cognate primase for active replication and maintenance in target strains.

### Example 3

### In Vitro Killing of E. coli Using a Conditional Origin of Replication

Next, the inventors tested if sequence-specific killing mediated by the Cpf1 nuclease would still occur in cells transduced by packaged phagemids. Since the cells will lose the plasmid by division, it was ignored if the initial burst of expression of the nuclease circuit would still be sufficient to achieve killing at a similar MOI as the one observed with a constitutive origin of replication.

To do this, the inventors constructed a large plasmid (˜12 kb) exchanging the p15a-based origin of replication by the primase origin. This plasmid targets the lacZ gene (p1322, SEQ ID NO: 25) and also contains a chloramphenicol marker. Since it was ignored if the RBS strength would need to be modified to replicate a large plasmid, the inventors transformed this plasmid into the production strain s1965 harboring an inducible primase RBS library in trans, as done for the initial, smaller payload. Next day, the inventors observed that the plates contained hundreds of colonies. One of these colonies was picked, sequenced to verify that the payload contained the primase-ori, the RBS of the primase in trans sequenced (SEQ ID NO: 26) and packaged phagemids were produced. As a control, the inventors produced the same phagemid containing a p15a-based origin of replication (p780, SEQ ID NO: 27) from the same production strain.

In this case, since the payload targets the MG1655 strain, the inventors verified the titers of the production in a derivative of MG1655 lacking the lacZ gene (s248) and containing the primase RBS 3 plasmid in trans (p1321).

Titers of both packaged phagemids whose payloads comprise constitutive and conditional origins of replication were undistinguishable, of about 1.5×10⁸/μL after 10× concentration, suggesting that this approach is also valid for larger payloads.

Next, the inventors tested if killing of a target strain with packaged phagemids would be possible in the absence of selection and active replication of the payload, as the inventors already demonstrated with p15a-based origins. To do this, a culture of E. coli MG1655 was grown in LB+CaCl₂ to an OD600 of about 0.8 and diluted in LB+CaCl₂ to an OD=0.025. The packaged phagemids targeting lacZ and containing the p15a-based origin (control) or the primase origin were serially diluted 3×; this approach allowed for testing different MOIs. 90 μL of cells were added to each well containing a packaged phagemid dilution. After 30 min-incubation at 37° C., 10× dilutions of each reaction were performed, 10 μL plated on LB agar plates and incubated overnight at 37° C.

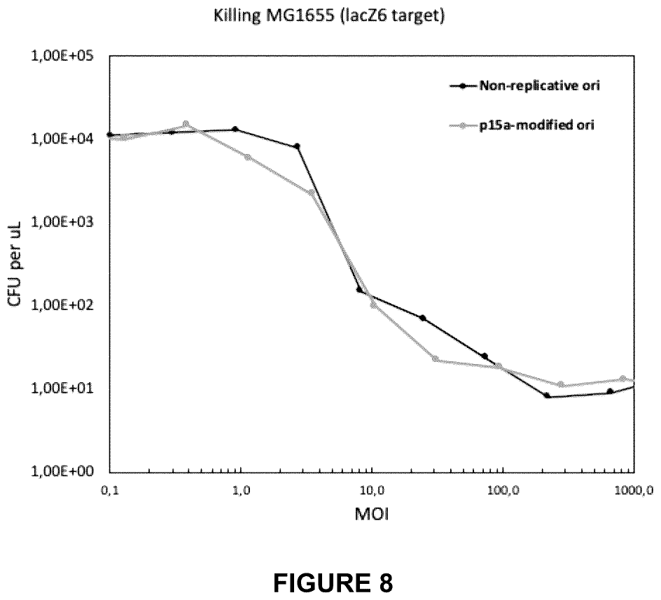

As can be seen in FIG. 8, the behavior of the p15a-containing nuclease payload was indistinguishable from the payload containing the primase conditional origin: about 2-log killing at an MOI 10.

In conclusion, conditional origins of replication based on PICIs allow for production at high titers of large payloads (˜12 kb) and nuclease-mediated killing of a target strain in the absence of selection and primase protein.

### Example 4

### Removal of Restriction Sites from Pici-Derived Origins of Replication

Finally, the inventors tested if the PICI origins of replication were amenable to removal of restriction sites present in certain target strains: the presence of such sites may completely abolish nuclease-specific killing since the payload will be degraded in the target strain before the nuclease gene is expressed.

To do this, the inventors analyzed the 282-bp PICI origin and found that it contains the O157 restriction site GAAABCC (GAAAGCC). The inventors modified this site within the origin and obtained the sequence GAAAGCa (small cap represents the mutation introduced) which should not be recognized by O157 strains. The modified PICI origin (SEQ ID NO: 6) was then cloned into ˜12 kb payloads containing a Cpf1 nuclease circuit targeting the lacZ gene as mentioned in Example 3 (p1326, SEQ ID NO: 28) and also a quadruplex crRNA guide targeting stx1 and stx2 genes (p1327, SEQ ID NO: 29).

The inventors previously designed a bacterial cell line producing an engineered lambda-based capsid, comprising a chimeric 1A2 gpJ protein and a chimeric STF-V10[Helix], able to inject efficiently in O157 strains (s15816), so these two plasmids were transformed in this production strain containing the primase RBS 3 in trans.

Colonies were readily obtained, which suggested that the mutation introduced in the origin does not affect the ability of the PICI primase to recognize and replicate it. Sequencing results verified the presence of a modified, deltaGAAABCC primase origin of replication.

Packaged phagemids were produced from these two strains and titrated on a variant of MG1655 recognized by this specific packaged phagemid, supplemented with a plasmid encoding the primase RBS variant 3 (s18241).

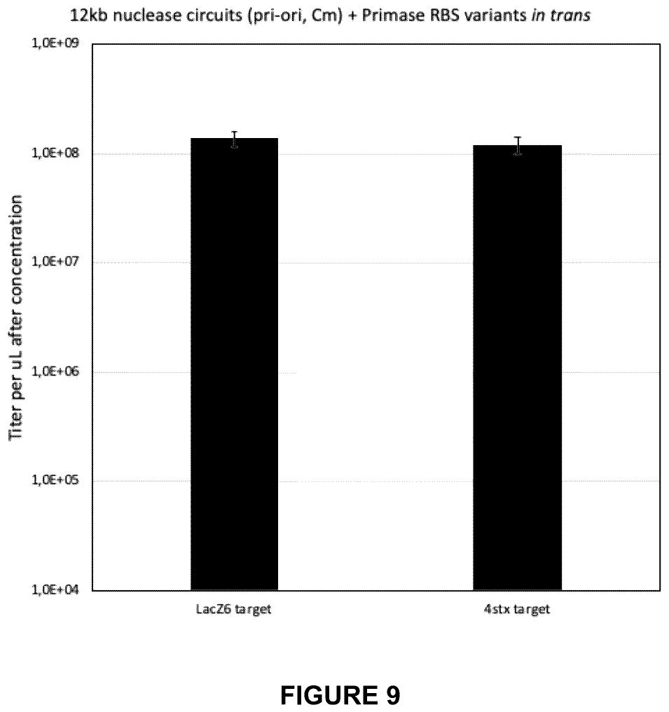

As can be seen on FIG. 9, titers are equivalent with both p15a-containing origins or non-mutated PICI origins (>1×10⁸/μL after 10× concentration).

Finally, two killing experiments were performed in O157 strains as described above for MG1655:
Killing using the lacZ target in two O157-delta-stx strains (s2185 and s17465). As a control for unspecific killing, packaged phagemids were also transduced into the strain s11983, which is a derivative of the O157 H10dstx strain lacking the lacZ gene.
Killing using the quadruplex crRNA guides targeting stx targets in four wild-type O157 strains (s13861, s13862, s13863, s13864).

Briefly, cell cultures were brought to an OD600=0.025 and packaged phagemids serially diluted 1:3. 90 μL of cell cultures were added to the packaged phagemid dilutions, incubated for 30 min at 37° C., and serial 10× dilutions to allow for cell count were performed. 10 μL of each dilution were then plated on LB agar.

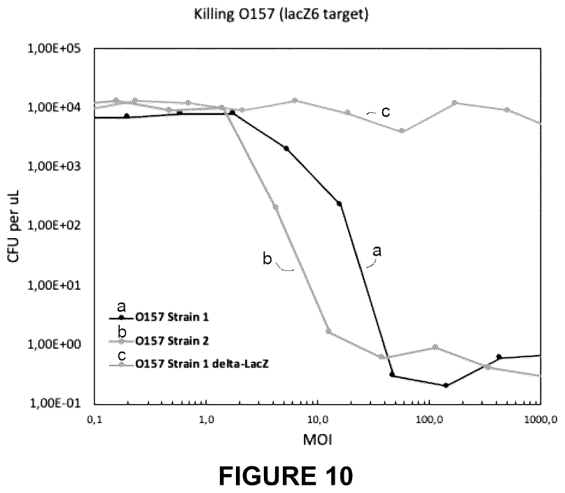

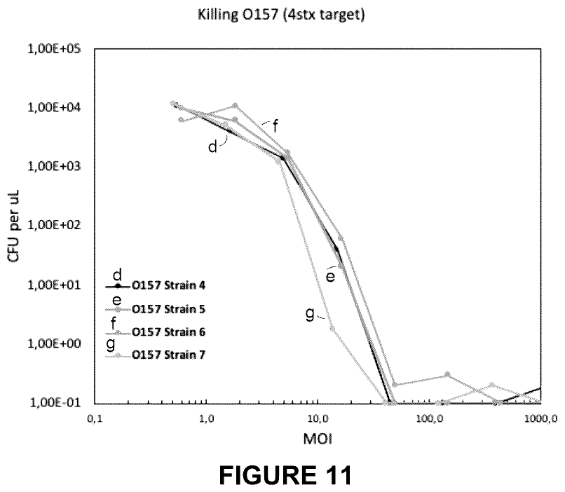

As can be seen on FIGS. 10 and 11, both packaged phagemids targeting lacZ or stx genes are effective and the MOIs needed for killing are equivalent to those obtained with packaged phagemids containing constitutive origins of replication in the absence of antibiotic selection. Strains not containing the target (s11983) are not killed at all, as expected, which suggests there is little to no nonspecific-killing. Additionally, when plated on selection media (LB agar containing chloramphenicol), the non-targeted strain shows a similar profile as that seen for MG1655: dense spots at high MOIs and low dilutions (the cells cannot actively divide due to cell density and cannot lose the plasmid) and weaker density spots, translucid, at lower MOIs and higher dilutions, indicative of cell death due to exposure to the antibiotics.

### Example 5

### In Vivo Decolonization with a Payload Bearing a Conditional Origin of Replication

The present example demonstrates efficient decolonization in vivo by specifically killing bacteria bearing six genes using a packaged phagemid with a conditional origin of replication.

### Materials and Methods

Streptomycin-treated mice were orally administered with either a target bacterial strain (hereafter referred to as ‘Target strain’) or a mutant of the same bacterial strain deleted for a specific gene of interest, namely a stx gene (hereafter referred to as ‘Non-Target strain’) to establish a durable intestinal colonization with these bacterial strains.

A plasmid of sequence SEQ ID NO: 10, carrying a conditional origin of replication of sequence SEQ ID NO: 7, and coding for a nuclease and its guide targeting the stx gene mentioned above, was packaged into an engineered lambda-based capsid, comprising a chimeric 1A2 gpJ protein and a chimeric STF-V10[Helix] (1A2-V10 packaged phagemid).

Mice colonized with either strain were given 100 μl of packaged phagemids (approximately 10¹² particles) along with 100 μl of a buffer (sucrose and bicarbonate in water) aimed at temporarily neutralizing the gastric pH. A separate group of mice colonized with the Target strain received only the buffer, to account for natural changes in colonization levels over the time of the experiment.

The bacterial colonization levels were measured non-invasively by plating dilutions of stool recovered from each animal individually onto agar plates.

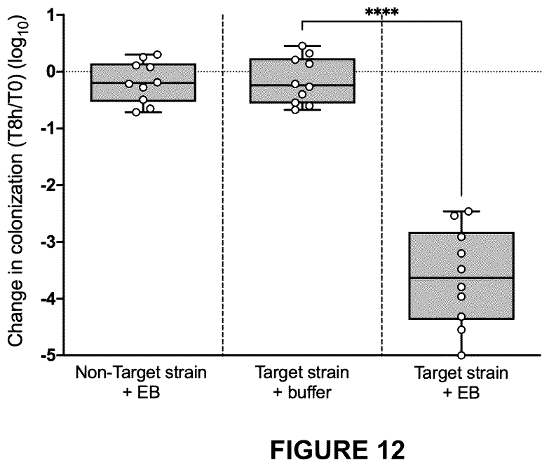

These levels were compared before treatment was initiated (termed ‘T0’) and 8 hours after the treatment (termed ‘T8h’), and the change in colonization between T8h and T0 was calculated for each animal, and expressed as logarithmic change (see FIG. 12).

### Results

The pH-neutralizing buffer alone had no effect on the Target strain colonization levels, whereas the packaged phagemids caused a 3.5-log reduction in bacterial burden recovered from the stool 8 hours after oral administration. As expected, the packaged phagemids had no effect on colonization levels of the Non-target strain, demonstrating the specificity of packaged phagemids towards their target sequence.

These results thus demonstrate that an efficient in vivo killing of targeted bacteria can be achieved by delivering in said targeted bacteria, packaged phagemids with a conditional origin of replication, which is not active in the targeted bacteria, said phagemids being this incapable to replicate in said targeted bacteria.

### Example 6

### Adenine Base Editing of β-Lactamase on the E. coli Genome after Phagemid Transduction In Vitro Using a Payload with a Conditional Origin of Replication

This example presents a method for the base editing of the nucleic acid sequence encoding ®-lactamase (SEQ ID NO: 30) on the E. coli MG1655 genome after phagemid transduction in vitro using a payload comprising a conditional origin of replication of sequence SEQ ID NO: 7, based on a primase-helicase.

The non-replicative payload comprises an adenine base editor (ABE8e), a transcribed guideRNA targeting the active site of the p-lactamase gene (K71E) on the genome, a lambda packaging sequence, a chloramphenicol resistance marker, and the conditional origin of replication of sequence SEQ ID NO: 7. Production of lambda phagemids, packaged inside a bacterial delivery vehicle comprising an A8 gpJ protein and an EB6 STF protein for delivery into E. coli MG1655, resulted in titers of 6.7×10⁶ transduction units per μl (tu/μl).

Transduced cells were plated on LB plates 2 hours post transduction at different multiplicity of infections (MOI). The next day, 96 individual colonies for each MOI were spotted on LB and LB (carbenicillin) plates in order to analyse the base editing efficiency.

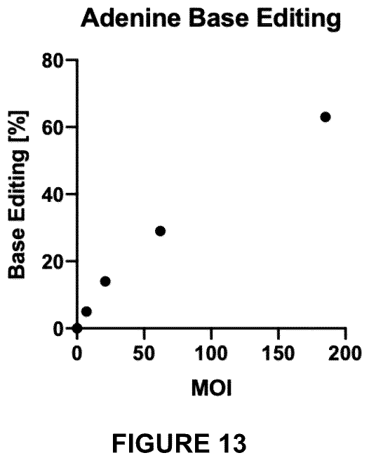

As shown on FIG. 13, the efficiency of adenine base editing targeting the active site of the β-lactamase gene (K71E) on the genome was multiplicity of infection (MOI)-dependent. A base editing efficiency of ˜63% of the bacterial population was obtained at high MOIs using the payload comprising a conditional origin of replication.

1. A nucleic acid vector encoding a programmable nuclease, wherein said programmable nuclease kills a targeted receiver bacterial cell,
wherein said vector is a phagemid or recombinant phage nucleic acid vector, said vector comprising a conditional origin of replication which is inactive in the targeted receiver bacterial cell but is active in a donor bacterial cell, wherein said conditional origin of replication is SEQ ID NO: 4 which is the origin of replication from the phage-inducible chromosomal island (PICI) of the Escherichia coli strain CFT073, or said origin of replication is modified to be the sequence of SEQ ID NO: 6 or SEQ ID NO: 7, and wherein said conditional origin of replication is active in said donor bacterial cell because said donor bacterial cell expresses a primase-helicase comprising SEQ ID NO: 8 and wherein said vector is devoid of antibiotic resistance marker.

2. The nucleic acid vector of claim 1, wherein the nuclease is selected from the group consisting of Cpf1 nuclease, Cas9 nuclease, Mad4 nuclease, Mad7 nuclease, and Cms1 nuclease.

3. The nucleic acid vector of claim 1, wherein the nuclease is Cpf1 nuclease.

4. The nucleic acid vector of claim 1, wherein the nuclease is Cas9 nuclease.

5. The nucleic acid vector of claim 1, wherein the nuclease is Mad4 nuclease.

6. The nucleic acid vector of claim 1, wherein the nuclease is Mad7 nuclease.

7. The nucleic acid vector of claim 1, wherein the nuclease is Cms1 nuclease.

8. A bacterial delivery vehicle comprising the vector according to claim 1.
